(19) United States
(12) Patent Application Publication
(10) Pub. No.: US 2014/0086911 A1
(43) Pub. Date: Mar. 27, 2014

(54) Methods and Products for Predicting CMTC Class and Prognosis in Breast Cancer Patients

(71) Applicant: University Health Network, Toronto (CA)

(72) Inventors: Wey Liang Leong, Toronto (CA); Dong-Yu Wang, Toronto (CA); David R. McCready, Toronto (CA); Susan Jane Done, Toronto (CA)

(73) Assignee: University Health Network, Toronto (CA)

(21) Appl. No.: 14/032,831

(22) Filed: Sep. 20, 2013

Related U.S. Application Data
(60) Provisional application No. 61/704,130, filed on Sep. 21, 2012.

(51) Int. Cl. C12Q 1/68

(52) U.S. Cl. CPC C12Q 1/6886
USPC 424/133.1; 435/6.12; 506/9; 514/651

(57) ABSTRACT

Provided herein are products, uses and method classifying a subject afflicted with breast cancer according to a ClinicoMolecular Triad Classification (CMTC)-1, CMTC-2 or CMTC-3 class. The method involves:
(i) determining a subject expression profile, said subject expression profile comprising the mRNA expression levels of a plurality of genes that classify breast cancer into three groups by hierarchal clustering TN and Her2+ breast cancers into one class (CMTC genes), in a breast cancer cell sample taken from said subject;
(ii) calculating a measure of similarity between said subject expression profile, and one or more of: a) a CMTC-1 reference profile, b) a CMTC-2 reference profile, and c) a CMTC-3 reference profile; and
(iii) classifying said subject.

### CROSS REFERENCE TO RELATED APPLICATIONS

This application claims the benefit of 35 USC 119 based on the priority of U.S. Provisional Application No. 61/704,130 filed Sep. 21, 2012, which is herein incorporated by reference.

### FIELD

The disclosure relates to methods and products for classifying a subject afflicted with breast cancer according to three clinical treatment classes that are associated with prognosis.

### BACKGROUND

The presence of estrogen receptor (ER), progesterone receptor (PR) and human epidermal growth factor receptor 2 (Her2, also known as ERBB2) is routinely reported in the pathological assessment of breast cancer. These three receptors have become the mainstay of clinical and molecular classification of breast cancer [1,2]. In general, positive ER and PR status (ER+ and PR+, respectively) are considered good prognostic indicators, whereas positive Her2 status is considered a poor prognostic indicator [2]. However, negative status in all three receptors, that is, ER, PR− and Her2−, also referred as “triple-negative” (TN) status, is also considered a poor prognostic indicator [3]. Because most basal-like subtype tumors are TN, these terms have been used interchangeably, but in actual fact TN and basal-like breast cancer are not the same and some of them can be differentiated from each other by more in-depth molecular characterization [3-5]. Oncologists generally divide breast cancer into three clinically relevant groups when making treatment decisions. Group 1 breast cancers are generally low-risk and ER+ and respond well to endocrine therapy (ET), such as tamoxifen. Group 2 breast cancers are ER+ but carry a poor prognosis despite ET, and therefore chemotherapy is strongly recommended for patients in this group. Group 3 breast cancers are ER−, including Her2+ and TN cancers with a poor prognosis that generally improves with chemotherapy, as well as trastuzumab if necessary.

There is a need to find a new personalized test for breast cancer (BC) because current use of population-based prognostic systems to make treatment decisions is inaccurate and associated with over-prescription of systemic therapies. Two multigene tests, Oncotype DX™ (21-gene) [45] and MammaPrint™ (70-gene) [56] exist, but have limitations including restricted patient eligibilities (e.g. only estrogen receptor-positive tumours for Oncotype DX, fresh frozen tissue for MammaPrint).

### SUMMARY

An aspect of the disclosure includes a method for classifying a subject afflicted with breast cancer according to a ClinicoMolecular Triad Classification (CMTC)-1, CMTC-2 or CMTC-3 class, the method comprising:
(i) determining a subject expression profile, said subject expression profile comprising the mRNA expression levels of a plurality of genes that classifies breast cancer into three groups by which the two worst molecular subtypes (i.e. TN and Her2+) are grouped into one class, in a breast cancer cell sample taken from said subject;
(ii) calculating a measure of similarity between said subject expression profile, and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients; and
(iii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

In an embodiment, the plurality of genes are selected from Table 9.

In another embodiment, the method comprises:
(i) determining a subject expression profile said subject expression profile comprising the mRNA expression levels of a plurality of genes, the plurality comprising at least 200, at least 300, at least 400, at least 500, at least 600, at least 700 or at least 800 genes, optionally at least 200, at least 300, at least 400, at least 500, at least 600, at least 700, at least 800 or 803 of the genes listed in Table 9 in a breast cancer cell sample taken from said subject;
(ii) calculating a measure of similarity between said subject expression profile, and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients; and
(iii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

In another embodiment, said similarity is assessed by calculating a correlation coefficient between the subject expression profiles and the one or more of CMTC-1, CMTC-2 and CMTC-reference profiles, wherein the subject is classified as falling in the class that has the highest correlation coefficient with the subject expression profile.

In certain embodiments, step (iii) alternatively or in addition comprises classifying said subject as having a poor prognosis if said subject expression profile has a high similarity to or is most similar to said CMTC-3 reference profile or said CMTC-2 reference profile, classifying said subject as having a good prognosis if said subject expression profile has a high similarity to or is most similar to said CMTC-1 reference profile; and providing said prognosis classification to the subject.

In an embodiment, the method further comprising (iii) displaying; or outputting to a user interface device, a computer-readable storage medium, or a local or remote computer system, the classification produced by said classifying step (ii).

In an embodiment, said CMTC reference profile comprises for at least 200, at least 300, at least 400, at least 500, at least 600, at least 700, or at least 800, genes optionally at least 200, at least 300, at least 400, at least 500, at least 600, at least 700, at least 800 or 803 genes in Table 9 or at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes in Table 9, respective centroid values optionally for example for Table 9 genes, respective centroid values listed in Table 9.

In certain embodiments, the method comprising obtaining a breast cancer cell sample and/or assaying the sample and determining a subject expression profile.

In an embodiment, the method comprises;
a. obtaining a breast cancer cell sample from the subject;
b. assaying the sample and determining a subject expression profile, said subject expression profile comprising the mRNA expression levels of a plurality of genes, the plurality comprising at least 200, at least 300, at least 400, at least 500, at least 600, at least 700, or at least 800 genes, optionally at least 200, at least 300, at least 400, at least 500, at least 600, at least 700, at least 800, or 803 of the genes listed in Table 9 in a breast cancer cell sample taken from said subject
c. comparing the subject expression profile to one or more of a CMTC-1, CMTC-2 and/or CMTC-3 reference profile, said CMTC-1 reference profile comprising expression levels of said plurality of genes that are average mRNA expression levels of said respective genes in breast cancer cells of a plurality of ER+ low proliferating breast cancer patients, said CMTC-2 reference profile comprising expression levels of said plurality of genes that are average mRNA expression levels of said respective genes in breast cancer cells of a plurality of breast cancer patients having ER+ high proliferating breast cancer; and said CMTC-3 reference profile comprising expression levels of said plurality of genes that are average mRNA expression levels of said respective genes in breast cancer cells of a plurality of triple negative and HER2+ breast cancer patients;
d. classifying said subject as falling within a CMTC-1 class if said subject expression profile has a higher similarity to the CMTC-1 reference profile than the CMTC-2 or CMTC-3 reference profiles; classifying said subject as falling within a CMTC-2 class if said subject profile has a higher similarity to the CMTC-2 reference profile than the CMTC-1 or CMTC-3 reference profiles; and classifying said subject as falling within a CMTC-3 class if said subject profile has a higher similarity to the CMTC-3 reference profile than the CMTC-1 or CMTC-2 reference profiles.

In certain embodiments, said plurality of genes comprises at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and optionally at least 97%, at least 98%, at least 99% or 100% of the genes listed in Table 9.

In certain embodiments, said expression level of each gene in said subject expression profile is a relative expression level of said gene in said breast cancer cell sample versus expression level of said gene in a reference pool, optionally represented as a log ratio and/or, wherein said reference profile comprising expression levels of the plurality of genes is an error-weighted average.

The disclosure in another aspect includes a method for monitoring a response to a cancer treatment in a subject afflicted with breast cancer, comprising:
a. collecting a first breast cancer cell sample from the subject before the subject has received the cancer treatment or during treatment and collecting a subsequent breast cancer cell sample from the subject after the subject has received at least one cancer treatment dose;
b. assaying said first sample and determining a first subject expression profile, said first subject expression profile comprising the mRNA expression levels of a plurality of genes of said first breast cancer cell sample and assaying and determining a second subject expression profile, said second subject expression profile comprising the mRNA expression levels of said plurality of genes of said subsequent breast cancer cell sample, said plurality of genes optionally comprising at least 200, at least 300, at least 400, at least 500, at least 600, at least 700, or at least 800, genes, optionally comprising at least 200 genes at least 200, at least 300, at least 400, at least 500, at least 600, at least 700, at least 800 or 803 genes listed in Table 9;
c. classifying said subject as having a good prognosis, or a poor prognosis or CMTC class based on said first subject expression profile and classifying said subject as having a good prognosis or a poor prognosis or CMTC class based on said second subject expression profile according to a method described herein;
d. and/or calculating a first sample subject expression profile score and a subsequent sample subject expression profile score;
wherein a lower subsequent sample expression profile score or better prognosis class compared to the first sample expression profile score is indicative of a positive response, and a higher subsequent sample expression profile score or worse class compared to said first sample subject expression profile score is indicative of a negative response.

In certain embodiments, each of said mRNA expression levels is determined using one or more probes and/or one or more probe sets, optionally wherein the one or more polynucleotide probes and/or the one or more polynucleotide probe sets are selected from the probes identified by number in Table 9.

In another embodiment, the mRNA expression level is determined using an array and/or PCR method, optionally multiplex PCR, optionally, wherein the array is selected from an Illumina™ Human Ref-8 expression microarray, an Agilent™ Hu25K microarray and an Affymetrix™ U133 or other microarray comprising probes for detecting gene expression for example of at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes in Table 9.

In an embodiment, the method comprises: (a) contacting first nucleic acids derived from mRNA of a breast cancer cell sample taken from said subject, and optionally a second nucleic acids derived from mRNA of two or more breast cancer cell samples from breast cancer patients who have recurrence within a predetermined period from initial diagnosis of breast cancer and/or known ER/PR/HER2 clinical status, with an array under conditions such that hybridization can occur, wherein the first nucleic acids are labeled with a first fluorescent label, and the optional second nucleic acids are labeled with e second fluorescent label, detecting at each of a plurality of discrete loci on said array a first fluorescent emission signal from said first nucleic acids and optionally a second fluorescent emission signal from said second nucleic acids that are bound to said array under said conditions, wherein said array optionally comprises at least 200 optionally at least 200 of the genes listed in Table 9; (b) calculating a first measure of similarity between said first fluorescent emission signals and said second fluorescent emission signals across said at least 200 genes or calculating one or more measures of similarity between said first fluorescent emission signals and one or more reference profiles; (c) classifying said subject based on the similarity between said first fluorescent emission signals and said second fluorescent emission signals across said at least 200 genes or based on the similarity between said first fluorescent emission signals and said one or more reference profiles across said at least 200 genes (e.g. CMTC-1, CMTC-2, CMTC-3 or good, or poor prognosis reference profiles) wherein said individual is classified as having a good prognosis if said subject expression profile is most similar to a CMTC-1 reference profile or a poor prognosis if said subject expression profile is most similar to said CMTC-2 or CMTC-3 reference profile; and (d) displaying; or outputting to a user interface device, a computer readable storage medium, or a local or remote computer system; the classification produced by said classifying step (c).

Also provided in another aspect is a method of treating a subject afflicted with breast cancer, comprising classifying said subject according to a method described herein, and providing a suitable cancer treatment to the subject in need thereof according to the class determined.

A further aspect includes a method for classifying a remotely obtained breast cancer sample according to CMTC and providing access to the CMTC classification of the breast cancer cell sample, the method comprising:
receiving a remotely obtained breast cancer cell sample and a breast cancer cell sample identifier associated to the breast cancer cell sample;
determining on-site the expression levels for a plurality of genes of the received cell sample;
classifying the breast cancer cell sample according to CMTC;
providing access to the CMTC classification for the breast cancer cell sample.

Yet a further aspect includes kit for determining CMTC class in a subject afflicted with breast cancer according to the method described herein comprising one or more of:
a needle or other breast cancer cell sample obtainer;
tissue RNA preservative solution;
breast cancer cell sample identifier;
vial such as a cryovial; and
instructions.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 shows CMTC gene expression pattern, prognostic framework, and oncogenic pathway activity. (A) The 803-gene signature (represented by 828 oligonucleotide probes) was used to classify the gene expression pattern in the 149 breast cancers in the training cohort into the three main clusters of CMTC. Tumors in CMTC-3 were mostly Her2+/TN as well as CMTC-1 and CMTC-2 non-Her2+/TN. The bottom multicolor bars indicating Her2+/TN are as follows: Her2+, lighter; TN, darker. The bars indicating grade are as follows: grade 1, white; grade 2, grey; and grade 3, black. CMTC=ClinicoMolecular Triad Classification; Her2=human epidermal growth factor receptor 2; TN=triple-negative; ER2+=estrogen receptor-positive; TGF.beta.RII=transforming growth factor beta receptor type II. 1: Her2+/TN; 2: ER+; 3: grade; 4: recurrence; 5: 37GS poor; 6: 70GS poor; 7: 76GS poor; 8: 97GS poor; 9: ERGS poor; 10: ESGS poor; 11: IGS poor; 12: P53GS poor; 13: PAM50; 14: proliferation high; 15: SDPP poor; 16: subtype; 17: TGF.beta.RII deficient; 18: WS poor.

(B) The probabilities of pathway activation of 19 published oncogenic pathway signatures in the 149 breast cancers in the training cohort. Darker shading indicates low pathway activity, and lighter shading indicates high activity. EGFR=epidermal growth factor receptor; PAM50=50-gene prediction analysis of microarray; PI3K=phosphatidylinositol 3-kinase; PR=progesterone receptor; STAT3=signal transducer and activator of transcription 3. 1: E2F3; 2: Src; 3:EGFR; 4: P13k; 5: p53; 6: ER; 7: PR; 8: TGF.beta; 9: Akt; 10: p63; 11: MYC; 12: E2F1; 13: beta-catenin; 14: Ras; 15: Her2; 16: STAT3; 17: TNF.alpha; 18: IFN.gamma; 19: IFN.alpha.

FIG. 2 illustrates the CMTC and Her2+/TN status in prediction of the clinical outcomes. Kaplan-Meier analysis was used to compare relapse-free patient survival among the CMTC-1 (C1), CMTC-2 (C2) and CMTC-3 (C3) in (A) 2,239 breast cancers overall and (B) 1,058 nonadjuvant treatment cancers, as well as in (C) Her2+/TN and non-Her2+/TN 2,239 breast cancers overall and (D) 1,058 nonadjuvant treatment cancers. The hazard ratios with 95% confidence intervals in parentheses were calculated using the Cox proportional hazards method. The P values were determined using the log-rank test. CMTC=ClinicoMolecular Triad Classification; Her2=human epidermal growth factor receptor 2; HR=hazard ratio; TN=triple-negative.

FIG. 3 shows CMTC and the prediction of benefits of ET in ER+ breast cancer. Kaplan-Meier analysis was used to compare patients' relapse-free survival (A) between ET treatment and no treatment (B) among all 756 ER+ breast cancers, (C) between the three CMTC groups of all 756 ER+ breast cancers and ET treatment vs no treatment in 299 CMTC 1-only ER+ cancers and (D) and ET treatment vs no treatment between 457 CMTC-2- and CMTC-3-only ER+ cancers. The P values were determined using the log-rank test. CMTC=ClinicoMolecular Triad Classification; ER=estrogen receptor; ET=endocrine therapy; TN=triple-negative.

FIG. 4 shows CMTC and prediction in pCR of neoadjuvant chemotherapy. (A) The percentage of pCR between non-Her2+/TN tumors (non-H+/TN) and Her2+/TN tumors (H+/TN) and within the three CMTC groups of the 248 breast cancers with neoadjuvant chemotherapy. (B) Comparison of area under the curve (AUC) to predict pCR in CMTC-3 tumors (CMTC-3 vs CMTC-1 and CMTC-2; P=0.0001), Her2+/TN tumors (Her2+/TN vs non-Her2+/TN; P=0.0001), Her2+ tumors (Her2+vs Her2−; P=0.0245) and TN tumors (TN vs non-TN; P=0.0052). By comparing the gene profiles of individual tumors with CMTC-3, a correlation coefficient (r) was calculated as an index reflecting its degree of similarity to the expression pattern of CMTC-3 tumors. The two graphs show the relationship between r value and pCR (C) in the 111 Her2+/TN cancers and (D) in all 248 cancers. pCR status (PCR is grey and no PCT is white), Her2+ status (lighter) and TN status (darker), respectively, are indicated by the bottom bars. AUC=area under the curve; CMTC=ClinicoMolecular Triad Classification; Her2=human epidermal growth factor receptor 2; pCR=pathological response; TN=triple-negative.

FIG. 5 shows the generation of gene expression profile for Her2+/TN phenotype in the training cohort (n=149). (A). First screening of Her2+/TN related genes. 44 Her2+/TN breast cancers were used as the group to distinguish the gene expression from the other 105 tumors. 1428 probes were selected at a level of the Bonferroni corrected P value less than 0.01. By using the 1428-probe set in a hierarchical clustering pattern, 39 tumors that were mostly Her2+/TN formed group 3 with two other subgroups emerging on the heat map, groups 1 and 2. (B) Second screening for the most differentially expressed genes between the three groups. By ANOVA test, 1349 probes were selected at a level of P value less than 0.001 among the three groups. A three-cluster pattern was apparent on the heat map based on hierarchical clustering analysis using the 1349-probe set. The tumors with Her2+/TN status were 2.4% (1/42) in group 1, 10.3% (7/68) in group 2 and 92.3% (36/39) in group 3. The bottom color bars: lighter, Her2+; darker, TN.

FIG. 6 shows the benefits of endocrine therapy (ET) in CMTC-1 ER+ breast cancers at different cancer stages. Kaplan-Meier analyses were used to compare relapse-free survivals between ET-treated and no treatment ER+ breast cancers in 155 stage I CMTC-1 cancers (A), and in 142 stage 2 or worse (stage II+) CMTC-1 cancers (B). The P values were determined by Log-rank test.

FIG. 7. Graphs demonstrating that the prognostic accuracy of CMTC compared to subytype alone: A: Her+ cancers versus Her−. HR 0.71. B: TN cancers versus non-TN, HR 1.43 C:Combining the two subtypes as a group (Her+/TN), HR 1.56, D: Prognosis by CMTC class; CMTC2 & CMTC3 do worse than CMTC1, HR>2 with extremely small P values.

FIG. 8. Schematic representation of method for classifying a remotely obtained sample.

Table 1: Clinical and pathological variables in ClinicoMolecular Triad Classification of breast cancer in training and validation cohorts

Table 2: Summary of patient information and tumor pathological data for the training cohort of 149 breast cancers. CMTC=ClinicoMolecular Triad Classification; EIC=extensive intraductal component; IDC=invasive ductal carcinoma; LVI=lymphovascular invasion; PTID=Patient's identity number; RIN=RNA integrity number.

Table 3: Summary of resource, platform, adjuvant treatment status and clinical end point of the microarray data sets used in this study. DMFS=distant metastasis-free survival; RFS=relapse-free survival.

Table 4: Summary of name, definition, platform and reference of the prognostic signatures used in this study and the overlapped genes between ClinicoMolecular Triad Classification and published independent breast cancer gene expression prognostic signatures. TGF=transforming growth factor.

Table 5: Univariate and multivariate analyses of standard clinicopathological parameters, 14 independent gene signatures and CMTC as prognostic indicators for relapse among 1,058 breast cancer patients without adjuvant therapy in the validation cohort. CI=confidence interval; CMTC=ClinicoMolecular Triad Classification; ER=estrogen receptor; ERGS=estrogen-regulated gene expression signature; ESGS=embryonic stem cell-like gene signature; Her2=human epidermal growth factor receptor 2; IGS=“invasiveness” gene signature; LN=lymph node status; PAM50=50-gene prediction analysis of microarray; SDPP=stroma-derived prognostic predictor; TGFβRII=transforming growth factor receptor type II; TN=triple-negative; WS=wound-response gene signature.

Table 6: Association between relapse-free survivals and Her2+/TN status. Fourteen gene signatures and CMTC in the seven hundred fifty-six ER+ breast cancer patients with or without ET. ER=estrogen receptor; ERGS=estrogen-regulated gene expression signature; ESGS=embryonic stem cell-like gene signature; PAM50=50-gene prediction analysis of microarray; SDPP=stroma-derived prognostic predictor; TGFβRII=transforming growth factor receptor type II; TN=triple-negative; WS=wound-response gene signature.

Table 7: Receiver operating characteristic analysis of the ability of independent gene expression signatures to predict pathological complete responses in breast cancer treated with neoadjuvant chemotherapy. CI=confidence interval; CMTC=ClinicoMolecular Triad Classification; ERGS=estrogen-regulated gene expression signature; ESGS=embryonic stem cell-like gene signature; Her2=human epidermal growth factor receptor 2; IGS=“invasiveness” gene signature; LumA=luminal A; LumB=luminal B; PAM50=50-gene prediction analysis of microarray; SDPP=stroma-derived prognostic predictor; TGFβRII=transforming growth factor receptor type II; TN=triple-negative; WS=wound-response gene signature.

Table 8: The prediction of pCRs in 248 breast cancer patients treated with neoadjuvant chemotherapy on the basis of CMTC and 14 independent prognostic gene expression signatures. CMTC=ClinicoMolecular Triad Classification; PAM50=50-gene prediction analysis of microarray; SDPP=stroma-derived prognostic predictor; TGFβRII=transforming growth factor β receptor type II; WS=wound-response gene signature.

Table 9: CMTC 828-probe set including Illumina probe ID, gene symbol and the corresponding centroid value among the three CMTC groups of 149 breast cancers in the training cohort. CMTC=ClinicoMolecular Triad Classification.

Table 10. CMTC classification is reproducible using different genome wide platforms comprising different subsets of the 803 genes described in Table 9.

### DETAILED DESCRIPTION OF THE DISCLOSURE

### I. Abbreviations

AUC=area under the curve; CMTC=ClinicoMolecular Triad Classification; ER=estrogen receptor; ET=endocrine therapy; FNAB=fine-needle aspiration biopsy; Her2=human epidermal growth factor receptor 2 (also known as ERBB2); IFN=interferon; NPV=negative predictive value; pCR=pathological response; PI3K=phosphatidylinositol 3-knase; PPV=positive predictive value; PR=progesterone receptor; RIN=RNA integrity number; ROC=receiver operating characteristic analysis; RT-PCR=reverse transcriptase polymerase chain reaction; TN=triple-negative (ER−/PR−/Her2−).

### II. Definitions

The term “classifying” as used herein refers to assigning, to a class or kind, an unclassified item. A “class” or “group” then being a grouping of items, based on one or more characteristics, attributes, properties, qualities, effects, parameters, etc., which they have in common, for the purpose of classifying them according to an established system or scheme. For example, subjects having a subject expression profile similar to a CMTC-3 reference expression profile, fall within in a class CMTC-3 having poor outcome.

The term “Clinicomolecular Triad Classification” or “CMTC” as used herein means a three class breast cancer classification scheme which classifies subjects with breast cancer into one of the three classes according to the similarity of gene expression profiles of a plurality of CMTC genes to one or more reference CMTC profiles. The CMTC genes were identified by grouping TN and Her2+ breast cancers which have the worst prognosis into 1 group. Hierarchal clustering treating the TN and Her2+ breast cancers as one group, divided breast cancers into three groups that are compatible with current treatment strategies. Any plurality of genes (e.g. any number and any set of genes) that classifies breast cancer into three groups that are compatible with or correspond to current clinical treatment groups, can be used. For example the CMTC genes were identified by grouping TN and Her2+ breast cancers which have the worst prognosis into 1 group. Hierarchal clustering treating the TN and Her2+ breast cancers as one group, divided breast cancers into three groups that are compatible with current treatment strategies. The classification based on treatment of TN and Her2+ as one group was better than either of these groups alone or combining their prognostic accuracy as demonstrated in FIG. 7. Further various subsets of genes identified and listed in Table 9, can be used with the same classification outcome (see Table 10). For example at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes, for example at least 200, at least 300, at least 400, at least 500, at least 600, at least 700, at least 800 or 803 of the genes listed in Table 9 using a correlation method. The classes are identified as CMTC-1, CMTC-2 and CMTC-3 wherein CMTC-3 includes the majority of patients with Her2+/TN tumours and have poor prognosis. CMTC-1 includes the majority of estrogen receptor positive low proliferation patients and have a good prognosis. CMTC-2 includes patients who are estrogen receptor positive with high proliferation and have poor prognosis. The CMTC classes are clinically treatment relevant and the treatment recommended can be selected according to the class. For example, CMTC-1 patients in in general can be treated with surgery and tamoxifen alone, CMTC-2 patients will require additional treatments, including chemotherapy in addition to tamoxifen; other biologics can be prescribed based on the activities of additional oncogenic pathways and neo-adjuvant chemotherapy should be considered for CMTC-3 tumours (e.g. having an expression profile similar to triple negative and HER2+subjects) with addition of trastuzumab in those that show activation of the HER2 pathway.

The term “CMTC-1” refers to a class of subjects that are expected to have a good outcome, have typically an ER+ low proliferation breast cancer profile and who have an expression profile that comprises for a plurality of probes, the greatest similarity to the CMTC-1 profile, compared to the CMTC-2 profile and/or the CMTC-3 profiles for example as provided in Table 9. Table 9 provides for each probe the centroid value for each of CMTC-1, CMTC-2 and CMTC-3 classes. A negative centroid value is indicative of a relative average decrease and a positive centroid value is indicative of a relative average increase.

The term “CMTC-3” refers to a class of subjects that are expected to have a poor outcome, are typically Her2+ and/or TN and who have an expression profile that comprises for a plurality of probes, the greatest similarity to the CMTC-3 profile, compared to the CMTC-2 profile and/or the CMTC-1 profiles for example as provided in Table 9. Table 9 provides for each probe the centroid value for each of CMTC-1, CMTC-2 and CMTC-3 classes. A negative centroid value is indicative of a relative average decrease and a positive centroid value is indicative of a relative average increase.

The term “CMTC-2” refers to a class of subjects that are expected to have a poor outcome, have typically an ER+ high proliferation breast cancer profile and who have an expression profile that comprises for a plurality of probes, the greatest similarity to the CMTC-2 profile, compared to the CMTC-1 profile and/or the CMTC-3 profiles provided in Table 9. Table 9 provides for each probe the centroid value for each of CMTC-1, CMTC-2 and CMTC-3 classes. A negative centroid value is indicative of a relative average decrease and a positive centroid value is indicative of a relative average increase. CMTC-2 and CMTC-3 although both exhibit poor prognosis they differ from each other because their treatment strategies are different and they have different gene profiles and pathway patterns from each other. The term “CMTC genes” as used herein refers to a plurality of genes, for example at least 200, at least 300 genes, at least 400 genes, at least 500 genes, at least 600 genes, at least 700, or at least 800 genes, optionally the genes or a subset thereof listed in Table 9, for example any combination of Table 9 genes comprising at least 200, at least 300, at least 400, at least 500, at least 600, at least 700, at least 800 or 803 genes or any number between 200 and 803. Any subset of 803-genes in Table 9 that classifies BCs into the three clinical treatment groups (triad) based on where molecular subtypes (ie. TN and Her2+) are grouped into one, and by nature of its biological relevance, divides all BCs into the three groups that are compatible to current treatment strategies can be used. As shown in Table 10 various subsets of Table 9 genes can be used. For example only 529 genes in Affymetrix U133A overlapped with the 803 CMTC genes in the analysis described in Examples 1 and 2. For example, the genes can be at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes in Table 9. The genes can for example be any set of genes that are differentially expressed in TN and Her2+ cancers compared to non TN and Her2−cancers and which identify 3 classes using clustering analysis. Genome wide platforms such as Illumina, Affymetrix and Agilent which comprise a large number of genes can be used. For example, the initial experiments described herein were performed using Illumina HumanRef-8 v2 Expression BeadChips. The various platforms analyses (see for example Table 3 and 10 included only a subset of the genes listed in Table 9, yet the gene expression profiles were sufficient to predict a CMTC class that correlated with greater prognostic accuracy.

As used herein “prognosis” refers to an indication of the likelihood of a particular clinical outcome e.g. the resulting course of disease, for example, an indication of likelihood of survival or death due to disease within a fixed time period and/or relative to another class, and includes a “good prognosis” and a “poor prognosis”.

As used herein, “good prognosis” indicates that the subject is expected to survive without recurrence for a set time period, for example five years from initial diagnosis of breast cancer and/or have increased survival relative to the average for poor prognosis patients (e.g. untreated CMTC-3 and CMTC-2 profile patients). For example, CMTC-1 classified subjects typically having reduced recurrence within a predetermined period from initial diagnosis of breast cancer compared to CMTC-2 and CMTC-3 classified subjects (see for example FIG. 2B where recurrence for CMTC-1 in the first 5 years is less than 10% whereas recurrence in CMTC-2 and CMTC-3 for the same time period is about 35%) and/or having ER+ low proliferating breast cancer.

The term an “increased likelihood of survival”, as used herein means an increased likelihood or risk of longer survival relative to a subject relative to for example the median outcome for the particular cancer and/or relative to the average for poor prognosis patients (e.g. untreated CMTC-3 and CMTC-2 profile patients). Examples of expressions of risk include but are not limited to, odds, probability, odds ratio, p-values, attributable risk, relative frequency, positive predictive value, negative predictive value, and relative risk.

As used herein, “poor prognosis” indicates that the subject is expected to die due to disease within a set time period, for example five years of initial diagnosis and/or have decreased survival relative to the average for good prognosis patients (e.g. CMTC-1 profile patients). For example CMTC-2 and CMTC-3 classified subjects typically have increased recurrence within a predetermined period from initial diagnosis of breast cancer compared to CMTC-1 classified subjects (see for example FIG. 2B where recurrence for CMTC-1 in the first 5 years is less than 10% whereas recurrence in CMTC-2 and CMTC-3 for the same time period is about 35%). Poor prognosis patients can exhibit an ER+ high proliferating breast cancer profile and/or a TN/HER2+ breast cancer profile.

The term a “decreased likelihood of survival”, as used herein means an increased risk of shorter survival relative to for example the median outcome for the particular cancer and/or relative to the average for good prognosis patients. For example, increased expression of five or more genes in the gene signatures described herein can be prognostic of decreased likelihood of survival. The increased risk for example may be relative or absolute and may be expressed qualitatively or quantitatively. Examples of expressions of risk include but are not limited to, odds, probability, odds ratio, p-values, attributable risk, relative frequency, positive predictive value, negative predictive value, and relative risk.

The term “expression level” as used herein in reference to a gene for example a gene in Table 9, refers to a quantity of nucleic acid gene product (e.g. transcript) detectable or measurable in a breast cancer cell sample from a subject and/or control population (e.g. an average, median, error weighted etc. level). The expression level of a gene in a reference profile can also be referred to as a “reference level”.

The term “measuring” as used herein refers to assessing the presence, absence, quantity or amount (which can be an effective amount) of either a given substance within a clinical or subject-derived sample, including the derivation of qualitative or quantitative concentration levels of such substances, or otherwise evaluating the values (e.g. for similarity to expression levels in a reference expression profile) or categorization of a subject's clinical parameters.

The term “expression profile” as used herein refers to, for a plurality (e.g. at least 200) genes optionally at least 200 genes listed in Table 9 associated with CMTC class, gene transcript (e.g. mRNA) levels in a breast cancer cell sample from a subject.

The term “determining an expression profile” or “determining a subject expression profile” as used in reference to a gene expression level means the application of a gene specific reagent such as a probe or primer and/or a method to a sample, for example a breast cancer cell sample of the subject and/or a control sample or control samples (e.g. from patients with known prognosis), for ascertaining or measuring quantitatively, semi-quantitatively or qualitatively the amount of a gene expression, for example the amount of mRNA. For example, a level of gene expression can be determined by a number of methods including for example, hybridization and PCR protocols where a probe or primer or primer set are used to ascertain the amount of mRNA nucleic acid, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in optionally in fixed optionally formalin-fixed, paraffin-embedded (FFPE) tissue samples or cells, where expression level of a plurality of genes can be accurately determined. This technology is currently offered by the QuantiGene®ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “digital molecular barcoding technology” as used herein refers to a digital technology that is based on direct multiplexed measurement of gene expression that utilizes color-coded molecular barcodes, and can include for example NanostringnCounter™. For example, in such a method each color-coded barcode is attached to a target-specific probe, for example about 50 bases to about 100 bases or any number between 50 and 100 in length that hybridizes to a gene of interest. Two probes are used to hybridize to mRNA transcripts of interest: a reporter probe that carries the color signal and a capture probe that allows the probe-target complex to be immobilized for data collection. Once the probes are hybridized, excess probes are removed and detected. For example, probe-target complexes can be immobilized on a substrate for data collection, for example an nCounter™ Cartridge and analysed for example in a Digital Analyzer such that for example color codes are counted and tabulated for each target molecule.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, hybridization in 6.0× sodium chloride/sodium citrate (SSC) at about 45° C., followed by a wash of 2.0×SSC at 50° C. may be employed.

In methods employing commercial microarray platforms the hybridization conditions vary according to the manufacturer's protocol. For example as described below, DNA microarray analyses using an Illumina HumanRef-8 v2 Expression BeadChips hybridization can be performed according to the Illumina Whole-Genome Gene Expression direct hybridization assay protocols (Illumina Inc, San Diego, Calif., USA). Labeled cRNA can be hybridized to Illumina HumanRef-8 v2 Expression BeadChips (Illumina Inc.) overnight at 58° C. After washing, signals can be developed with streptavidin-Cy3, and scanned using the BeadArray Reader and processed using BeadStudio software obtained from Illumina.

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand.

The term “isolated nucleic acid” as used herein refers to a nucleic acid substantially free of cellular material or culture medium when produced by recombinant DNA techniques, or chemical precursors, or other chemicals when chemically synthesized.

The term “primer” as used herein refers to a polynucleotide, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “probe” as used herein refers to a nucleic acid sequence that will hybridize to a nucleic acid target sequence. In one example, the probe hybridizes to a signature gene RNA or a nucleic acid sequence complementary to the signature gene RNA. The length of probe that is optimal can depend for example, on hybridization conditions and the sequences of the probe and nucleic acid target sequence. The probe can be for example, at least 15, at least 20, at least 25, at least 50, at least 75, at least 100, at least 150, at least 200, at least 250, at least 400, at least 500 or more nucleotides in length.

A person skilled in the art would recognize that “all or part of” a particular probe or primer can be used as long as the portion is sufficient for example in the case a probe, to specifically hybridize to the intended target and in the case of a primer, sufficient to prime amplification of the intended template.

The term “reference expression profile” used interchangeably with “reference profile” as used herein refers to a suitable comparison profile associated with a CMTC class that comprises the expression levels (e.g. average expression levels associated with a class) of a plurality of genes of for example 200 or more genes for example at least 200 genes selected optionally from the genes listed in Table 9, derived as described elsewhere from expression profile hierarchal clustering of breast cancers from patients with TN/Her2+ breast cancer. For example reference expression profiles comprising a plurality of genes and centroid values associated with CMTC-1, CMTC-2, CMTC-3 can be derived as described herein for example in Examples 1 and 2. As shown, hierarchal clustering treating the TN and Her2+ breast cancers as one group, divided breast cancers into three groups that are compatible with current treatment strategies. Accordingly any plurality of genes that produces the triad clustering can be used. As shown here, a plurality of the genes listed in Table 9 can be used (see for example Table 10). Accordingly combinations of genes, including any combination of genes from Table 9, that classifies breast cancers into the three clinical treatment groups (triad) based on hierarchal clustering of the TN and Her2+molecular subtype, can be used. Table 9 provides the centroid value for each probe for each CMTC class and whether expression is decreased (negative value) or increased (positive value). The centroid value can be calculated for genes of other gene sets. Accordingly, the reference profile can comprise centroid values for a plurality of genes against which a subject expression is compared to classify the subject. For example, a “CMTC-1 reference profile” comprises the expression levels of said plurality of genes that are average mRNA expression levels in breast cancer cells of a plurality of breast cancer patients determined to fall within a CMTC-1 class, said plurality comprising optionally at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and/or their centroid expression values provided in Table 9. Similarly a “CMTC-2 reference profile” comprises the expression levels of said plurality of genes that are average mRNA expression levels in breast cancer cells of a plurality of breast cancer patients determined to fall within a CMTC-2 class, said plurality comprising optionally at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and/or their centroid expression values provided in Table 9. Further, a “CMTC-3 reference profile” comprises the expression levels of said plurality of genes that are average mRNA expression levels in breast cancer cells of a plurality of breast cancer patients determined to fall within a CMTC-3 class, said plurality comprising optionally of at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and/or their centroid expression values provided in Table 9.

It will be understood that “remote” herein refers to a location that is not the same or proximate the location where the CMTC classification is performed.

The term “sample” as used herein refers to any breast biological fluid, breast cell or breast tissue, such as a fine needle aspirate biopsy, or fraction thereof from a subject who has or is suspected of having breast cancer that can be assessed for gene expression products, including for example an isolated RNA fraction, optionally mRNA for nucleic acid biomarker determinations. The sample is preferably fresh tissue and/or cells and can be for example fresh tissue, frozen cells/tissue and optionally fixed cells/where expression levels for a plurality of genes can be accurately determined. The sample can for example be a test sample which is a patient sample to be tested or a control sample (or samples) which is a sample or samples with known outcome or ER/PR/Her2+ status used for comparison.

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, word_length=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “similar” in the context of a gene expression level as used herein refers to a subject gene expression level that falls within the range of levels associated with a particular class for example associated with CMTC class. Accordingly, “detecting a similarity” refers to detecting a gene expression level that falls within the range of levels associated with a particular class and/or prognosis. For example, the method for assessing similarity can comprise a nearest expression centroid method or other methods. In the context of a reference profile, “similar” refers to the CMTC reference profile that shows a number of identities and/or degree of changes with the subject expression profile.

The term “most similar” in the context of a reference profile refers to a reference profile that shows the greatest number of identities and/or degree of changes with the subject expression profile.

The term “specifically binds” as used herein refers to a binding reaction that is determinative of the presence of the gene expression product (e.g. mRNA, cDNA etc) often in a heterogeneous population of macromolecules. For example, a probe that specifically binds refers to the specified probe under hybridization conditions such as stringent hybridization conditions, binds to a particular gene sequence at least 1.5, at least 2 at least 3, or at least 5 times background.

The term “subject” or “test subject” or “patient” as used herein refers to any member of the animal kingdom, preferably a human being.

The term “microarray” or “array” as used herein refers to an ordered set of probes fixed to a solid surface that permits analysis such as gene analysis of a set of genes. A DNA microarray refers to an ordered set of DNA fragments fixed to the solid surface. For example, the microarray can be a gene chip. Methods of detecting gene expression and determining gene expression levels using arrays are well known in the art. Such methods are optionally automated.

The term “assay control” as used herein means a suitable assay control suitable according to the specific assay that is useful for determining an expression level of a Table 9 gene or set of genes. For kits for detecting RNA levels for example by hybridization, the assay control can comprise an oligonucleotide control, useful for example for detecting an internal control such as GAPDH for standardizing the amount of RNA in the sample and determining relative biomarker transcript levels. The assay can control can also include RNA from a cell line which can be used as a ‘baseline’ quality control in an assay, such as an array or PCR based method. The assay control can be internal to a particular assay. For example, commercial microarray platforms have built in internal assay controls. As an example, every array on each HumanRef-8 Expression BeadChip includes 775 bead types as controls.

The phrase “therapy” or “treatment” as used herein, refers to an approach aimed at obtaining beneficial or desired results, including clinical results and includes medical procedures and applications including for example chemotherapy, endocrine therapy, other pharmaceutical interventions, surgery, radiotherapy and naturopathic interventions as well as test treatments for treating breast cancer. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “suitable treatment” as used herein refers to a treatment suitable according to the determined CMTC class. For example, a suitable treatment for a subject with a poor prognosis can include a more aggressive treatment, for example, in the case of subjects identified as CMTC-3 this can include neoadjuvant chemotherapy and surgery. CMTC-3 patients would not benefit from endocrine therapy as they are ER−. A suitable treatment for CMTC-1 subjects, can include for example endocrine therapy as endocrine therapy is a suitable treatment for ER+ cancers. Patients identified as CMTC-2 which have ER+ cancers that are high proliferating are suitably treated with endocrine therapy and chemotherapy.

The term “breast cancer” as used herein includes “breast tumour” which implies a breast cancer tumour in the breast.

As used herein “a user interface device” or “user interfaced” refers to a hardware component or system of components that allows an individual to interact with a computer e.g. input data, or other electronic information system, and includes without limitation command line interfaces and graphical user interfaces.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and the include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### III. Methods and Products

It is demonstrated herein that patients can be classified using ClinicoMolecular Triad Classification (CMTC) and that such classification correlates with clinical outcome in breast cancer patients. The CMTC is an independent classifier and classifies patients into one of three classes: CMTC-1, CMTC-2 and CMTC-3. Subjects classified as CMTC-1 have good prognosis and subjects classified as CMTC-2 and CMTC-3 have poor prognosis (see for example FIG. 2B). The CMTC-3 profile was derived from tumours with Her2+/triple negative (TN) gene expression profiles and CMTC-3 comprises the majority of TN and Her2+classified subjects. The CMTC-1 classified subjects are typically ER+ and exhibit a low proliferation profile whereas CMTC-2 classified patients have a profile that shares similarities with CMTC-1 and CMTC-3. CMTC-2 includes for example, subjects afflicted with breast cancers which are ER+ and express a high proliferation profile. Although CMTC-2 and CMTC-3 classified subjects have poor outcomes, the treatment options for CMTC-2 and CMTC-3 may be different. For example, it is demonstrated herein that CMTC-2 patients do not benefit from endocrine treatment alone even though they are ER+. CMTC-2 may benefit with treatment regimens that comprise both an endocrine treatment component and chemotherapy. CMTC-2 and CMTC-3 are also phenotypically different as they had different gene profiles and pathway patterns which can provide further insight into treatment options.

It is demonstrated herein that that the CMTC classification based on a combination expression profile of Her2+ and TN negative breast cancers is superior to assessing clinical Her2/TN status alone in predicting recurrence and treatment response. As disclosed below in the Examples, the CMTC predicted recurrence and treatment response better than all pathological parameters and other prognostic signatures. FIG. 7 also demonstrates that the prognostic accuracy of CMTC is better than classifications based on subytype alone. For example FIG. 7A shows that Her+ cancers do worse than Her−exhibiting a hazard ratio (HR) of 0.71. FIG. 7B shows that TN cancers do worse than non-TN with a HR of 1.43. FIG. 7C shows that combining the individual two subtypes as a group (Her+/TN) results in a HR of 1.56. FIG. 7D demonstrates that classification based on CMTC is more accurate—both CMTC-2 and CMTC-3 do worse than CMTC1, HR>2 and this differentiation is highly significant as indicated by the small P values associated therewith.

As shown in FIG. 7 the CMTC molecular profile is better than simply grouping TN and Her2 subtypes together, as CMTC represent the natural division of BC based on the biological processes involved reflected on the pathways analyses (e.g. as demonstrated by the analysis of the 19 oncogenic pathways described in the Examples).

Additionally prognosis can be made at the time of diagnosis (e.g. at the time of biopsy), allowing for treatment planning. The CMTC is based on genome wide gene expression levels. It is demonstrated herein that a variety of genome wide microarray platforms can be used making the CMTC flexible and amenable to a wide variety of platforms.

It can also be combined with other gene signatures such as those described herein. For example, Table 4 showed that by using genome wide gene profiles, the scores of other gene signatures can be determined even though these other gene signatures were originally derived from other multigene platforms (not all were microarray).

As mentioned, the CMTC classes can also be combined with oncogenic pathway analysis as described in the Examples.

As described herein, CMTC-3 is a reference profile that clusters based on the expression levels of a group of breast cancer tumours that are Her2+ and TN. Her2+ and TN breast cancers were analyzed as one group, unlike prior art methods. Hierarchal clustering treating the TN and Her2+ breast cancers as one group, divided breast cancers into three groups that are compatible with current treatment strategies, which is very useful. As a result the triad classification allows for example, analysis of the activation of oncogenic pathways and other cellular pathways for example through addition of other signatures in the clinically relevant treatment groups such that current treatments can be adapted or supplemented according to the further classification.

Accordingly an aspect of the disclosure includes a method for classifying a subject afflicted with breast cancer according to a ClinicoMolecular Triad Classification (CMTC)-1, CMTC-2 or CMTC-3 class, the method comprising:
(i) determining a subject expression profile, said subject expression profile comprising the mRNA expression levels of a plurality of genes that classifies breast cancer into three groups by which the molecular subtypes TN and Her2+are grouped into one class, in a breast cancer cell sample taken from said subject;
(ii) calculating a measure of similarity between said subject expression profile, and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients; and
(iii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

The plurality of genes can for example be any set of genes that produces the triad classification, which can be determined as described in the examples. As shown herein for example in Table 10, different gene sets can be used. The plurality of genes and reference profiles for the CMTC classes as described herein are identified by identifying the genes and their expression levels that cluster TN and Her2+ breast cancers. Clustering on the basis of TN and Her2+ cancers as one group, results in the triad division described herein. Each class can be considered a treatment class as the responses to treatment between these classes differ.

The plurality of genes can also comprise a subset of genes in Table 9. As mentioned subsets thereof as shown in Table 10 can be used to classify breast cancers according to CMTC classes.

Similarity is assessed in certain embodiments, by calculating one or more measures of similarity between a subject expression profile, comprised of the expression levels of a plurality of genes, and a reference profile (e.g. comprising expression levels (such as average, median etc. expression levels), for the plurality of genes in a group of patients with known outcome and/or known ER/PR/HER2 status). For example, a correlation coefficient can be calculated with one or more CMTC-1, CMTC-2 or CMTC-3 reference profiles and the highest correlation coefficient identifying the class identified for the subject.

In an embodiment, the method for classifying a subject afflicted with breast cancer according to a CMTC-1, CMTC-2 or CMTC-3 class, comprises: (i) calculating a measure of similarity between a subject expression profile, said subject expression profile comprising the mRNA expression levels of a plurality of genes, the plurality comprising at least 200, at least 300, at least 400, at least 500, at least 600, at least 700, or at least 800, optionally at least 200, at least 300, at least 400, at least 500, at least 600, at least 700, or at least 800, or all 803 of the genes listed in Table 9 in a breast cancer cell sample taken from said subject and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients and (ii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

In an embodiment, the similarity is assessed by calculating a correlation coefficient between the subject expression profiles and one or more of CMTC-1, CMTC-2 and CMTC-reference profiles, and the subject is classified as falling in the class that has the highest correlation coefficient.

The CMTC reference profiles can for example be de novo generated and alternate pluralities of genes identified and centroid values calculated using the methods described herein or cab be based on the genes and values provided in Table 9. The CMTC-1, CMTC-2, and/or CMTC-3 reference profiles can for example be ne novo generated by selecting a plurality of genes, for example at least 200, at least 300, at least 400, at least 500, at least 600, at least 700, or at least 800, genes that using hierarchal clustering treating the TN and Her2+ breast cancers as one group, divided breast cancers into three groups that are compatible with current treatment strategies. The centroid expression value for each of the plurality of genes can be determined and used to classify subjects based on their expression profiles. For example, any subset of 803-genes in Table 9 that, by hierarchal clustering treating TN and Her2+ breast cancers as one group, divides breast cancers into three groups classifies breast cancers can be used.

In an embodiment, the method for classifying a subject afflicted with breast cancer according to a ClinicoMolecular Triad Classification (CMTC)-1, CMTC-2 or CMTC-3 class, comprises: (i) calculating a first measure of similarity between a subject expression profile, said subject expression profile comprising the mRNA expression levels of a plurality of genes comprising optionally comprising at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes selected from Table 9 in a breast cancer cell sample taken from said subject and a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancers; calculating a second measure of similarity between said subject expression profile and a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; calculating a third measure of similarity between said subject expression profile and a CMTC-3 reference profile, said CMTC-3 reference profile comprising expression levels of said plurality of genes that are average mRNA expression levels of said respective genes in breast cancer cells of a plurality of triple negative and HER2+ breast cancer patients and (ii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

In an embodiment, the subject is classified as falling in said CMTC-1 class if said subject expression profile has a higher similarity to said CMTC-1 reference profile than to said CMTC-2 and/or CMTC-3 reference profile, said subject is classified as falling within said CMTC-2 class if said subject expression profile has a higher similarity to said CMTC-2 reference profile than to said CMTC-1 and/or CMTC-3 reference profile, or said subject is classified as falling in said CMTC-3 class if said subject expression profile has a higher similarity to said CMTC-3 reference profile than to said CMTC-1 and/or CMTC-2 reference profile.

In an embodiment, the CMTC reference profiles comprise for at least 200, at least 300, at least 400, at least 500, at least 600, at least 700, at least 800 or all 803 genes in Table 9, the respective centroid values listed in Table 9. In another embodiment, the CMTC reference profiles comprise at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and their respective centroid values listed in Table 9.

In embodiments comprising one or more measures of similarity such as a first and/or second and/or third measure of similarity, said first measure of similarity can be represented by a correlation coefficient between said subject expression profile and said CMTC-1 reference profile. and said second measure of similarity can be represented by a correlation between said subject expression profile and said CMTC-2 reference profile and/or said third measure of similarity can be represented by a correlation coefficient between said subject expression profile and said CMTC-3 reference profile, wherein said highest correlation coefficient indicates the highest similarity and/or most similar CMTC profile.

Accordingly, in another embodiment, the method comprises: (i) calculating a first measure of similarity between a subject expression profile, said subject expression profile comprising the mRNA expression levels of a plurality of genes comprising at least 25%, at least 30%, at least 35%, at least 40%, or at least 50% of the genes listed in Table 9 in a breast cancer cell sample taken from said subject and a CMTC-1 reference profile, said CMTC-1 reference profile comprising expression levels of said plurality of genes that are average mRNA expression levels of said respective genes in breast cancer cells of a plurality of breast cancer patients having no recurrence within a predetermined period from initial diagnosis of breast cancer and/or having ER+ low proliferating breast cancer; ii) calculating a second measure of similarity between said subject expression profile and a CMTC-2 reference profile, said CMTC-2 reference profile comprising expression levels of said plurality of genes that are average mRNA expression levels of the respective genes in breast cancer cells of a plurality of breast cancer patients having recurrence within a predetermined period from initial diagnosis of breast cancer and/or ER+ high proliferating breast cancer; iii) calculating a third measure of similarity between said subject expression profile and a CMTC-3 reference profile, said CMTC-3 reference profile comprising expression levels of said plurality of genes that are average mRNA expression levels of the respective genes in breast cancer cells of a plurality of breast cancer patients having recurrence within a predetermined period from initial diagnosis of breast cancer and/or TN or HER2+ breast cancer; and iv) classifying said subject as falling in said CMTC-1 class if said subject expression profile has a higher similarity to said CMTC-1 reference profile than to said CMTC-2 or CMTC-3 reference profile, or classifying said subject as falling within said CMTC-2 class if said subject expression profile has a higher similarity to said CMTC-2 reference profile than to said CMTC-1 or CMTC-3 reference profile, or classifying said subject as falling in said CMTC-3 class if said subject expression profile has a higher similarity to said CMTC-3 reference profile than said CMTC-1 or CMTC-3 reference profile.

In an embodiment, the highest correlation coefficient (r) is used to classify the subject afflicted with breast cancer.

CMTC-1, CMTC-2 and CMTC-3 classes are associated with a prognosis, for example e.g. good prognosis, or poor prognosis or good prognosis (CMTC-1) and poor prognosis (CMTC-2 and CMTC-3), and the method can be used to provide the subject with a prognosis classification.

Accordingly in an embodiment, the disclosure provides a method for providing a subject afflicted with breast cancer with a prognosis classification, the method comprising: (i) calculating a measure of similarity between a subject expression profile, said subject expression profile comprising the mRNA expression levels of a plurality of genes comprising at least 200 genes, optionally at least 200 genes listed in Table 9 in a breast cancer cell sample taken from said subject and one or more of: a) a CMTC-1 reference profile, said CMTC-1 reference profile comprising expression levels of said plurality of genes that are average mRNA expression levels of said respective genes in breast cancer cells of a plurality of breast cancer patients having no recurrence within a predetermined period from initial diagnosis of breast cancer and/or having ER+ low proliferation breast cancer b) a CMTC-2 reference profile, said CMTC-2 reference profile comprising expression levels of said plurality of genes that are average mRNA expression levels of said respective genes in breast cancer cells of a plurality of breast cancer patients having recurrence within a predetermined period from initial diagnosis of breast cancer and/or having ER+ high proliferation breast cancer an CMTC-2 reference profile; and c) a CMTC-3 reference profile, said CMTC-3 reference profile comprising expression levels of said plurality of genes that are average mRNA expression levels of said respective genes in breast cancer cells of a plurality of breast cancer patients having recurrence within a predetermined period from initial diagnosis of breast cancer and/or having TN and/or HER2+ breast cancer; (ii) classifying said subject as having the poor prognosis if said subject expression profile is most similar to said CMTC-3 reference profile or said CMTC-2 reference profile, or classifying said subject as having said good prognosis if said subject expression profile is most similar to said CMTC-1 reference profile; and iii) providing said prognosis classification to the subject.

In another embodiment, said subject is classified as having a good prognosis if said subject expression profile has a higher similarity to said CMTC-1 reference profile than to said CMTC-3 reference profile and/or said CMTC-2 reference profile, or said subject is classified as having said poor prognosis if said subject expression profile has a higher similarity to said CMTC-3 reference profile or said CMTC-2 expression profile than to said CMTC-1 reference profile and/or.

For any of the embodiments described, the method can further comprise (iii) displaying; or outputting to a user interface device, a computer-readable storage medium, or a local or remote computer system, the classification produced by said classifying step (ii).

In another embodiment, the method described herein can comprise one or more computer implemented steps. For example, in an embodiment, the disclosure includes a computer-implemented method for classifying a subject afflicted with breast cancer according to prognosis comprising:

obtaining a subject expression profile; the subject expression profile comprising the mRNA expression levels of a plurality of genes comprising at least 200 genes, optionally at least 200 genes listed in Table 9 in a breast cancer cell sample taken from said subject;

comparing the subject expression profile to one or more reference expression profiles selected from a CMTC-1, CMTC-2 and CMTC-3 reference profiles, each reference profile comprising expression levels of said plurality of genes that are average mRNA expression levels of said respective genes in breast cancer cells of a plurality of breast cancer patients; and

classifying, on a computer, the subject as having a good prognosis, or a poor prognosis and/or falling within a CMTC-1, CMTC-2 or CMCT-3 class based on the similarity of the subject expression profile to the one or more reference profiles.

In embodiments described herein, the method can further comprise determining a subject expression profile. For example, the level of gene expression can be determined by a number of methods including for example, hybridization and PCR protocols where a probe or primer or primer set are used to ascertain the amount of mRNA nucleic acid, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

Suitable arrays include genome wide arrays, including for example Illumina HumanRef-8 v2 Expression BeadChips, Agilent and Affymetrix platforms such as those listed in Tables herein such as Table 10 and including such as Agilent Hu25K and Affimetrix U133 or any platform that includes probes for at least 70% of the genes identified by accession number in Table 9, the transcript sequences (e.g. cDNA or mRNA sequence) of which are incorporated herein by reference. For example, the array platform can include at least, 25%, 30%, 35%, 40%, 45%, 50%, 55%, 60%, 65%, 70% or more probes corresponding to the Illumina probes identified by number in Table 9 (e.g. corresponding including probes that are specific for the same gene), the probe sequences of which are incorporated herein by reference.

In yet another embodiment, the method of classifying a subject afflicted with breast cancer according to prognosis comprises:

determining a subject expression profile, said subject expression profile comprising the mRNA expression levels of a plurality of genes comprising at least 200 genes listed in Table 9 in a breast cancer cell sample taken from said subject;

comparing said subject expression profile with one or more of a CMTC-3 reference profile, said CMTC-3 reference profile comprising expression levels of said plurality of genes that are average mRNA expression levels of said respective genes in breast cancer cells of a plurality of breast cancer patients having recurrence within a predetermined period from initial diagnosis of breast cancer and/or having TN and/or HER2+ breast cancer; a CMTC-2 reference profile, said CMTC-2 reference profile comprising expression levels of said plurality of genes that are average mRNA expression levels of said respective genes in breast cancer cells of a plurality of breast cancer patients having ER+ high proliferating breast cancer; and a CMTC-1 reference profile, said CMTC-1 reference profile comprising expression levels of said plurality of genes that are average mRNA expression levels of said respective genes in breast cancer cells of a plurality of breast cancer patients having no recurrence within a predetermined period from initial diagnosis of breast cancer and/or having ER+ low proliferating breast cancer;

calculating one or more measures of similarity between said subject expression profile and said CMTC-3 reference profile, between said subject expression profile and said CMTC-1 reference profile and/or said subject expression profile and said CMTC-2 reference profiles;

classifying the subject as having a good prognosis, or a poor prognosis based on the subject expression profile similarity to the one or more reference profiles.

In an embodiment, determining a subject expression profile comprises hybridizing a nucleic acid fraction of said breast cancer sample from the subject with an array, said array comprising a plurality of probes for detecting the expression level of a plurality of genes, including a plurality of CMTC genes and measuring the level of gene expression for said plurality of genes.

In an embodiment, the method further comprises obtaining a breast cancer cell sample taken from said subject.

It is also demonstrated herein that the ClinicoMolecular Triad Classification correlates with the benefit to endocrine therapy. CMTC-1 patients, unlike CMTC-2 and CMTC-3 patients, benefitted from endocrine therapy (see for example FIGS. 3C and 3D). Subjects identified as having a CMTC-2 profile which are ER+, may benefit from combination chemotherapy and endocrine therapy.

It is also demonstrated herein that the ClinicoMolecular Triad Classification predicts complete pathological response to neoadjuvant therapy. CMTC-3 patients had an increased pathological complete response to neoadjuvant chemotherapy.

Accordingly the methods and products described can be used for example to identify treatments suitable according to the prognosis, accordingly a further embodiment comprises the step of providing a cancer treatment to the subject suitable with the prognosis and/or class determined according to a method described herein.

A further aspect includes a method for monitoring a response to a cancer treatment in a subject afflicted with breast cancer, comprising:

collecting a first breast cancer cell sample from the subject i) before the subject has received the cancer treatment and/or ii) during treatment and collecting a subsequent breast cancer cell sample from the subject after the subject has received at least one cancer treatment dose;

determining a first subject expression profile, said first subject expression profile comprising the mRNA expression levels of a plurality of genes of said first breast cancer cell sample and determining a second subject expression profile, said second subject expression profile comprising the mRNA expression levels of said plurality of genes of said subsequent breast cancer cell sample, said plurality of genes comprising at least 200 genes and optionally at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes listed in Table 9;

classifying said subject as having a good prognosis, or a poor prognosis or as falling in CMTC-1, CMTC-2 or CMTC-3 based on said first subject expression profile and classifying said subject as having a good prognosis, intermediate-poor prognosis or a poor prognosis or as falling in CMTC-1, CMTC-2 or CMTC-3based on said second subject expression profile according to a method of described herein;

and/or calculating a first sample subject expression profile score and a subsequent sample subject expression profile score;

wherein a lower subsequent sample expression profile score or better prognosis class compared to the first sample expression profile score is indicative of a positive response, and a higher subsequent sample expression profile score or worse class compared to said first sample subject expression profile score is indicative of a negative response.

A further aspect includes a method of treating a subject afflicted with breast cancer, comprising classifying said subject according to a method described herein, and providing a suitable cancer treatment to the subject in need thereof according to the class determined.

Also provided in an embodiment is use of a suitable treatment for treating a subject with breast cancer, wherein the treatment is selected according to the classification determined according to a method described herein.

In an embodiment, the plurality of genes comprises and/or is a plurality of CMTC genes.

In embodiments, said plurality of genes comprises at least 200, at least 250, at least 300, at least 350, at least 400, at least 450, at least 500, at least 550, at least 600, at least 650, at least 700, at least 750, at least 800 or 803 of the genes listed in Table 9.

In other embodiments, said plurality of genes comprises 201-250 genes, 251-300 genes, 301-350 genes, 351-400 genes, 401-450 genes, 451-500 genes, 501-550 genes, 551-600, 601-650 genes, 651-700, 701-750 genes, 751-800 genes of 801 to 803 genes of the genes listed in Table 9.

In an embodiment, the plurality of genes comprises at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and optionally at least 97%, at least 98%, at least 99% or at least 100% of the genes listed in Table 9. Preferably the greatest number of probes for detecting gene expression of genes listed in Table 9 are used. For example, if Illumina HumanRef-8 v2 Expression BeadChips are used, 100% of the genes can be assessed. Other platforms may include fewer than 100% genes. However, as demonstrated herein, the large number of genes analysed for expression allows the effect of gene inclusion variations among different microarray platforms to be minimized.

CMTC is compatible with the other major commercial platforms, such as Affymetrix and Agilent, since it allows for use of as many genes IDs that are compatible with the 803-genes in these other platform to classify the tumours. As demonstrated herein, CMTC remained reproducible in the 3-group separation (Triad) and also prognostic to the same degree using other platforms that comprised a subset of the genes listed in Table 9.

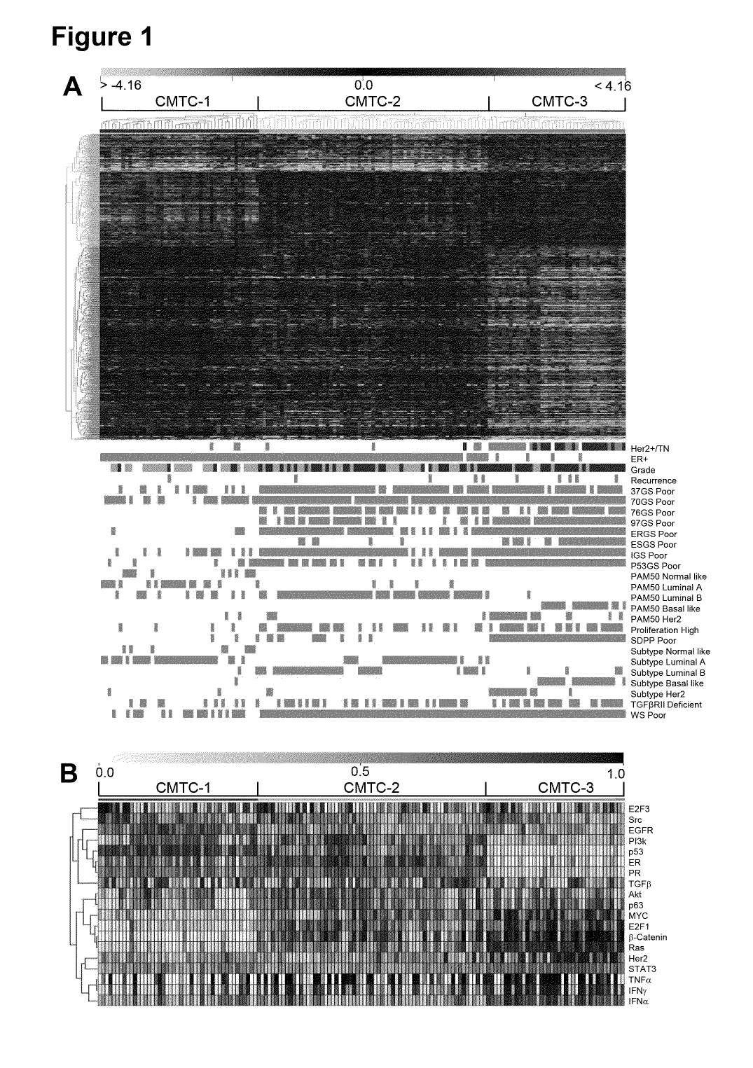

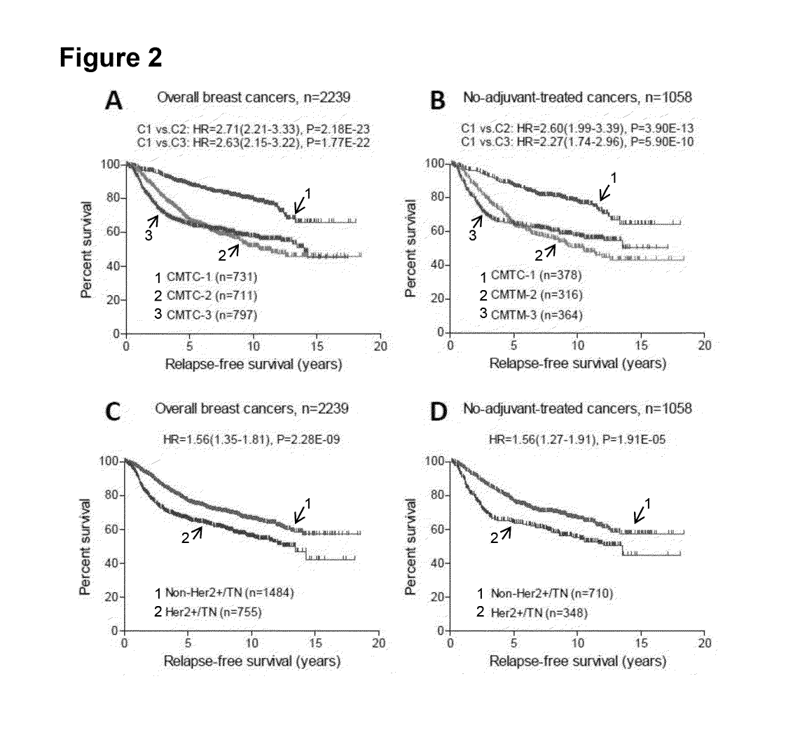

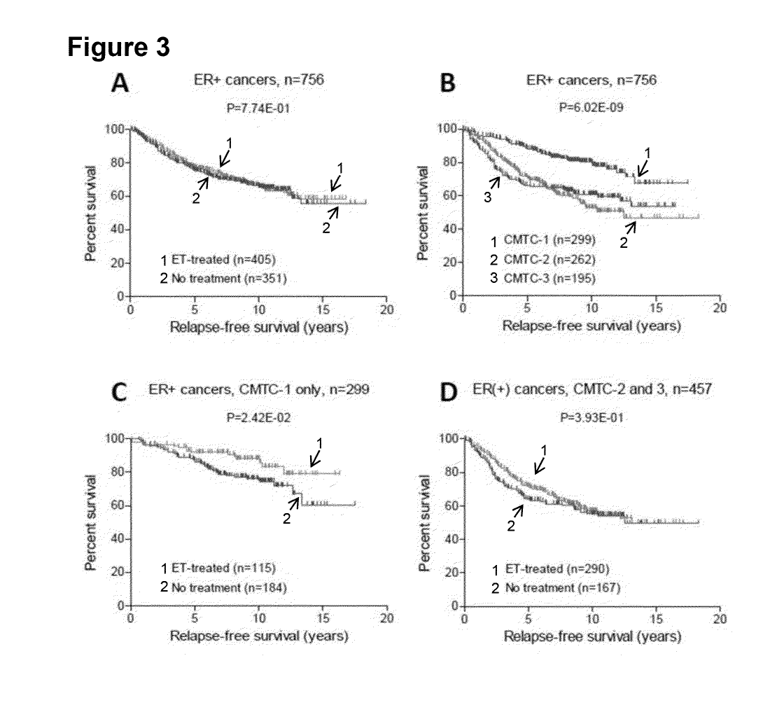

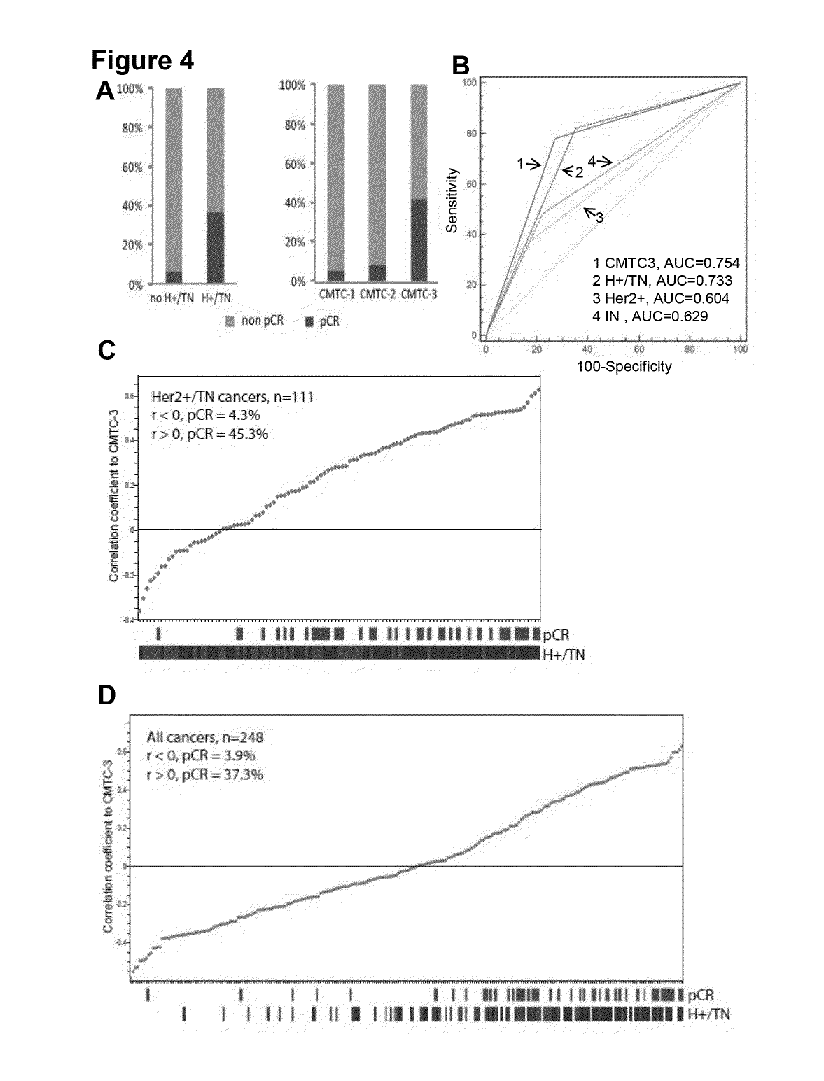

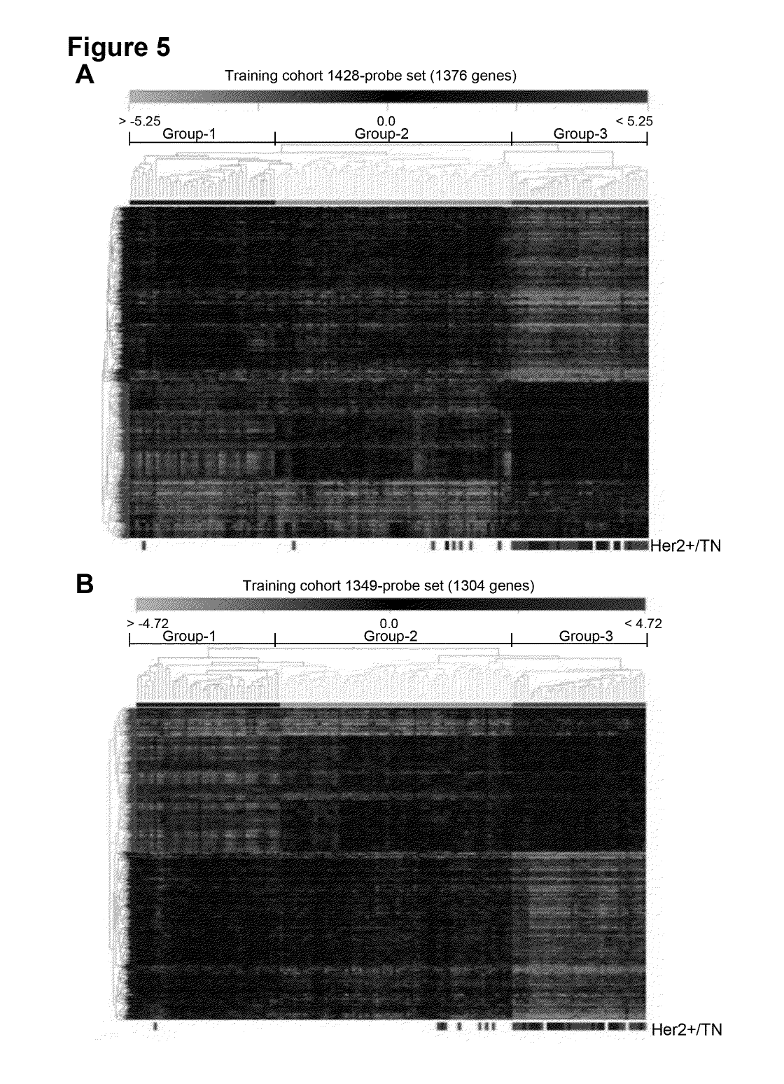

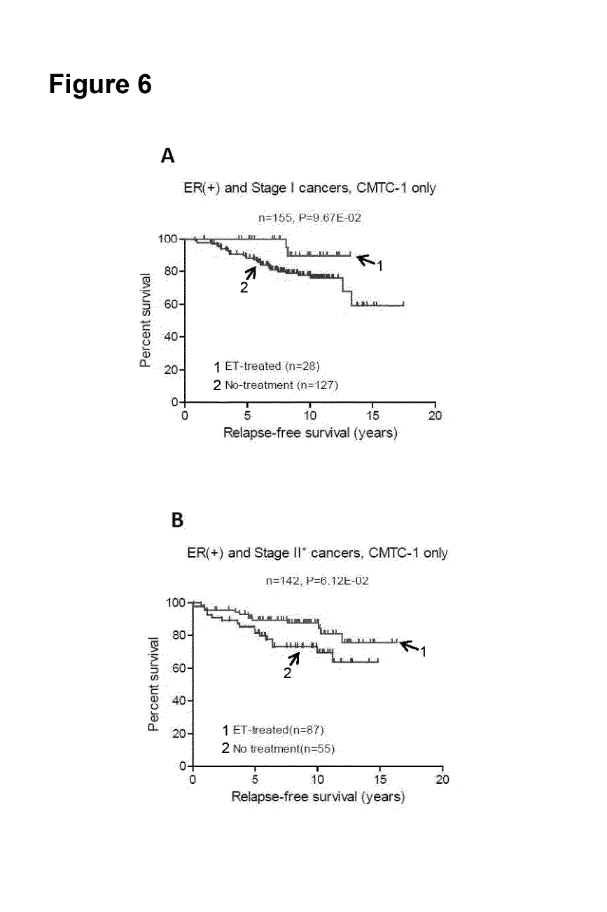

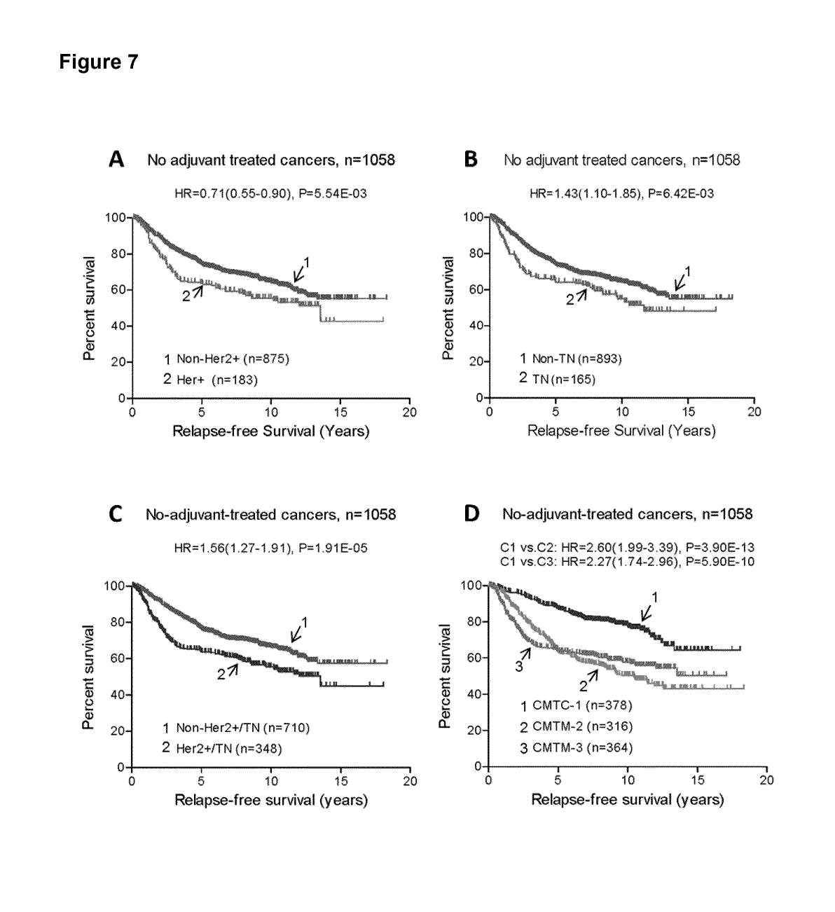

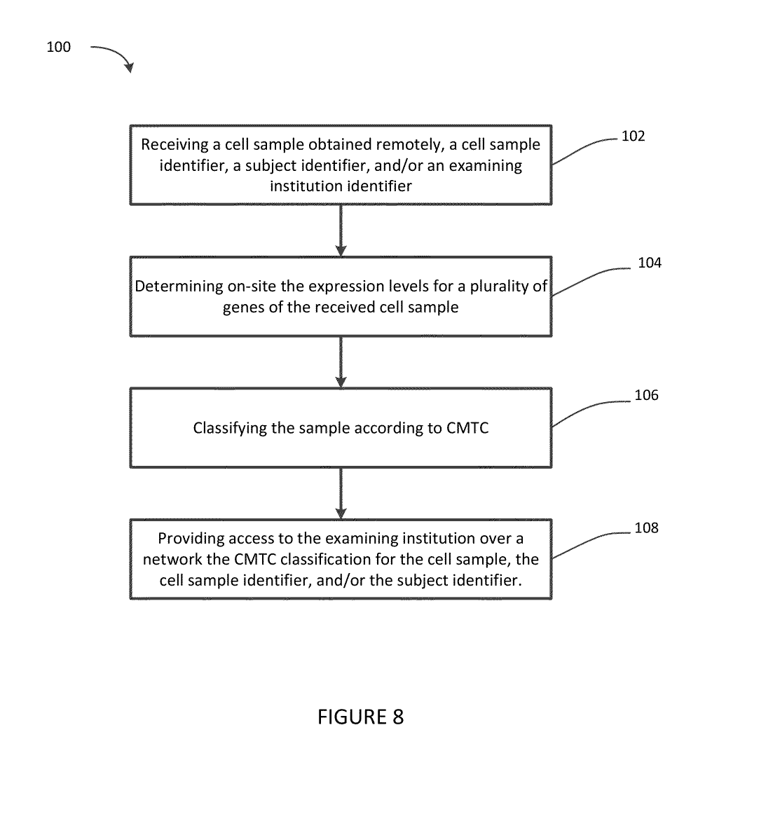

The genes provided in Table 9 include genes from across the genome. The versatility of a genome-wide approach allows the CMTC classification to be combined with other gene signatures and oncogenic pathways to provide a highly personalized “portfolios” that can for example be used to predict treatments based on the biological processes involved rather than individual biomarkers. In an embodiment, the CMTC classification is combined with one or more other gene classifiers. In an embodiment, the one or more other gene classifiers is selected from one of the classifiers described in FIG. 1B and/or Table 4.

It also enables multiplatform compatibility. For example, any standard commercial genome wide microarray can be used. As explained above, the gene set can be any gene set identified based on the consideration of TN and Her2+gene expression profiles as one group. In an embodiment, the genome wide array comprises at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes listed in Table 9.

In certain embodiments, said plurality of genes comprises each of the genes listed in Table 9.

The expression level of each gene in said subject expression profile can be for example a relative expression level of said gene in said breast cancer cell sample versus expression level of said gene in a reference pool.

In an embodiment, said reference pool is derived from a pool of breast cancer tumors derived from a plurality of individual breast cancer patients.

The expression level of each gene is optionally a log₂ ratio, for example a log₂ expression ratio of an intensity value to the average signal value for each transcript. The expression level can also be an average or median level, for example an average signal value. Accordingly in an embodiment, said relative expression level is represented as a log ratio.

In another embodiment, each expression level of said reference profile or said prognosis reference profile comprising expression levels of the plurality of genes is an error-weighted average.

In an embodiment, said predetermined period from initial diagnosis can for example be 1 year, 2 years, 3 years, 4 years, 5 years or 10 years. FIG. 2B for example shows the difference in recurrence of subjects identified as CMTC-1, CMTC-2 and CMTC-3.

In the methods described herein, each of said mRNA expression levels can be determined using one or more polynucleotide probes and/or one or more polynucleotide probe sets.

For example, the one or more polynucleotide probes and/or the one or more polynucleotide probe sets can be selected from the Illumina probes identified in Table 9. The polynucleotide probes described for example in Table 9, comprise sets of probes that are targeted to a particular gene expression product. Any, all or a subset of the probes listed for each gene can be used. Other gene transcript specific probes can also be used. The probe or probes are optionally immobilized, for example on an array.

In embodiments, the mRNA expression level is determined using an array and/or PCR method, optionally multiplex PCR.

In an embodiment where the method employs use of an array, the method comprises: (a) contacting first nucleic acids derived from mRNA of a breast cancer cell sample taken from said subject, and optionally a second nucleic acids derived from mRNA of two or more breast cancer cell samples from breast cancer patients who have recurrence within a predetermined period from initial diagnosis of breast cancer and/or known ER/PR/HER2 clinical status, with an array under conditions such that hybridization can occur, wherein the first nucleic acids are labeled with a first fluorescent label, and the optional second nucleic acids are labeled with e second fluorescent label, detecting at each of a plurality of discrete loci on said array a first fluorescent emission signal from said first nucleic acids and optionally a second fluorescent emission signal from said second nucleic acids that are bound to said array under said conditions, wherein said array comprises at least 200 of the genes listed in Table 9; (b) calculating a first measure of similarity between said first fluorescent emission signals and said second fluorescent emission signals across said at least 200 genes or calculating one or more measures of similarity between said first fluorescent emission signals and one or more reference profiles; (c) classifying said subject based on the similarity between said first fluorescent emission signals and said second fluorescent emission signals across said at least 200 genes or based on the similarity between said first fluorescent emission signals and said one or more reference profiles across said at least 200 genes (e.g. CMTC-1, CMTC-2 and/or CMTC-3) wherein said individual is classified as having a good prognosis if said subject expression profile has a low similarity to the CMTC-3 reference profile, as having an intermediate poor outcome if said subject expression profile has an intermediate similarity to the CMTC-3 reference profile or as having a poor outcome if said subject expression profile has a high similarity to a CMTC-3 reference profile or alternatively said individual is classified as having a a good prognsosis if said subject expression profile is most similar to a CMTC-1 reference profile an intermediate-poor prognosis if said subject expression profile is most similar to said CMTC-2 prognosis reference profile or a poor prognosis if said subject expression profile is most similar to said CMTC-3 reference profile; and (d) displaying; or outputting to a user interface device, a computer readable storage medium, or a local or remote computer system; the classification produced by said classifying step (c).

A further aspect includes a composition comprising a plurality of nucleic acid probes each comprising a polynucleotide sequence selected from the probe sequences identified by number in Table 9.

In an embodiment, the composition comprises at least 5-22, at least 23-44, at least 45-66, at least 67-88, at least 89-110, at least 111-132, at least 133-154, at least 155-176, at least 177-198, at least 199-220, at least 221-242, at least 243-264, at least 265-286, at least 287-308, at least 309-330, at least 331-352, at least 353-374, at least 375-396, at least 397-418, at least 419-440, at least 441-462 or at least 463-473 or up to 828 nucleic acid probes each comprising a polynucleotide sequence selected from the probe sequences identified by number in Table 9.

A further aspect include an array comprising for each gene in a plurality of genes, the plurality of genes comprising at least 200 of the genes listed in Table 9, one or more nucleic acid probes complementary and hybridizable to a coding sequence in the gene, for determining a classification according to a method described herein.

In certain embodiments, the array comprises nucleic acid probes for at least 5-22, at least 23-44, at least 45-66, at least 67-88, at least 89-110, at least 111-132, at least 133-154, at least 155-176, at least 177-198, at least 199-220, at least 221-242, at least 243-264, at least 265-286, at least 287-308, at least 309-330, at least 331-352, at least 353-374, at least 375-396, at least 397-418, at least 419-440, at least 441-462 or at least 463-473 or up to 803 of the genes listed in Table 9.

The array probes can for example comprise one or more polynucleotide probes selected from SEQ the probes identified by number in Table 9. For each gene the probes can comprise one or more of the gene specific probes provided in Table 9.

A further aspect comprises a method for classifying a remotely obtained breast cancer sample according to CMTC and providing access to the CMTC classification of the breast cancer cell sample, the method comprising:

receiving a remotely obtained breast cancer cell sample and a breast cancer cell sample identifier associated to the breast cancer cell sample;

determining on-site the expression levels for a plurality of genes of the received cell sample;

classifying the breast cancer cell sample according to CMTC;

providing access to the CMTC classification for the breast cancer cell sample.

In addition to or alternative to providing the CMTC classification, CMTC-1, CMTC-2, or CMTC-3, a prognosis may be provided.

In embodiments, the breast cancer cell sample may have been obtained at a medical institution that treats and examines subjects. For example, the medical institution may be a hospital or clinic. The breast cancer cell sample may be further identified by the subject or patient from whom the breast cancer cell sample was obtained. A subject identifier associated with the breast cancer cell sample may also be received.

For example, the breast cancer cell sample may also be identified by the examining institution where the breast cancer cell sample was obtained. The examining institution may refer to the hospital, clinic, department, or the subject's physician. The examining institution associated with the breast cancer cell sample may also be received.

It may be desirable to determine the expression levels of the genes on site because the remote location where the breast cancer cell sample was obtained may not have the required equipment. It may also become more efficient to provide a service at a single location for the determination of expression levels of the plurality of genes of breast cancer cell samples obtained at a number of remote locations.

In embodiments, the classifying of the breast cancer cell sample according to CMTC may be performed according to any of the methods described herein.

In embodiments, the CMTC classification for the breast cancer cell sample may be provided to the examining institution over a computer network, such as the Internet. For example, to ensure protection of sensitive information, the CMTC classification may be encrypted when it is provided to the examining institution. For example, the CMTC classification of the breast cancer cell sample may be provided via email.

In embodiments, the CMTC classification sample may be provided to more than one examining institutions for which the CMTC classification would be useful.

In embodiments, the CMTC classification for breast cancer cell sample may be stored in a database server as a cell sample entry. The CMTC classification can be stored in a breast cancer cell sample entry with one or more of the subject identifier, examining institution identifier and gene expression levels. The stored entries can be stored to be sortable and selectably retrieved by the subject identifier, examining institution identifier and gene expression levels. For example, method 100 may comprise an additional step performed between step 3 and 4, wherein the breast cancer cell sample information is accordingly stored.

It may be advantageous to store CMTC classification in the database for breast cancer cell sample for comparison or research purposes. For example, classifications for a plurality of breast cancer cell samples having the same subject identifier may be retrieved in order to show a subject's progress over time, such as over cancer treatment. Furthermore, the database may easily be used for research purposes by providing access to a plurality of CMTC classification results.

In embodiments where the CMTC classifications are stored in a database server, access to the classification may be provided to client devices across a network, such as the Internet. For example, a user of a client device must provide user credentials, such as a username and password, and the database server is configured to make available to the user all cell sample entries associated to the user.

In an embodiment, the method further comprises providing a kit for the remotely obtained breast cancer cell sample.

A further aspect comprises a kit for obtaining a breast cancer cell sample for determining a CMTC classification and/or prognosis in a subject afflicted with breast cancer according to a method described herein comprising one or more of:

a) a needle or other breast cancer cell sample obtainer;
b) tissue RNA preservative solution;
c) breast cancer cell sample identifier;
d) vial such as a cryovial; and
e) instructions.

The tissue RNA preservative solution for example may be any solution that inhibits degradation of RNA and/or stabilizes RNA in tissue specimen for transport and later isolation and testing.

The instructions for example include how to handle the sample, how to store the sample, how to label the sample, how to send the sample and how to receive the classification and/or diagnosis.

The needle can be any needle or syringe that is suitable for obtaining a biopsy. Similarly, the breast cancer cell obtainer can be any instrument useful for obtaining a biopsy.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Abstract

### Introduction:

When making treatment decisions, oncologists often stratify breast cancer (BC) into a low-risk group (low-grade estrogen receptor-positive (ER+)), an intermediate-risk group (high-grade ER+) and a high-risk group that includes Her2+ and triple-negative (TN) tumors (ER−/PR−/Her2−). None of the currently available gene signatures correlates to this clinical classification. In this study, we aimed to develop a test that is practical for oncologists and offers both molecular characterization of BC and improved prediction of prognosis and treatment response.

### Methods:

The molecular basis of such clinical practice was investigated by grouping Her2+ and TN BC together during clustering analyses of the genome-wide gene expression profiles of the training cohort, mostly derived from fine-needle aspiration biopsies (FNABs) of 149 consecutive evaluable BC. The analyses consistently divided these tumors into a three-cluster pattern, similarly to clinical risk stratification groups, that was reproducible in published microarray databases (n=2,487) annotated with clinical outcomes. The clinicopathological parameters of each of these three molecular groups were also similar to clinical classification.

### Results:

The low-risk group had good outcomes and benefited from endocrine therapy. Both the intermediate- and high-risk groups had poor outcomes, and their BC was resistant to endocrine therapy. The latter group demonstrated the highest rate of complete pathological response to neoadjuvant chemotherapy; the highest activities in Myc, E2F1, Ras, β-catenin and IFN-γ pathways;

and poor prognosis predicted by 14 independent prognostic signatures. On the basis of multivariate analysis, we found that this new gene signature, termed the “ClinicoMolecular Triad Classification” (CMTC), predicted recurrence and treatment response better than all pathological parameters and other prognostic signatures.

### Conclusions:

CMTC correlates well with current clinical classifications of BC and has the potential to be easily integrated into routine clinical practice. Using FNABs, CMTC can be determined at the time of diagnostic needle biopsies for tumors of all sizes. On the basis of using public databases as the validation cohort in our analyses, CMTC appeared to enable accurate treatment guidance, could be made available in preoperative settings and was applicable to all BC types independently of tumor size and receptor and nodal status. The unique oncogenic signaling pathway pattern of each CMTC group may provide guidance in the development of new treatment strategies. Further validation of CMTC requires prospective, randomized, controlled trials.

Further details are provided in Example 2

### Example 2

There is some indirect evidence that supports stratifying Her2+ and TN breast cancer into the same high-risk group. There is no significant difference in the clinical outcomes of patients with the basal-like and Her2+subtypes of breast cancer [5-7]. Even though there is no standard targeted systemic therapy for TN tumors [3,4,8], such as trastuzumab for Her2+ tumors [9], the rates of complete clinical response and complete pathological response (pCR) to neoadjuvant chemotherapies are also similar in both Her2+ and TN breast cancer [10-12]. Recently, investigators in both the CALGB 9840 trial [13] and the NSABP-B31 trial [14,15] reported responses of some Her2−breast cancers to trastuzumab and raised some controversies about the classification of breast cancer. Indirectly, these studies suggest that Her2+ breast cancer may not be as different from TN breast cancer as previously thought. Moreover, a relatively high proportion of TN tumors have genomic profiles similar to those of Her2+ tumors [16].

In the early 2000s, Perou and colleagues [6,7,17] reported the intrinsic gene expression profile that divides breast tumors into five or more molecular subtypes. More recently, on the basis of oncogenic pathway activity analysis, a more extensive classification with up to 18 subtypes for breast cancer was reported [18]. It remains a major challenge to use these molecular profiles to guide clinical treatment decisions [19] as they become increasingly complex for patients and clinicians alike and do not correlate with how breast cancer is clinically classified. On the other hand, many prognostic gene expression signatures that dichotomize selected patient populations into good and poor prognosis groups [20] lack the specificity to provide guidance on various treatment options.

In this study, we aimed to develop a molecular test that can be used preoperatively to guide treatment decisions, such as whether to initiate neoadjuvant therapy. For that reason, we decided to collect most of our clinical specimens by fine-needle aspiration biopsy (FNAB) taken from consecutive suspicious breast tumors at the time of clinical diagnostic core biopsy. Our study included relatively small breast cancers that had been routinely excluded in previous studies in which fresh surgical specimens or banked tissues were examined. After confirming the clinical diagnoses and the presence of tumor cells in the samples, gene profiles were generated from FNAB specimens by using a commercially available genome-wide microarray platform. To keep the molecular profiles clinically relevant, we asked whether there is a molecular basis for the clinical practice of lumping Her2+ and TN breast cancers together into the same high-risk group. We analysed the molecular phenotype of Her2+/TN breast cancers and developed a novel gene signature, termed the “ClinicoMolecular Triad Classification” (CMTC), which divides all breast cancers into three groups similar to the three risk groups that oncologists refer to. Each CMTC group displayed a unique pattern of oncogenic signaling pathway activities. To determine the clinical significance of the CMTC classification scheme, we correlated the three CMTC groups using standard pathology parameters, and the results were reproduced in a large independent validation cohort. Using multivariate analyses, CMTC was the best among 14 published prognostic gene signatures and clinical receptor statuses in predicting breast cancer recurrence and treatment response.

### Materials and Methods

### Patients and Samples

The primary data set consisted of 161 prospectively recruited, consecutive surgical patients with breast tumors. A total of 172 tissue samples were collected at the University Health Network (UHN) and Mount Sinai Hospital (MSH), Toronto, ON, Canada. We excluded samples from five benign tumors, five ductal carcinoma in situ samples and two with a low RNA integrity number (RIN). That left 149 invasive breast cancers used as the training cohort, including 121 FNABs, 10 core biopsies and 18 fresh frozen tissue specimens from the BioBank at UHN (Toronto, ON, Canada). FNABs were obtained by passing a 25-gauge needle into the tumor 10 to 20 times with suction using a 10-ml syringe. The cells were suspended in CytoLyt solution (Cytyc Corp, Marlborough, Mass., USA) with an aliquot (10% vol/vol) sent for cytological analysis by a cytopathologist (SB). All FNAB samples had 80% or more malignant cells to be included in this study. The remaining cells were centrifuged and resuspended in 500 μl of RNA extraction lysis buffer (Qiagen, Valencia, Calif., USA), then snap-frozen to −80° C. for later processing. Core biopsies were taken by our radiologist (SK) at the time of diagnostic procedures. This study was approved by the Research Ethics Boards at our institutions (UHN and MSH). All patients were recruited prospectively and gave their written informed consent to participate in the study. The clinical follow-up data were collected until April 2010 with median follow-up of 31 months. The information for the 149 patients is provided in Table 2.

### RNA Extraction and Microarray Process

After we determined that the tissue samples satisfied cytological criteria, the frozen FNAB lysates were thawed and RNA was extracted using the RNeasy Micro and RNeasy Mini kits (Qiagen) for FNABs and core biopsies and UHN BioBank samples, respectively, according to the manufacturer's protocols. The quality and quantity of the RNA were analyzed using an Agilent 2100 Bioanalyzer (Agilent Technologies, Palo Alto, Calif., USA), and only the samples with a RIN higher than 5.5 were used in this study. The DNA microarray analyses were then performed according to the Illumina Whole-Genome Gene Expression direct hybridization assay protocols (Illumina Inc, San Diego, Calif., USA) at The Centre of Applied Genomics (Toronto, ON, Canada). Briefly, 250 ng of total RNA were reverse-transcribed into cDNA, followed by in vitro transcription amplification to generate biotin-labeled cRNA using the Ambion TotalPrep RNA Amplification Kit (Applied Biosystems/Ambion, Austin, Tex., USA). Next, 750 ng of the labeled cRNA were hybridized to Illumina HumanRef-8 v2 Expression BeadChips (Illumina Inc.) overnight at 58° C. After washing, signals were developed with streptavidin-Cy3, and the BeadChips were scanned with the BeadArray Reader and processed using BeadStudio software obtained from Illumina.

### Microarray Data Sets and Analyses

For the training cohort of 149 breast cancers, scanned Illumina microarray image data were extracted and processed by Gene Expression Module version 3.4 of BeadStudio software (Illumina Inc) using a background subtraction and a quantile normalization method for direct hybridization assays. Normalized hybridization intensity values were adjusted by assigning a constant value of 16 to any intensity value lower than 16, according to the recommendation by the MAQC Consortium [21]. A log₂ expression ratio of an intensity value to the average signal value for each transcript in all samples was calculated. The training cohort microarray data are available at the Gene Expression Omnibus website [GSE:16987] [22].

An independent validation cohort consisting of publicly available gene expression array data from 2,487 breast cancers was compiled from different published original reference data sets that used Agilent and Affymetrix microarray platforms (Table 3). On the basis of the clinical treatment and the end point, four subgroups of the validation cohort were used to validate the CMTC classification derived from the training cohort: (1) 2,239 cancers with follow-up [23-36], (2) 1,058 cancers without adjuvant therapy [24,25-31,34], (3) 756 ER+ cancers with or without ET [24,26-29,33] and (4) 248 breast cancers treated with neoadjuvant chemotherapy and pCR information [37]. The methods of platform-specific data treatment and analyses are described in the methods.

### Methods

Microarray data resources. The primary dataset generated by using Illumina HumanRef-8 v2 Expression BeadChip (http://www.illumina.com/). Total 161 breast tumors were taken between 2006 and 2008 from Princess Margaret Hospital and Mount Sinai Hospital (Toronto, ON) and finally, 149 invasive breast cancers were created as the training cohort (Table 2). The information for the validation microarray datasets [23,24,25,26,27,28,29,30,31,32,33,34,35,36,37,51,] is listed in Table 3. The microarray data and their patient clinical information for the validation dataset with 295 breast cancers from Netherlands Cancer Institute [24,51] were downloaded from websites http://www.rii.com/publications/2002/nejm.html and http://microarray-pubs.stanford.edu/wound_NKI/. The other validation datasets were downloaded from NCBI Gene Expression Omnibus website http://www.ncbi.nlm.nih.gov/geo, using the accession numbers from the respective studies. All microarray data used in this study excluded replicated cases and contained clinical endpoint information. Any type of recurrence, including local recurrence and distant metastasis, was used to analyzed the relapse-free survival. All tumors must come with their clinical ER, PR and Her2 status. If the status is not available from the published materials, a request would be sent to the author, or array expression values of the three genes were used.

### Agilent Microarray Data Processing.

The downloaded Agilent Hu25K data for the 295 breast cancers came with log ratios of the signals for each probe from the tumor relative to pooled sample from all patients [24,51]. The downloaded GEO series matrix files from two Agilent datasets of GSE10886 [23] and GSE6128 [36] were in log 2 ratios of the tumor RNA relative to a modified Stratagene Human Universal Reference RNA, and only arrays in platform GPL1390 were used in the study. To make the two Agilent datasets compatible with other microarray datasets, the log ratios of Agilent Hu25K dataset were converted to log 2 ratios; whereas the log 2 ratios of GSE10886 and GSE6128 datasets was first converted back to ratios and then compared that to the average ratios of all the probes in log 2 format.

### Affymetrix Microarrays Data Processing.

The downloaded Affymetrix CEL data were processed by Expression Console version 1.1.1 of the GeneChip Operating Software (Affymetrix Inc., Santa Clara, Calif.). The Probe Logarithmic Intensity Error Estimation method was used to produce a summary value for each probe set by Quantile normalization and PM-MM protocols. The downloaded GEO series matrix files in normalized intensity values were directly used in next step of data processing. A value of 16 was assigned to any normalized intensity value that was less than 16, according to the recommendation from MAQC Consortium [52]. A log 2 expression ratio of an intensity value to the average signal value for each transcript in all samples was calculated.

### Integration of Published Gene Expression Signatures.

Sixteen gene expression signatures that have previously been reported to have prognostic predictability in breast cancers [23,24,25,26,29,31,38,39,40,41,42,43,45,53,54] are summarized in Table 4. Out of the 16 gene signatures, 14 microarray-based signatures were used to compare and evaluate the gene signature generated in this study. All array probes in the 14 signatures were re-annotated by using the tools in http://www.ncbi.nlm.nih.gov, then their official gene symbols were used to search each array data from every tumor in the training and validation cohorts. All probes that matched to a specific gene symbol were used to classify the tumors. The expression centroid values for each gene in the signatures were used to score the validating data series. The centroid data for PAM50 [23] was available at https://genome.unc.edu/pubsup/breastGEO. If a centroid data was not available in their published materials, −1 was used as the good signature centroid value and +1 for poor signature. A Pearson correlation was calculated to get the quantitative scores of corresponding expression values for the genes in each tumor to the expression centroid values of the genes in each prognostic signature. The classification of Subtype [53], PAM50 [23] and CMTC, the gene signature generated in this study, were based on the nearest expression centroid method [51,53]. The adjusted threshold value of correlation coefficient −0.15 was used for WS [51,43], and 0.4 for 70GS [24,51]. The correlation coefficient value of zero was used as threshold value to classify the validation tumors for other signatures.

### Integration of Published Signaling Pathway Signatures.

Nineteen pathway signatures that enable integration of patterns in predicting activity for oncogenic signaling and other cellular pathways were collected. The training data and methods to develop gene expression signatures for pathway activity have been previously described [28,55]. To test the probabilities of the pathway activity in the 149 breast cancers in training cohort, the predicted activity patterns for the 19 pathways were represented into the three types in CMTC that was generated in this study by using a hierarchical clustering. A Pearson correlation was performed to depict the co-regulation among the pathways.

### Statistics and Data Analysis.

All microarray data were represented as log 2 ratios for the expression analysis of gene transcription and entered into the Acuity software version 4 (Molecular Devices, Sunnyvale, Calif.) with their annotation files and clinical information for data analysis. Variant significance t test and ANOVA test were used to evaluate the differential expression between cancer groups. A Benjamini-Hochberg method was used to control false discovery rate, and the most conservative correction method Bonferroni was applied to the P values of corresponding t tests between different microarray expression patterns. Chi-square test and Fisher's exact test were used to test the significance of the clinical and pathological variables between different cancer types. The hierarchical analysis was used to generate and present the expression patterns. Kaplan-Meier analysis was used to compare patient's survivals in differential gene expression groups, and their differences were determined by the Log-rank Test. Univariate and multivariate analyses of prognostic factors were performed by using Cox proportional hazard method. Receiver Operating Characteristic analysis was used to score the Area Under the Curve. All reported P values were two-sided, and a P value of less than 0.05 was considered statistically significant.

### Illumina Array Quality Measures and Data Processing.

To measure the quality of the Illumina microarray, a control RNA sample was incorporated using Universal Human Reference RNA (Stratagene; La Jolla, Calif.) into each of the 30 Illumina BeadChips. The Reference microarray dataset is available at GEO website with the accession number GSE16984. For each of the 22,184 unique probes in the dataset, there was an average of 42.3±8.1 replicated beads. The correlation analysis of the expression intensity values revealed a very high average correlation coefficient of 0.9908±0077 among the 30 controls. In the sample specimens, the average correlation coefficient was 0.9918±0108 for the 10 pairs of duplicated fine needle aspiration biopsies taken from the same tumors and 0.8491±0407 among different tumors. All duplicates of the cancer samples were combined for each tumor, and a total 149 microarray data of breast cancers was used for next analysis for the selected 149 invasive breast cancers. By adjusting the lowest intensity value, 713 probes with a log 2 ratio value of “0” across all samples were considered as under detectable signals and were eliminated from the next step of the analysis. Respectively, within the 149 breast cancers, the expression levels of ESR1 and ERBB2 from microarray were consistent very well with clinical ER and Her2 status measured by immunohistochemistry or fluorescent in situ hybridization (P<0.0001).

### Generation of Gene Expression Profile for Her2+/TN Phenotype.

Of the 149 breast cancers in the training cohort, 44 were Her2-positive (Her2+) or triple negative (TN, ER−/PR−/Her2−). The 44 Her2+/TN tumors were used as a group to distinguish the gene expression pattern compared to the other 105 tumors. At test was performed to screen the most differentially expressed genes between the two groups. A total of 1428 probes (representing 1376 genes, some genes were represented by multiple oligonucleotide probes in the microarray) were selected at a level of Bonferroni corrected P value less than 0.01. The hierarchical clustering analysis using the 1428-probe set resulted in division of a group of 39 tumors with 36 Her2+/TN status from the other group of 110 tumors with 8 Her2+/TN status. As shown in FIG. 5A, the group with less Her2+/TN tumors can visibly be separated into two subgroups which we labeled as group 1 and 2 according to the gene expression profile, and the group enriched with Her2+/TN tumors was shown as group 3. Because we wanted to look for the molecular basis of dividing breast cancers into 3 groups similar to oncologists in the clinical settings, we went on to perform a second screen using all the differentially expressed genes that were best in separating the 149 breast cancers of the training cohort into three clusters with most Her2+/TN in one group. A total of 1349 probes (1304 genes) were selected at a level of the P value less than 0.001 by an ANOVA test among the three groups. As a result, a more apparent three-cluster pattern was seen using the 1349-probe set (FIG. 5B). Out of the 42 tumors in group 1, only one was Her2+/TN; there are 7 Her2+/TN in the 68 tumors of group 2, and 36 Her2+/TN in the 39 tumors of group 3.

### Results

### Gene Model and Generation of the ClinicoMolecular Triad Classification

Of the 149 evaluable breast cancers in the training cohort (Table 2), all 26 Her2+ tumors and 18 TN tumors were grouped into one group and the remaining 105 into another group in the first round of supervised clustering analysis to identify the differentially expressed genes. After two screens (see Microarray data resources in the Methods section and FIG. 5), a molecular profile of Her2+/TN was obtained with 1,304 genes (1,349 oligonucleotide probes; some genes were represented by multiple oligonucleotide probes in the Illumina BeadChip assay). This molecular profile appeared to divide the 149 tumors into a familiar three-group pattern (FIG. 5B) in which the third group included most of the Her2+/TN tumors. Compared to the 16 published prognostic gene expression signatures (Table 4), a total of 501 genes were found in the list of the 1,304 genes matching 4% to 90.4% of the genes in these prognostic signatures. These overlapped genes included the following: (1) 29% (223 of 769) of the genes in the estrogen-regulated gene expression signature [38] and 14% (10 of 70) of the Rotterdam signature (76GS) [25]; (2) two ER-related gene signatures, 18% (92 of 512) of the intrinsic gene subtype signature (subtype) [6,7] and 56% (28 of 50) of the modified subtype classifier 50-gene prediction analysis of microarray (PAM50) [23]; (3) 10% (106 of 1,025) of the embryonic stem cell-like gene signature [39], 16% (29 of 181) of the “invasiveness” gene signature [40], 20% (32 of 155) of the stroma-derived prognostic predictor [41] and 14% (8 of 58) of the CD44 signature [42]; four stem cell-related gene signatures, 86% (93 of 108) of the Genomic Grade Index (97GS) [26], 90% (75 of 83) of the proliferation gene signature [31], 48% (11 of 23) of the TP53 mutation gene signature [29], 16% (73 of 462) of the wound-response gene signature (WS) [43], 30% of the lethal phenotype gene signature (37GS) [44]; and 42% (26 of 62) of MammaPrint (70GS) [24] and 56% (9 of 16) of Oncotype DX [45], with these latter two being the most widely used gene signatures [19].

To eliminate any potential confounding effects due to these prognostic signatures, we excluded all of the 501 overlapping genes from the list of 1,304 genes and used the remaining 803 genes (828 oligonucleotide probes) to perform a clustering analysis on the 149 tumors. The pattern with three main clusters was again apparent in the dendrogram (FIG. 1A). The differential gene expression patterns were significantly different among the three groups as determined by performing an analysis of variance test (P<0.00001 among the three groups) and a t-test (corrected to P<0.01 between any two groups). We termed this 803-gene signature the “ClinicoMolecular Triad Classification,” in which CMTC-3 contains most of the Her2+/TN tumors (92.3%). This 803-gene set was used as the new CMTC classifier for further analysis to categorize breast cancer in the validation cohort by a correlation method (see Microarray data resources in the Methods Section).

### ClinicoMolecular Triad Classification Correlates to Clinical Parameters of Breast Cancer

To understand the relationship between the gene expression profiles and the clinicopathological characteristics of CMTC, the three CMTC tumor types were compared based on their clinical and pathological parameters in 149 breast cancers in the training cohort and in 2,487 breast cancers in the validation cohort (Table 1). The latter cohort consisted of all evaluable breast cancers from published microarray data that had complete pathological and clinical outcome data. A statistically significant association between CMTC-3 tumors and larger size, high grade, low ER expression and mostly Her2+/TN phenotypes was found in both training and validation cohorts. In contrast, CMTC-1 tumors were smaller and low-grade, had high ER expression and were rarely the Her2+/TN phenotype. CMTC-2 tumors were larger in size and high-grade, had high ER expression and were rarely the Her2+/TN phenotype.

### ClinicoMolecular Triad Classification Displays Unique Patterns in Oncogenic Signaling Pathways

To understand the biological processes underlying our CMTC classification scheme, the three CMTC groups in 149 breast cancers in the training cohort were compared with 19 published microarray-based signaling pathway signatures [18,46] (FIG. 1B). The highest activity was found in oncogenic signaling pathways involving Her2, Myc, E2F1, β-catenin and Ras in CMTC-3 and a negative correlation with the activities of ER, PR and p53 wild-type pathways. In contrast, CMTC-1 tumors demonstrated low activity in Myc, E2F1, β-catenin, Ras, IFN-γ and Her2 signaling pathways and higher activity in ER, PR and p53 wild-type pathways. CMTC-2 was distinct from the other two groups in having high activities in most of the oncogenic pathways that differentiated CMTC-1 from CMTC-3, including the ER, phosphatidylinositol 3-kinase (PI3K), Myc and β-catenin pathways.

ClinicoMolecular Triad Classification Unifies Prognostication from Published Prognostic Gene Signatures

Of the 16 published prognostic gene signatures (Table 4), 14 microarray-based signatures were used as risk classifiers to evaluate the 149 breast cancers in the training cohort. Even when all the overlapping genes from these published prognostic gene signatures were excluded from the CMTC classifier gene set, the tumors classified as carrying a “poor prognosis” according to the published prognostic gene signatures were mostly found in CMTC-3 and CMTC-2 and infrequently in CMTC-1 (FIG. 1A). Comparison of the five molecular subtypes [6,7] revealed that all the normal-like tumors were found in CMTC-1, luminal A tumors were distributed in both CMTC1 and CMTC-2, luminal B tumors were mainly found in CMTC-2 and almost all Her2+ and basal-like subtypes were found CMTC-3. A similar distribution of the five molecular subtypes was also observed when we used a newer intrinsic subtype classifier, PAM50 [23], a 50-gene subtype predictor, with more luminal B tumors grouped into CMTC-2 (FIG. 1A).

ClinicoMolecular Triad Classification Correlates with Clinical Outcomes in Breast Cancer

During our first clinical follow-up (mean follow-up=31 months) for the 149 cancers in the training cohort, five recurrences (5 of 39=12.8%) were found in CMTC-3, four (4 of 65=6.2%) were found in CMTC-2 and only one (1 of 45=2.2%) was found in CMTC-1. However, these results were not statistically significant, owing to a low event rate in a short follow-up period (FIG. 1A and Table 1). In the validation breast cancer cohort with long-term follow-up, a significantly higher recurrence rate was observed: 40.5% in CMTC-2 and 39% in CMTC-3 compared to 18.6% in CMTC-1 (Table 1). The Kaplan-Meier analyses for relapse-free survival showed significant differences between CMTC-1 and CMTC-2 and also between CMTC-1 and CMTC-3 breast cancer patients in 2,239 breast cancers overall (FIG. 2A) and in 1,058 breast cancers in which the patients in the validation cohort did not receive any adjuvant therapy (FIG. 2B). CMTC-2 and CMTC-3 patients had similar poor prognoses (FIGS. 2A and 2B). By using a Cox proportional hazards model (Table 5), we compared CMTC-2 and CMTC-3 to CMTC-1 and found that, on the basis of univariate analysis, the hazard ratio (HR) was the highest among all clinicopathological parameters and prognostic signatures (HR=2.40, 95% confidence interval (95% CI)=1.88 to 3.05; P<0.01). By using multivariate analysis, we again found that CMTC had the highest HR (HR=1.73, 95% CI=1.23 to 2.44; P<0.01) among all clinicopathological parameters (age, nodal status, tumor size, tumor grade and receptor status). Among all the prognostic gene signatures, the HR of CMTC was the highest in univariate analysis (HR=2.40, 95% CI=1.88 to 3.05; P<0.0001) and the second highest in multivariate analysis (HR=1.43, 95% CI=1.00 to 2.04; P<0.05). Prediction of recurrence using CMTC was also better than that using receptor status Her2+/TN (Her2+/TN vs non-Her2+/TN) (FIGS. 2C and 2D). Her2+/TN receptor status had a HR of 1.56 in univariate analysis (95% CI=1.27 to 1.91; P<0.01) and 1.35 in multivariate analysis (95% CI=0.91 to 2.00; P=0.13), suggesting that CMTC was more robust than receptor status alone in predicting survival. Hence, CMTC is an independent, strong predictor of recurrence in breast cancer.

ClinicoMolecular Triad Classification Correlates with the Benefits of Endocrine Therapy

In the validation cohort, from among the group of 756 patients with ER+ breast cancer, 405 received ET (390 patients received tamoxifen and 15 patients received an unspecified hormonal therapy) and the remaining 351 did not receive any adjuvant therapy. These two groups were not matched, as they were not derived from a randomized, controlled trial. To identify the association between CMTC and tumor response to ET, we compared the relapse-free survival rates between the two groups. Interestingly, we did not see any benefit of ET (P=0.7735) when we compared the treated and untreated groups in the entire 756 ER+ breast cancer population (FIG. 3A). However, when we divided the 756 ER+ patients into the three CMTC groups, patients in CMTC-1 group had good clinical outcomes in general (FIG. 3B), particularly in the 115 patients treated with ET compared to the 184 untreated patients (FIG. 3C). In fact, the benefit of ET was observed only in the CMTC-1 patients (FIG. 3C) and not in the CMTC-2 and CMTC-3 patients (FIG. 3D). Hence, in our validation cohort, CMTC appeared to predict a benefit from ET in ER+ breast cancer. The other gene signatures could demonstrate only varying degrees of prognostic significance, but did not predict the benefit of ET in the 756 ER+ breast cancer patients (Table 6). When attempts to stratify the patients into different cancer stages were made, only a limited number of cases in the validation cohort had complete staging information. On the basis of all the data available, we observed only a trend toward better relapse-free survival associated with ET in treated versus untreated ER+, CMTC-1 patients at stage I (n=155; P=0.0967) and at stage II or worse (n=142; P=0.0612) (FIG. 6).

### ClinicoMolecular Triad Classification Predicts Complete Pathological Response to Neoadjuvant Chemotherapy

To determine whether CMTC could predict tumor responses to neoadjuvant chemotherapy, 248 breast cancer patients [37] from the validation cohort who received neoadjuvant chemotherapy were studied to determine the relationship between CMTC groups and complete pCR. The highest pCR rate was found in CMTC-3 breast cancer (42%), with much lower pCR rates in CMTC-1 breast cancer (6%) and CMTC-2 breast cancer (8%). Her2+/TN breast cancer patients had a 37% pCR rate (FIG. 4A). To compare the relative ability of receptor status (FIG. 4B) and gene signature (Table 7) to predict pCR, we calculated the area under the curve (AUC) using receiver operating characteristic (ROC) curve analyses. We found that CMTC-3 tumors had the highest AUC value (0.754) compared to Her2+/TN tumors (0.733), Her2+ tumors (0.604) and TN tumors (0.629) (FIG. 4B). In addition, tumors with a high positive correlation with CMTC-3 were significantly correlated with pCR in 111 Her2+/TN tumors (FIG. 4C) and in all 248 chemotherapy-treated tumors (FIG. 4D). When we compared CMTC to 14 published prognostic gene signatures, the highest AUC values were found in the CMTC-3 group in all 248 cancers (0.811) (95% CI=0.76 to 0.86; P<0.001) and in 111 Her2+/TN tumors (0.718) (95% CI=0.63 to 0.80; P<0.001). CMTC was also better than the five intrinsic subtypes and PAM50, as well as the other gene signatures, in predicting pCR (Table 7). For comparison purposes, we also tabulated the sensitivity, specificity, positive predictive value (PPV), negative predictive value (NPV) and accuracy of CMTC in predicting pCR together with the other gene signatures (Table 8). Again, CMTC remained one of the best predictors among these gene signatures, with a good balance between sensitivity and specificity.

### Discussion

Using the gene signature generated from the training cohort, we identified an expression pattern of 1,304 genes that divided the 149 breast cancers into three distinct groups, in which Her2+/TN breast cancer represented 90.4% of the 39 group 3 tumors (FIG. 5B). Of the 1,304 genes, a total of 501 genes overlapped with 16 published prognostic gene signatures (Table 4), matching 4% to 90.4% of the genes in these gene signatures. The high rate of the overlapped genes across the different published gene signatures suggests strong clinical and biological relevance.

To remove any potential confounding effects of the overlapping genes from these published gene signatures, we excluded all of the 501 genes in these published gene signatures that overlapped with our original 1,304-gene set. As a result, a unique 803-gene set (represented by 828 oligonucleotide probes in the Illumina BeadChip assay) was derived. Using the new probe set, we observed a dendrogram with three main clusters which we have termed the “ClinicoMolecular Triad Classification.” In the CMTC, the gene expression pattern of CMTC-1 is completely opposite that of CMTC-3 and results in a distinct, intermediate CMTC-2 (FIG. 1A). The tumors in CMTC-1 and CMTC-2 were mostly ER+ and rarely Her2+/TN. However, of the 44 Her2+/TN tumors, 36 (81.82%) were found in CMTC-3. When we applied the CMTC to 866 Her2+/TN tumors in the 2,487 validation breast cancers, 652 (75.3%) were assigned to the CMTC-3 group (Table 1). Furthermore, the prognostic predictability of CMTC agreed very well with all 14 prognostic gene signatures that were developed independently using different commercial microarray platforms (Table 4). Using these prognostic gene signatures, we found tumors carrying a poor prognosis (from signatures dichotomized into good vs poor prognosis) mostly in the CMTC-2 and CMTC-3 cohorts (FIG. 1A). There was also a close correlation between the five molecular subtypes [6,7,23].

In both training and validation cohorts, the tumors in CMTC-1 were of smaller size and lower grade than tumors in the CMTC-2 and CMTC-3 groups. In the validation cohort, patients in the CMTC-1 cohort were found to have significantly better clinical outcomes than the patients in the CMTC-2 and CMTC-3 groups as demonstrated in 2,239 breast cancers overall (FIG. 2A), 1,058 non-adjuvant-treated cancers (FIG. 2B) and 756 ER+ cancers (FIG. 3B). CMTC was better at predicting clinical outcomes than receptor status alone (FIGS. 2C and 2D), suggesting that it reflects not only the presence of the receptors but also pathway activity. Furthermore, on the basis of the survival data of 1,058 breast cancer patients from the validation cohort who did not receive adjuvant therapy, CMTC prognosticated clinical outcomes significantly better than other published gene signature predictors (Table 5).

Another potential application of our molecular classification is in the prediction of response to adjuvant ET and neoadjuvant chemotherapy. Because of the limitation of using public microarray databases as our validation cohort, we are not able to conclude that CMTC can predict treatment response [47,48]. We were not able to match the treatment arms according to CMTC groups, as they were not randomized as such. Therefore, our intent in this study was to demonstrate an association between CMTC and tumor response to a specific treatment modality by treating each breast cancer case in our validation cohort as a randomly selected patient. CMTC-1 patients appeared to benefit the most from ET in terms of recurrence-free survival compared to patients with ER+ breast cancer who did not receive ET (FIG. 3C), but the benefits of ET were not significant in CMTC-2 and CMTC-3 patients (FIG. 3D). Using the same validation cohort, we found that CMTC also appeared to be better than other published prognostic gene signatures in predicting responses to ET (Table 6). FIG. 3A shows that the benefit of ET was nullified by the fact that most of the ET-treated breast cancers were classified as CMTC-2 and CMTC-3 (n=290) (FIG. 3D) rather than CMTC-1 (n=115) (FIG. 3C). Conversely, most of the group that received no treatment were classified as CMTC-1. Furthermore, it may be possible that ET-treated patients presented at a later stage of their disease than did those who received no treatment, given that the breast cancers classified as CMTC-2 and CMTC-3 were associated with larger tumor size (see preceding paragraph). However, subgroup analyses failed to reach statistical significance, as many cases in the validation cohort lacked complete staging information. On the basis of all the data available, we did detect a trend toward better relapse-free survival in both stage I (n=155; P=0.0967) and stage II or worse (n=142; P=0.0612) CMTC-1 ER+ET-treated patients (FIG. 6). Therefore, in our validation cohort, there was more ET given to so-called “nonresponders” than to “responders.” This brings up an important point: If we do not have a better way to classify ER+ breast cancer and use ET to treat all ER+ breast cancers equally, we may not achieve the desired clinical benefit. This result will need to be confirmed in a randomized, controlled trial with a larger set of ER+ patients and complete staging information.

With regard to response to neoadjuvant chemotherapy, CMTC-3 tumors demonstrated a higher rate of complete pCR to neoadjuvant chemotherapy than the other two CMTC groups did (FIG. 4A). The ability of CMTC to predict pCR after neoadjuvant chemotherapy is not only superior to receptor status (Her2+, TN and Her2+/TN) (FIG. 4B) but also better than the other independent prognostic gene signatures (Table 7). Several gene signatures have been reported to predict pCR or clinical response to specific types of chemotherapy in relatively few, highly selected patients (see Table 1 in [49]). Interestingly, the NPV, PPV and accuracy of these chemotherapy-specific predictors are all within a range similar to that of CMTC, except that CMTC is applicable to different chemotherapeutic regimens in all breast cancers and is prognostic in addition to its predictive power for pCR.

To examine the biological processes that may be involved in CMTC, oncogenic signaling pathway analyses were performed in the training cohort, which showed that CMTC-3 tumors had the highest activity in Her2 and other oncogenic signaling pathways (Myc, E2F1, β-catenin, Ras and IFN-γ) and the lowest activity in ER, PR and wild-type p53 pathways (FIG. 1B). This oncogenic pathway pattern was completely opposite to that of CMTC-1 tumors. CMTC-2 was distinct from the other two groups in having high activity in most of the oncogenic pathways that differentiated CMTC-1 from CMTC-3. Unlike CMTC-1 and CMTC-3 tumors, CMTC-2 tumors did not respond well to the two common treatment strategies, namely, ET and chemotherapy. To find new molecular targets for CMTC-2 tumors, our next study will focus on the molecular profiles of CMTC-2 tumors to identify novel treatment strategies. For example, most CMTC-2 tumors displayed activity in the PI3K and β-catenin pathways, and patients with these tumors may benefit from targeted therapies that disrupt these pathways and ER blockage.

The microarray data of our training cohort were generated predominantly from FNABs taken from an unselected cohort of clinical patients prior to any surgical or medical interventions. Thus, CMTC could be used to help in making treatment decisions at the point of diagnosis. Since CMTC can predict treatment outcomes better than standard surgical pathological parameters, FNABs taken for CMTC group assignment of breast cancer patients in the future may help clinicians decide which patients will benefit from neoadjuvant chemotherapy. Another advantage of using FNABs in our study was the ability to include smaller tumors, which are becoming more common in the era of screening mammography but are routinely excluded from tissue banking because of size limitations, an issue shared by most reported microarray-based prognostic gene signatures. FNABs appeared to collect malignant epithelial cells selectively, as demonstrated by over 80% of malignant cells found in our FNAB specimens. Our microarray data were also very reproducible in duplicate specimens (R=0.9918) (see Microarray data resources).

The gene profiles used to develop CMTC were derived from a commercially available whole-genome microarray platform that has become more affordable than currently available multigene assays, such as MammaPrint (70GS; Agendia Inc, Irvine, Calif., USA) and Oncotype DX (Genomic Health, Redwood City, Calif., USA), which report only a limited number of genes [24,45] at a high cost [19,50]. Furthermore, the clinical application of CMTC may be extended to other commercial genome-wide microarray platforms, as we have demonstrated the reproducibility of CMTC classification in the validation cohort derived independently from different DNA microarray platforms. Another potential application of using a whole-genome microarray platform is the ability to perform pathway activity analyses to provide insights into the biological processes operating within the breast cancer, and this may help to identify novel treatment strategies.

During the past decade, the focus of research has been on finding a gene signature that is both prognostic and predictive with high accuracy while containing only a small number of genes. However, with better microarray technology available at a lower price, we are able to generate microarray data that is highly reproducible and cheaper than any of the commercially available gene signatures. It is well known that single-gene estimation (for example, ER) of individual pathway activity is not accurate enough to predict treatment outcomes (for example, response to ET). Therefore, we believe that by using a larger number of genes, the test will be less susceptible to variations caused by errors in measuring individual genes and thus will result in a more reliable determination of the activity levels of critical oncogenic pathways involved in prognosis and treatment response. With the current vastly improved computing power and storage capacity, we advocate using genome-wide gene profiles to provide a more comprehensive genomic analysis comprising a portfolio of current gene expression profiles that includes CMTC, complete oncogenic pathway analyses and the potential for future analyses if pathway gene signatures are further refined.

Finally, CMTC will need to be validated by prospective, randomized, clinical studies, which are in our future plans. On the basis of our present study, we can say that CMTC has the potential to guide treatment decisions at the time of diagnosis, such as the consideration of treating CMTC-3 breast cancer with neoadjuvant chemotherapy, CMTC-1 with ET alone and CMTC-2 with a combination of ET and chemotherapy in adjuvant settings. We note that CMTC-2 remains a challenge in terms of finding an effective treatment. Additional targeted therapies are necessary, and our oncogenic pathway analyses may provide some guidance in finding targets for CMTC-2.

### Conclusions

On the basis of the Her2+/TN molecular phenotype, we developed an 803-gene signature, the ClinicoMolecular Triad Classification system, which is a new, clinically useful molecular classification scheme for breast cancer. Similarly to current clinical practice, CMTC divides breast cancer into three distinct groups. Patients assigned to CMTC-1 have a better prognosis and significantly benefit from ET. Patients in categories CMTC-2 and CMTC-3 have worse clinical outcomes than CMTC-1 patients, with CMTC-3 tumors tending to display a higher rate of complete pCR in response to neoadjuvant chemotherapies. On the basis of our validation analyses using all evaluable public microarray data, the benefits of our clinicomolecular grouping include (1) the capacity to determine the patient's CMTC group preoperatively, which is especially important in neoadjuvant settings; (2) a further improvement in the ability to predict clinical outcomes and treatment responses to ET and neoadjuvant chemotherapy over clinical receptor status and currently available gene signatures; (3) a molecular classification system that is more generalizable than other prognostic gene signatures (including ER+, ER−, tumors of any size, node-positive or node-negative breast cancer) and was reproducible in the validation cohort, from which the data were generated using different commercially available microarray platforms; and (4) the potential to identify novel molecular targets for each CMTC breast cancer group, especially for CMTC-2 tumors that do not respond well to either ET or chemotherapy. Once we have validated the CMTC system in prospective clinical trials, we plan to introduce it into the clinic to help physicians guide treatment decision-making.

TABLE 1
Clinical and pathological variables in ClinicoMolecular Triad
Classification of breast cancer in training and validation cohorts

| | Training cohort (n = 149) | | | | Validation cohort (n = 2,487) | | | |
|---|---|---|---|---|---|---|---|---|
| | CMTC-1, n | CMTC-2, n | CMTC-3, n | | CMTC-1, n | CMTC-2, n | CMTC-3, n | |
| Variables | (%) | (%) | (%) | P value | (%) | (%) | (%) | P value |
| Total | 45 (30.2) | 65 (43.6) | 39 (26.2) | | 803 (32.3) | 794 (31.9) | 890 (35.8) | |
| Age | | | | | | | | |
| <50 | 15 (33.3) | 18 (27.7) | 17 (43.6) | 2.51E−01 | 231 (39.1) | 202 (34.9) | 299 (43.6) | 6.30E−03 |
| ≧50 | 30 (66.7) | 47 (72.3) | 22 (56.4) | | 360 (60.9) | 377 (65.1) | 386 (56.4) | |
| Size | | | | | | | | |
| ≦2 cm | 23 (51.1) | 21 (32.3) | 11 (28.2) | 5.62E−02 | 361 (54.7) | 209 (32.5) | 235 (32.4) | 1.05E−20 |
| >2 cm | 22 (48.9) | 44 (67.7) | 28 (71.8) | | 299 (45.3) | 434 (67.5) | 490 (67.6) | |
| LN− | 26 (59.1) | 21 (32.3) | 24 (61.5) | 3.27E−03 | 490 (66.8) | 436 (59.2) | 498 (60.3) | 4.37E−03 |
| LN+ | 18 (40.9) | 44 (67.7) | 15 (38.5) | | 243 (33.2) | 301 (40.8) | 328 (39.7) | |
| Grade | | | | | | | | |
| 1 | 13 (28.9) | 1 (1.5) | 0 (0.0) | 5.55E−13 | 270 (39.4) | 81 (12.2) | 29 (3.9) | 3.47E−130 |
| 2 | 27 (60.0) | 30 (46.2) | 6 (15.4) | | 342 (49.9) | 339 (51.2) | 220 (29.6) | |
| 3 | 5 (11.1) | 34 (52.3) | 33 (84.6) | | 74 (10.8) | 242 (36.6) | 495 (66.5) | |
| ER− | 0 (0.0) | 1 (1.5) | 35 (89.7) | 1.16E−27 | 69 (8.6) | 45 (5.7) | 584 (65.6) | 2.60E−211 |
| ER+ | 45 (100) | 64 (98.5) | 4 (10.3) | | 734 (91.4) | 749 (94.3) | 306 (34.4) | |
| Her2+/TN | | | | | | | | |
| No | 42 (93.3) | 60 (92.3) | 3 (7.7) | 1.87E−22 | 715 (89.0) | 668 (84.1) | 238 (26.7) | 1.45E−197 |
| Yes | 3 (6.7) | 5 (7.7) | 36 (92.3) | | 88 (11.0) | 126 (15.9) | 652 (73.3) | |
| Recurrence | | | | | | | | |
| No | 44 (97.8) | 61 (93.8) | 34 (87.2) | 1.49E−01 | 595 (81.4) | 423 (59.5) | 486 (61.0) | 1.99E−22 |
| Yes | 1 (2.2) | 4 (6.2) | 5 (12.8) | | 136 (18.6) | 288 (40.5) | 311 (39.0) | |

CMTC = ClinicoMolecular Triad Classification;
LN = lymph node status;
ER = estrogen receptor;
TN = triple-negative.

TABLE 2
Patient information and tumor pathological data for the training cohort of 149 breast cancers

| | | | | Tumor Size | Tumor | Positive | | | | | | | | Follow-up | CMTC |
|---|---|---|---|---|---|---|---|---|---|---|---|---|---|---|---|
| PTID | RIN | Age | Tumor Type | (cm) | Grade | nodes | LVI | EIC | ER | PR | Her2 | Triple- | Recurrence | (months) | Type |
| GP001 | 6.2 | 42 | IDC | 1.5 | 2 | 0(15) | (−) | (−) | (+) | (−) | (−) | No | n | 44.43 | 3 |
| GP002 | 8.7 | 56 | IDC/Lobular | 2.2 | 3 | 0(3) | (−) | (−) | (−) | (−) | (−) | Yes | n | 39.47 | 2 |
| GP003 | 7.7 | 40 | IDC | 1.5 | 2 | 0(7) | (−) | (−) | (+) | (+) | (−) | No | n | 32.77 | 1 |
| GP004 | 7.0 | 46 | IDC | 2.6 | 2 | 0(5) | (−) | (−) | (+) | (+) | (−) | No | n | 46.43 | 1 |
| GP006 | 7.3 | 63 | IDC | 1.8 | 1 | 0(3) | (−) | (−) | (+) | (−) | (−) | No | n | 46.00 | 1 |
| GP007 | 8.4 | 47 | IDC | 4 | 3 | 8(18) | (+) | (+) | (−) | (−) | (+) | No | n | 39.30 | 3 |
| GP008 | 8.7 | 48 | IDC | 1.9 | 2 | 2(11) | (−) | (−) | (+) | (+) | (−) | No | n | 46.20 | 2 |
| GP009 | 7.1 | 51 | IDC | 2.7 | 3 | 2(20) | (+) | (−) | (+) | (−) | (−) | No | n | 45.73 | 2 |
| GP010 | 7.2 | 72 | IDC | 3 | 3 | 0(1) | (−) | (−) | (+) | (+) | (−) | No | y | 13.60 | 2 |
| GP011 | 7.2 | 84 | IDC | 2.1 | 1 | 0(1) | (+) | (−) | (+) | (+) | (−) | No | n | 43.00 | 1 |
| GP012 | 7.4 | 72 | IDC | 1.5 | 1 | 0(2) | (−) | (−) | (+) | (+) | (−) | No | n | 43.73 | 1 |
| GP013 | 8.2 | 58 | IDC | 3.5 | 2 | 1(17) | (−) | (−) | (+) | (−) | (−) | No | n | 48.33 | 3 |
| GP014 | 7.6 | 49 | IDC | 3.6 | 2 | 0(4) | (−) | (+) | (+) | (+) | (−) | No | n | 43.73 | 1 |
| GP015 | 8.3 | 43 | IDC | 2.9 | 3 | 1(4) | (+) | (−) | (−) | (−) | (−) | Yes | n | 32.30 | 3 |
| GP016 | 8.1 | 73 | IDC | 2.8 | 3 | 2(20) | (−) | (−) | (+) | (−) | (−) | No | n | 23.63 | 2 |
| GP017 | 7.5 | 31 | IDC | 3.5 | 3 | 7(16) | (+) | (−) | (+) | (−) | (+) | No | n | 43.70 | 3 |
| GP018 | 8.7 | 67 | IDC | 2 | 2 | 1(19) | (+) | (−) | (+) | (+) | (−) | No | n | 43.63 | 2 |
| GP019 | 9.1 | 45 | IDC | 2.8 | 3 | 0(3) | (−) | (−) | (−) | (−) | (−) | Yes | n | 22.77 | 3 |
| GP020 | 9.0 | 46 | IDC | 2.8 | 3 | 0(3) | (−) | (−) | (−) | (−) | (−) | Yes | NA | NA | 3 |
| GP021 | 9.1 | 46 | IDC | 0.8 | 1 | 0(3) | (−) | (+) | (+) | (+) | (−) | No | n | 36.90 | 1 |
| GP022 | 9.0 | 68 | IDC/Papilloma | 1.4 | 2 | 0(2) | (−) | (−) | (+) | (+) | (−) | No | n | 25.30 | 1 |
| GP023 | 8.7 | 51 | IDC | 1.4 | 1 | 0(2) | (−) | (−) | (+) | (+) | (−) | No | n | 35.97 | 1 |
| GP024 | 8.1 | 80 | IDC | 2 | 3 | 0(1) | (−) | (−) | (−) | (−) | (−) | Yes | y | 23.13 | 3 |
| GP025 | 9.4 | 46 | IDC | 2 | 2 | 0(2) | (−) | (+) | (+) | (+) | (+) | No | n | 21.63 | 1 |
| GP026 | 8.3 | 48 | IDC/lobular | 2.1 | 2 | 0(1) | (−) | (−) | (+) | (+) | (−) | No | n | 24.97 | 1 |
| GP027 | 7.8 | 69 | IDC | 3.3 | 1 | 4(23) | (−) | (−) | (+) | (+) | (−) | No | n | 36.33 | 1 |
| GP029 | 6.8 | 45 | IDC/lobular | 4.2 | 3 | 1(25) | (+) | (−) | (+) | (+) | (−) | No | n | 29.90 | 1 |
| GP030 | 7.3 | 52 | IDC | 2.8 | 2 | 0(1) | (+) | (−) | (+) | (−) | (−) | No | n | 38.80 | 2 |
| GP031 | 8.6 | 29 | IDC | 1.9 | 3 | 0(4) | (−) | (−) | (−) | (−) | (−) | Yes | n | 23.30 | 3 |
| GP032 | 6.2 | 44 | IDC | 2.3 | 2 | 1(16) | (+) | (−) | (+) | (−) | (−) | No | n | 38.83 | 2 |
| GP033 | 8.4 | 56 | IDC | 2.5 | 3 | 13(28) | (+) | (−) | (+) | (+) | (−) | No | n | 18.23 | 2 |
| GP034 | 7.2 | 57 | IDC | 1 | 2 | 8(35) | (−) | (−) | (+) | (−) | (−) | No | n | 36.20 | 2 |
| GP035 | 6.5 | 50 | IDC | 3.5 | 2 | NA | (+) | (−) | (+) | (+) | (−) | No | NA | NA | 1 |
| GP036 | 7.3 | 70 | IDC | 3 | 2 | 42(44) | (+) | (−) | (+) | (−) | (−) | No | n | 72.97 | 2 |
| GP037 | 5.8 | 61 | IDC | 2.4 | 2 | 2(18) | (−) | (−) | (+) | (−) | (+) | No | y | 41.37 | 2 |
| GP038 | 7.8 | 63 | IDC | 2.3 | 3 | 0(18) | (−) | (−) | (+) | (−) | (−) | No | n | 61.10 | 1 |
| GP039 | 7.6 | 59 | IDC | 4 | 3 | 1(22) | (−) | (−) | (+) | (−) | (−) | No | y | 26.47 | 2 |
| GP040 | 6.0 | 65 | IDC | 2.7 | 3 | 4(17) | (+) | (−) | (+) | (−) | (+) | No | n | 73.03 | 2 |
| GP041 | 7.6 | 43 | IDC | 1.5 | 3 | 4(13) | (+) | (−) | (+) | (+) | (−) | No | y | 54.17 | 1 |
| GP042 | 7.0 | 69 | IDC | 2.5 | 2 | 7(13) | (−) | (−) | (+) | (−) | (−) | No | n | 70.27 | 1 |
| GP043 | 7.5 | 42 | IDC | 2.9 | 3 | 2(27) | (+) | (−) | (+) | (+) | (−) | No | n | 73.07 | 1 |
| GP044 | 6.6 | 57 | IDC | 4.7 | 3 | 7(15) | (+) | (+) | (−) | (−) | (+) | No | n | 51.00 | 3 |
| GP045 | 7.5 | 46 | IDC | 2.2 | 3 | 2(17) | (+) | (+) | (−) | (−) | (+) | No | n | 61.23 | 3 |
| GP046 | 8.4 | 65 | IDC | 1.5 | 2 | 1(2) | (+) | (−) | (+) | (+) | (−) | No | n | 57.37 | 1 |
| GP047 | 8.9 | 35 | IDC | 6 | 2 | 1(18) | (+) | (+) | (+) | (−) | (−) | No | n | 58.83 | 2 |
| GP048 | 8.2 | 73 | IDC | 6 | 1 | 0(9) | (−) | (−) | (+) | (+) | (−) | No | n | 57.37 | 1 |
| GP049 | 7.9 | 44 | IDC | 2.65 | 3 | 0(3) | (−) | (−) | (−) | (−) | (−) | Yes | n | 50.67 | 3 |
| GP050 | 7.0 | 57 | IDC | 1.3 | 3 | 2(14) | (+) | (−) | (+) | (−) | (−) | No | n | 66.27 | 2 |
| GP051 | 7.3 | 71 | IDC | 5 | 2 | 1(11) | (+) | (−) | (+) | (+) | (−) | No | n | 31.83 | 1 |
| GP052 | 6.9 | 54 | IDC | 3.9 | 3 | 1(17) | (+) | (−) | (−) | (−) | (+) | No | y | 41.87 | 3 |
| GP053 | 6.6 | 47 | IDC/Lobular | 6 | 2 | 1(2) | (−) | (−) | (+) | (+) | (−) | No | n | 42.10 | 1 |
| GP054 | 7.9 | 54 | IDC | 2.5 | 2 | 1(22) | (+) | (−) | (+) | (+) | (−) | No | n | 26.73 | 2 |
| GP055 | 9.4 | 69 | IDC | 2.9 | 2 | 1(16) | (+) | (−) | (+) | (+) | (−) | No | n | 36.03 | 1 |
| GP056 | 7.5 | 45 | IDC | 1.7 | 3 | 0(2) | (+) | (−) | (−) | (−) | (+) | No | n | 35.47 | 3 |
| GP057 | 7 | 49 | ILC | 15 | 2 | 5(15) | (−) | (−) | (+) | (+) | (−) | No | n | 36.67 | 1 |
| GP058 | 8.3 | 59 | IDC | 1.6 | 1 | 1(17) | (+) | (−) | (+) | (+) | (−) | No | n | 34.83 | 1 |
| GP059 | 8.3 | 76 | IDC | 2.5 | 2 | 0(1) | (+) | (−) | (+) | (−) | (−) | No | n | 39.90 | 2 |
| GP060 | 7 | 53 | IDC | 2.2 | 3 | 0(6) | (−) | (−) | (−) | (−) | (−) | Yes | n | 41.03 | 3 |
| GP061 | 7.4 | 46 | IDC | 2.4 | 2 | 0(4) | (−) | (+) | (+) | (+) | (−) | No | n | 36.50 | 1 |
| GP062 | 7.1 | 73 | IDC | 1.7 | 2 | 0(2) | (−) | (−) | (+) | (+) | (−) | No | n | 36.97 | 1 |
| GP063 | 7.5 | 67 | IDC | 4 | 3 | 3(30) | (−) | (−) | (+) | (−) | (−) | No | n | 22.63 | 2 |
| GP064 | 6.8 | 45 | IDC | 0.9 | 2 | 0(5) | (+) | (+) | (+) | (+) | (−) | No | n | 35.03 | 1 |
| GP065 | 6.9 | 62 | IDC | 1.9 | 3 | 0(1) | (−) | (−) | (−) | (−) | (−) | Yes | n | 38.30 | 3 |
| GP066 | 8.1 | 73 | IDC | 1.5 | 1 | 1(5) | (−) | (−) | (+) | (+) | (−) | No | n | 37.67 | 1 |
| GP067 | 8.8 | 51 | IDC | 2.2 | 3 | 1(17) | (−) | (+) | (+) | (+) | (−) | No | n | 37.90 | 2 |
| GP068 | 6.5 | 72 | IDC | 1.5 | 2 | 1(13) | (−) | (−) | (+) | (+) | (−) | No | n | 32.13 | 1 |
| GP069 | 7.5 | 58 | ILC | 8.8 | 2 | 5(49) | (−) | (−) | (+) | (+) | (−) | No | n | 33.40 | 2 |
| GP070 | 9.2 | 41 | IDC | 1.4 | 2 | 1(14) | (−) | (−) | (+) | (−) | (−) | No | n | 28.40 | 2 |
| GP071 | 7.1 | 55 | ILC | 16.1 | 2 | 0(23) | (−) | (−) | (+) | (−) | (−) | No | n | 28.50 | 1 |
| GP072 | 8.5 | 40 | IDC | 2 | 2 | 3(17) | (+) | (+) | (+) | (+) | (−) | No | n | 26.37 | 2 |
| GP073 | 8.8 | 60 | IDC | 1.3 | 2 | 1(23) | (−) | (−) | (+) | (+) | (−) | No | n | 24.67 | 2 |
| GP074 | 9 | 32 | IDC | 2.6 | 3 | 1(13) | (+) | (−) | (+) | (−) | (−) | No | n | 37.00 | 2 |
| GP075 | 8.4 | 65 | IDC | 1.8 | 2 | 1(17) | (+) | (−) | (+) | (+) | (−) | No | n | 37.20 | 1 |
| GP076 | 8.8 | 46 | ILC | 2.3 | 2 | 1(21) | (−) | (−) | (+) | (+) | (−) | No | n | 32.73 | 1 |
| GP077 | 8.8 | 52 | IDC | 2 | 3 | 0(2) | (−) | (+) | (+) | (−) | (−) | No | n | 36.07 | 2 |
| GP078 | 7.9 | 58 | IDC | 3 | 3 | 2(18) | (−) | (−) | (+) | (+) | (−) | No | n | 1.80 | 3 |
| GP079 | 7.4 | 58 | IDC | 0.8 | 1 | 0(1) | (−) | (−) | (+) | (+) | (−) | No | n | 26.00 | 1 |
| GP080 | 8.7 | 58 | IDC | 0.3 | 2 | 1(5) | (−) | (−) | (+) | (−) | (−) | No | n | 25.70 | 2 |
| GP082 | 7.3 | 36 | IDC | 3.4 | 3 | 0(3) | (−) | (−) | (−) | (−) | (−) | Yes | n | 32.40 | 3 |
| GP083 | 8.6 | 76 | IDC | 2.7 | 3 | 2(18) | (+) | (−) | (+) | (+) | (−) | No | n | 36.20 | 2 |
| GP084 | 8.5 | 51 | IDC | 2.7 | 3 | 1(11) | (−) | (−) | (+) | (+) | (−) | No | n | 1.43 | 2 |
| GP085 | 9.4 | 47 | IDC | 2.8 | 3 | 1(2) | (−) | (+) | (+) | (+) | (−) | No | n | 14.90 | 2 |
| GP086 | 9.2 | 60 | IDC | 1.5 | 2 | 0(2) | (+) | (+) | (+) | (−) | (−) | No | n | 23.97 | 2 |
| GP087 | 9.2 | 68 | IDC | 2.4 | 3 | 0(3) | (−) | (−) | (−) | (−) | (+) | No | n | 24.67 | 3 |
| GP088 | 9.2 | 59 | IDC | 2.7 | 3 | 0(1) | (−) | (−) | (−) | (−) | (+) | No | n | 18.63 | 3 |
| GP089 | 6.3 | 71 | IDC | 2.4 | 2 | 0(5) | (−) | (+) | (−) | (−) | (+) | No | n | 33.33 | 3 |
| GP094 | 7.2 | 57 | IDC | 1.5 | 1 | 0(3) | (−) | (−) | (+) | (−) | (−) | No | n | 37.37 | 1 |
| GP096 | 8.6 | 53 | ILC | 0.8 | 2 | 0(5) | (+) | (−) | (+) | (+) | (−) | No | n | 35.63 | 2 |
| GP097 | 9.3 | 35 | IDC | 5.9 | 3 | 6(19) | (+) | (+) | (+) | (+) | (−) | No | n | 15.97 | 2 |
| GP098 | 6.9 | 59 | IDC | 1 | 3 | 0(2) | (−) | (+) | (+) | (−) | (+) | No | n | 36.10 | 1 |
| GP099 | 8.8 | 47 | IDC | 1.9 | 2 | 1(19) | (−) | (−) | (+) | (+) | (−) | No | n | 35.60 | 2 |
| GP100 | 9.0 | 68 | IDC | 1.4 | 2 | 0(3) | (−) | (−) | (−) | (−) | (−) | Yes | n | 33.20 | 3 |
| GP101 | 9.5 | 35 | IDC | 2.6 | 2 | 2(5) | (+) | (−) | (−) | (−) | (+) | No | n | 32.60 | 3 |
| GP102 | 9.2 | 55 | IDC | 2.9 | 3 | 0(3) | (+) | (−) | (−) | (−) | (+) | No | n | 15.80 | 3 |
| GP103 | 9.0 | 75 | IDC | 2.3 | 3 | 1(4) | (−) | (−) | (−) | (−) | (+) | No | n | 34.57 | 3 |
| GP104 | 7.4 | 47 | IDC | 2.5 | 3 | 3(24) | (−) | (−) | (+) | (+) | (−) | No | y | 33.53 | 2 |
| GP105 | 9.3 | 64 | IDC | 3 | 3 | 2(38) | (+) | (+) | (+) | (+) | (−) | No | n | 25.47 | 2 |
| GP106 | 8.1 | 66 | IDC | 2.3 | 2 | 1(19) | (+) | (−) | (+) | (+) | (+) | No | n | 28.93 | 1 |
| GP107 | 6.5 | 63 | IDC | 1.6 | 3 | 0(5) | (−) | (−) | (−) | (−) | (−) | Yes | n | 30.73 | 3 |
| GP109 | 6.7 | 53 | IDC | 3.5 | 3 | 2(19) | (−) | (+) | (−) | (−) | (+) | No | n | 33.70 | 3 |
| GP110 | 9.6 | 61 | IDC | 2.2 | 3 | 0(2) | (−) | (−) | (+) | (+) | (−) | No | n | 30.33 | 2 |
| GP111 | 7.3 | 69 | IDC | 1.3 | 2 | 3(10) | (−) | (−) | (+) | (+) | (−) | No | n | 33.17 | 2 |
| GP112 | 5.6 | 66 | ILC | 2.1 | 2 | 2(18) | (−) | (−) | (+) | (+) | (−) | No | n | 28.50 | 1 |
| GP113 | 7.4 | 50 | IDC/ILC | 2.6 | 3 | 0(3) | (+) | (−) | (−) | (−) | (+) | No | n | 27.10 | 3 |
| GP114 | 9.1 | 62 | IDC | 2.5 | 3 | 2(12) | (+) | (−) | (+) | (+) | (−) | No | n | 27.97 | 2 |
| GP115 | 9.0 | 45 | IDC | 2.2 | 3 | 2(17) | (+) | (+) | (+) | (+) | (+) | No | n | 34.03 | 2 |
| GP116 | 6.4 | 85 | IDC | 1.5 | 2 | 0(2) | (−) | (−) | (+) | (+) | (−) | No | n | 20.40 | 1 |
| GP117 | 8.3 | 38 | IDC | 4.5 | 3 | 2(10) | (−) | (+) | (−) | (−) | (+) | No | n | 27.23 | 3 |
| GP119 | 5.8 | 77 | ILC | 2.4 | 2 | 0(2) | (−) | (−) | (+) | (+) | (−) | No | n | 26.20 | 1 |
| GP121 | 7.2 | 53 | IDC | 2.6 | 3 | 1(15) | (+) | (−) | (+) | (−) | (−) | No | n | 23.20 | 2 |
| GP122 | 6.1 | 34 | IDC | 2.5 | 2 | 0(3) | (−) | (−) | (+) | (−) | (−) | No | n | 20.10 | 2 |
| GP123 | 7.3 | 67 | IDC | 2.5 | 3 | 0(2) | (−) | (−) | (+) | (+) | (−) | No | n | 27.33 | 2 |
| GP124 | 7.9 | 41 | IDC | 1.1 | 3 | 0(2) | (−) | (−) | (−) | (−) | (−) | Yes | n | 29.93 | 3 |
| GP125 | 8.2 | 60 | IDC | 3 | 3 | 0(2) | (−) | (−) | (−) | (−) | (+) | No | y | 17.93 | 3 |
| GP127 | 7.7 | 59 | IDC | 2.8 | 3 | 0(4) | (−) | (−) | (+) | (+) | (−) | No | n | 21.67 | 2 |
| GP128 | 7.8 | 65 | IDC | 2.4 | 3 | 0(4) | (−) | (−) | (+) | (−) | (−) | No | n | 29.47 | 2 |
| GP129 | 8.7 | 73 | IDC | 2 | 2 | 0(1) | (−) | (−) | (+) | (+) | (−) | No | n | 26.53 | 2 |
| GP130 | 6.7 | 50 | IDC | 1.1 | 1 | 0(2) | (−) | (+) | (+) | (−) | (−) | No | n | 31.57 | 1 |
| GP131 | 8.5 | 46 | IDC | 1.8 | 3 | 5(35) | (+) | (−) | (−) | (−) | (−) | Yes | n | 29.47 | 3 |
| GP132 | 9.4 | 65 | IDC | 2.5 | 3 | 2(14) | (+) | (−) | (+) | (+) | (−) | No | n | 11.00 | 2 |
| GP133 | 6.7 | 59 | IDC | 10.8 | 3 | 0(0) | (+) | (−) | (+) | (−) | (−) | No | n | 32.00 | 2 |
| GP134 | 6.8 | 55 | IDC | 3 | 3 | 0(6) | (−) | (−) | (−) | (−) | (−) | Yes | n | 26.40 | 3 |
| GP135 | 5.7 | 61 | IDC | 2 | 2 | 16(24) | (−) | (−) | (+) | (+) | (−) | No | n | 27.57 | 2 |
| GP136 | 8.3 | 48 | IMC | 3.2 | 2 | 0(7) | (−) | (+) | (+) | (+) | (−) | No | n | 32.00 | 2 |
| GP137 | 6.9 | 48 | IDC | 6 | 3 | 12(20) | (+) | (−) | (−) | (−) | (+) | No | y | 28.87 | 3 |
| GP138 | 7.2 | 49 | IDC | 1.6 | 2 | 1(20) | (−) | (−) | (+) | (+) | (−) | No | n | 22.70 | 2 |
| GP139 | 7.8 | 75 | ILC | 7 | 3 | 3(17) | (+) | (−) | (+) | (+) | (−) | No | n | 29.60 | 2 |
| GP140 | 8.8 | 42 | IDC | 2.4 | 2 | 1(15) | (−) | (−) | (+) | (−) | (−) | No | n | 18.37 | 2 |
| GP141 | 8.0 | 52 | IDC | 3 | 3 | 1(3) | (+) | (−) | (+) | (−) | (+) | No | n | 25.87 | 2 |
| GP142 | 7.3 | 54 | IDC | 2.1 | 3 | 0(3) | (−) | (−) | (+) | (+) | (−) | No | n | 25.07 | 2 |
| GP143 | 8.4 | 53 | IDC | 3.4 | 3 | 0(3) | (+) | (−) | (−) | (−) | (+) | No | y | 16.00 | 3 |
| GP144 | 7.5 | 53 | IDC | 3.6 | 3 | 14(21) | (−) | (−) | (−) | (−) | (+) | No | n | 28.97 | 3 |
| GP145 | 7.2 | 48 | IMC | 7.5 | 2 | 14(19) | (−) | (−) | (+) | (+) | (−) | No | n | 29.63 | 2 |
| GP146 | 6.2 | 48 | IDC | 1.7 | 2 | 5(14) | (+) | (−) | (+) | (+) | (−) | No | n | 29.23 | 1 |
| GP147 | 7.3 | 57 | IDC | 1.2 | 3 | 0(2) | (−) | (−) | (+) | (+) | (−) | No | n | 29.47 | 2 |
| GP148 | 7.9 | 51 | IDC | 4 | 3 | 2(21) | (+) | (−) | (+) | (+) | (−) | No | n | 22.83 | 2 |
| GP149 | 8.6 | 30 | IDC | 2.4 | 3 | 2(18) | (+) | (−) | (+) | (−) | (−) | No | n | 30.57 | 2 |
| GP150 | 8.0 | 60 | IDC | 1.6 | 1 | 0(1) | (−) | NA | (+) | (−) | (−) | No | n | 28.83 | 2 |
| GP151 | 7.1 | 67 | IDC | 1.2 | 2 | 0(5) | (+) | (−) | (+) | (−) | (−) | No | n | 18.77 | 1 |
| GP152 | 7.5 | 72 | IDC | 2.1 | 2 | 0(3) | (+) | (−) | (+) | (+) | (−) | No | n | 30.13 | 2 |
| GP153 | 7.8 | 43 | IDC | 2.3 | 2 | 0(2) | (−) | (−) | (−) | (−) | (−) | Yes | n | 26.30 | 3 |
| GP154 | 8.3 | 66 | IDC | 1.9 | 3 | 0(4) | (−) | (−) | (+) | (−) | (−) | No | n | 27.33 | 2 |
| GP155 | 5.6 | 69 | IDC | 1.8 | 3 | 0(1) | (−) | NA | (−) | (−) | (−) | Yes | n | 26.53 | 3 |
| GP156 | 8.7 | 52 | IDC | 2.1 | 1 | 0(2) | (−) | (−) | (+) | (+) | (−) | No | n | 21.47 | 1 |
| GP157 | 7.9 | 45 | IDC | 3.2 | 2 | 4(20) | (+) | (−) | (+) | (−) | (−) | No | n | 26.13 | 2 |
| GP158 | 7.8 | 78 | IDC | 1.4 | 3 | 0(1) | (−) | NA | (−) | (−) | (−) | Yes | n | 25.53 | 3 |
| GP159 | 7.3 | 58 | IDC | 1.4 | 2 | 0(3) | (−) | (−) | (+) | (+) | (−) | No | n | 27.03 | 1 |
| GP160 | 8.3 | 81 | IDC | 1.5 | 2 | 2(17) | (+) | (−) | (+) | (+) | (−) | No | n | 25.47 | 2 |
| GP161 | 8.3 | 73 | IDC | 0.8 | 2 | 0(1) | (+) | (+) | (+) | (+) | (−) | No | n | 22.03 | 2 |

The estrogen receptor (ER), progesterone receptor (PR) and Her2/neu (Her2) status were evaluated by immunohistochemistry or by fluorescence in situ hybridization using standard clinical protocols.

TABLE 3
Microarray dataset resource

| | GEO | | | | | | |
|---|---|---|---|---|---|---|---|
| | accessions* | Tumor No. | Used No. | Contained | | | |
| | or other | in the | in the | adjuvant | Clinical | | |
| Data cohorts | availability | dataset | study^{†} | treatment | endpoint | Microarray platform | Reference |
| Training cohort | GSE16987 | 161 | 149 | No | RFS | Illumina HumanRef-8 V2 | This study |
| Validation cohort | See URL | 295 | 295 | Yes | DMFS | Agilent Hu25K | 1, 2 |
| | links# | | | | | | |
| | GSE1456 | 159 | 159 | Yes | RFS | AffymetrixU133 A and B | 3 |
| | GSE2034 | 286 | 286 | No | RFS | Affymetrix U133 A | 4 |
| | GSE2990 | 414 | 380 | Yes | RFS | AffymetrixU133 A and B | 5, 6, 7 |
| | GSE6532 | | | | | | |
| | GSE3494 | 251 | 240 | Yes | RFS | Affymetrix U133 A and B | 8, 9 |
| | GSE4922 | | | | | | |
| | GSE7390 | 198 | 119 | No | RFS | AffymetrixU133 A | 10 |
| | GSE9195 | 77 | 77 | Yes | RFS | Affymetrix U133 Plus2 | 11 |
| | GSE10886 | 245 | 245 | Yes | RFS | Agilent H1A UNC custom | 12, 13 |
| | GSE6128 | | | | | (GPL1390) | |
| | GSE11121 | 200 | 186 | No | DMFS | Affymetrix U133 A | 14 |
| | GSE20194 | 278 | 248 | Yes | pCR | AffymetrixU133 A | 15 |
| | (GSE16716) | | | | | | |
| | GSE21653 | 266 | 252 | Yes | DFS | AffymetrixU133 Plus2 | 16 |

*GEO data are available at: http://www.ncbi.nlm.nih.gov/projects/geo/
^{†}Only individual cases with followed-up data in the validation cohort were included.
#http://www.rii.com/publications/2002/nejm.html and http://microarray pubs.stanford.edu/wound_NKI/

TABLE 4
The ClinicoMolecularTriad Classification (CMTC) and published
independent breast cancer gene expression prognostic signatures

| | | | Number | Number | | |
|---|---|---|---|---|---|---|
| Signature | Signature | | of | of known | Overlapped gene | |
| name | definition | Platform | probes | genes | of in preCMTC | Reference |
| preCMTC* | Pre-ClinicoMolecular Triad | Illumina | 1349 | 1304 | 1304 | This Study |
| | Classification Signature | | | | | |
| CMTC* | ClinicoMolecular Triad | Illumina | 828 | 803 | 803 | This Study |
| | Classification | | | | | |
| 37GS | Iethal phenotype genes | Affymetrix | ~ | 37 | 11 | 18 |
| | signature | | | | | |
| 70GS | MammaPrint | Agilent | 70 | 62 | 26 | 1, 2 |
| 76GS | Rotterdam signature | Affymetrix | 76 | 70 | 10 | 4 |
| 97GS | Genomic Grade Index | Affymetrix | 128 | 108 | 93 | 5 |
| CD44 | CD44 gene signature | SAGE | ~ | 58 | 8 | 19 |
| ERGS | Estrogen-regulated genes | Agilent | 822 | 769 | 223 | 20 |
| | expression signature | | | | | |
| ESGS | Embryonic stem cell-like | Affymetix | 1034 | 1025 | 106 | 21 |
| | gene signature | | | | | |
| IGS | Invasiveness gene | Affymetrix | 186 | 181 | 29 | 22 |
| | signature | | | | | |
| Oncotype | Oncotype DX assay | RT-PCR | ~ | 16 | 9 | 23 |
| P53GS | P53 mutation status gene | Affymetrix | 32 | 23 | 11 | 8 |
| | expression signature | | | | | |
| PAM50 | Prediction analysis of | Agilent | ~ | 50 | 28 | 12 |
| | microarray of 50 genes | | | | | |
| Proliferation | Proliferation metagene | Affymetrix | 97 | 83 | 75 | 14 |
| | signature | | | | | |
| SDPP | Stroma-derived prognostic | Agilent | 163 | 155 | 32 | 24 |
| | predictor | | | | | |
| Subtype | Intrinsic genes subtype | cDNA Array | 552 | 512 | 92 | 25 |
| TGFβRII | Type II TGF-βreceptor gene | Affymetrix | 156 | 149 | 6 | 26 |
| | signature | | (Mouse) | (Human) | | |
| WS | Wound-response gene | cDNAArray | 512 | 462 | 73 | 2, 27 |
| | expression signature | | | | | |

*The 803 genes in CMTC were derived from the 1304 genes in preCMTC minus 501 overlapped genes from 16 independent prognostic gene signatures.

TABLE 5
Univariate and multivariate analyses of standard clinicopathology parameters,
14 independent gene signatures and CMTC as prognostic indicators for relapse
among 1058 breast cancer patients without adjuvant therapy in the validation cohort

| | Univariate analyses | | | | Multivariate analyses | | | |
|---|---|---|---|---|---|---|---|---|
| | Hazard | | | | Hazard | | | |
| Variables | Ratio | 95% CI | P value | n* | Ratio | 95% CI | P value | n* |
| Clinic Findings | | | | | | | | |
| Age | 0.72 | 0.55-0.94 | 1.50E−02 | 586 | 0.81 | 0.61-1.06 | 1.20E−01 | 562 |
| LN | 0.63 | 0.43-0.93 | 2.10E−02 | 1052 | 0.79 | 0.53-1.19 | 2.70E−01 | 562 |
| Size | 1.79 | 1.41-2.27 | 1.40E−06 | 772 | 1.49 | 1.13-1.96 | 4.20E−03 | 562 |
| Grade | 2.37 | 1.67-3.37 | 1.50E−06 | 754 | 1.68 | 1.12-5.52 | 1.30E−02 | 562 |
| ER | 1.47 | 1.18-1.83 | 5.20E−04 | 1058 | 0.94 | 0.59-1.50 | 8.00E−01 | 562 |
| Her2 | 0.71 | 0.55-0.90 | 5.70E−03 | 1058 | 0.73 | 0.49-1.07 | 1.10E−01 | 562 |
| TN | 1.43 | 1.10-1.85 | 6.60E−03 | 1058 | 1.18 | 0.67-2.08 | 5.70E−01 | 562 |
| Her2+/TN | 1.56 | 1.27-1.91 | 2.20E−05 | 1058 | 1.35 | 0.91-2.00 | 1.30E−01 | 562 |
| CMTC | 2.4 | 1.88-3.05 | 1.20E−12 | 1058 | 1.73 | 1.23-2.44 | 1.90E−03 | 562 |
| Gene Signatures^{†} | | | | | | | | |
| 37GS | 1.27 | 1.03-1.57 | 2.70E−02 | 1058 | 0.70 | 0.54-0.90 | 6.00E−03 | 1058 |
| 70GS | 1.39 | 1.13-1.71 | 2.20E−03 | 1058 | 1.17 | 0.94-1.45 | 1.60E−01 | 1058 |
| 76GS | 1.96 | 1.60-2.39 | 4.60E−11 | 1058 | 1.35 | 1.06-1.73 | 1.70E−11 | 1058 |
| 97GS | 2.07 | 1.69-2.54 | 1.50E−12 | 1058 | 1.20 | 0.82-1.74 | 3.50E−01 | 1058 |
| ERGS | 1.89 | 1.54-2.32 | 9.70E−10 | 1058 | 1.14 | 0.79-1.64 | 4.90E−01 | 1058 |
| ESGS | 1.88 | 1.53-2.30 | 1.20E−09 | 1058 | 1.11 | 0.84-1.48 | 4.50E−01 | 1058 |
| IGS | 1.99 | 1.57-2.53 | 1.60E−08 | 1058 | 1.23 | 0.88-1.72 | 2.20E−01 | 1058 |
| P53GS | 1.69 | 1.34-2.12 | 6.10E−06 | 1058 | 1.13 | 0.83-1.53 | 4.40E−01 | 1058 |
| PAM50 | 1.66 | 1.35-2.05 | 1.60E−06 | 1058 | 1.10 | 0.82-1.48 | 5.40E−01 | 1058 |
| Proliferation | 1.8 | 1.47-2.19 | 1.10E−08 | 1058 | 1.18 | 0.92-1.50 | 1.90E−08 | 1058 |
| SDPP | 1.8 | 1.47-2.20 | 1.20E−08 | 1058 | 1.11 | 0.83-1.48 | 5.00E−01 | 1058 |
| Subtype | 1.37 | 1.12-1.68 | 2.00E−03 | 1058 | 0.69 | 0.51-0.93 | 1.50E−02 | 1058 |
| TGFβRII | 1.00 | 0.81-1.23 | 1.00E−00 | 1058 | 0.74 | 0.59-0.92 | 7.10E−03 | 1058 |
| WS | 2.24 | 1.61-3.10 | 1.50E−06 | 1058 | 1.45 | 1.00-2.11 | 4.80E−02 | 1058 |
| CMTC | 2.40 | 1.88-3.05 | 1.20E−12 | 1058 | 1.43 | 1.00-2.04 | 4.90E−02 | 1058 |

*The number of cases on which the information of the specific variable is available in the validation cohort.
^{†}Tumors were dichotomized into good and poor prognosis groups based on 14independent prognostic gene signatures and CMTC; for Subtype and PAM50, normal-like and luminal A were placed in good prognosis group, with luminal B, basal like and Her2 status in poor prognosis group; CMTC-1 was in good prognosis group, with CMTC-2 and CMTC-3 in poor group. See Supplemental methods and Table S3 for detailed information on the gene signatures.

TABLE 6
Association between relapse-free survivals and Her2+/TN status, 14 gene signatures and
CMTC in the 756 ER+ breast cancer patients with or without endocrine therapy (ET)

| | Prognosis of classifiers | | | Prognosis of ET in the good | | |
|---|---|---|---|---|---|---|
| | in the 756 patients* | | | prognosis patients | | |
| Prognostic | No. in good | | | No. in endocrine | | |
| classifiers | prognosis | Chi square | P value | therapy | Chi square | P value |
| Her2+/TN | 641 | 8.7800 | 3.00E−03 | 338 | 0.0002 | 9.89E−01 |
| 37GS | 326 | 9.5950 | 2.00E−03 | 166 | 0.8891 | 3.46E−01 |
| 70GS | 141 | 10.5100 | 1.20E−03 | 44 | 0.1554 | 6.93E−01 |
| 76GS | 497 | 21.4900 | 3.54E−06 | 244 | 0.7537 | 3.85E−01 |
| 97GS | 487 | 47.1900 | 6.42E−12 | 228 | 1.0530 | 3.05E−01 |
| ERGS | 433 | 40.3100 | 2.15E−10 | 198 | 1.4550 | 2.28E−01 |
| ESGS | 430 | 18.9400 | 1.35E−05 | 194 | 0.0041 | 9.49E−01 |
| IGS | 283 | 23.1000 | 1.53E−06 | 130 | 0.0449 | 8.32E−01 |
| P53GS | 313 | 26.5700 | 2.54E−07 | 146 | 0.4157 | 5.19E−01 |
| PAM50 | 340 | 29.3400 | 6.05E−08 | 157 | 0.0142 | 9.05E−01 |
| Proliferation | 485 | 19.6900 | 9.09E−06 | 228 | 0.4680 | 4.94E−01 |
| SDPP | 518 | 18.0000 | 2.21E−05 | 242 | 0.0254 | 8.74E−01 |
| Subtype | 433 | 11.9200 | 6.00E−04 | 210 | 0.2257 | 6.35E−01 |
| TGFβRII | 444 | 0.0026 | 9.59E−01 | 212 | 0.0214 | 8.84E−01 |
| WS | 151 | 20.1000 | 7.35E−06 | 49 | 0.4940 | 4.82E−01 |
| CMTC | 299 | 37.5400 | 8.94E−10 | 115 | 5.0780 | 2.42E−02 |

*See Supplemental methods and Table S3 for details on how each tumor is classified into either a good or a poor prognosis group by individual gene signatures. The Chi square and P values were determined by Log-rank Test.

TABLE 7
Receiver operating characteristic analysis of the ability of independent
gene expression signatures to predict pathological complete responses
in breast cancers with neoadjuvant chemotherapy

| | All cancers (n = 248) | | | Her2+/TN cancers (n = 111) | | |
|---|---|---|---|---|---|---|
| Gene Signatures | AUC | 95% CI | P value | AUC | 95% CI | P value |
| 37GS | 0.615 | 0.53-0.70 | 1.19E−02 | 0.574 | 0.47-0.68 | 1.93E−01 |
| 70GS | 0.634 | 0.56-0.71 | 3.54E−03 | 0.597 | 0.49-0.70 | 8.94E−02 |
| 76GS | 0.578 | 0.49-0.66 | 8.81E−02 | 0.546 | 0.43-0.66 | 4.23E−01 |
| 97GS | 0.747 | 0.67-0.82 | 6.79E−08 | 0.633 | 0.53-0.74 | 1.93E−02 |
| ERGS | 0.735 | 0.66-0.81 | 3.01E−07 | 0.619 | 0.51-0.73 | 3.72E−02 |
| ESGS | 0.693 | 0.62-0.77 | 2.45E−05 | 0.642 | 0.54-0.74 | 1.29E−02 |
| IGS | 0.713 | 0.64-0.79 | 3.18E−06 | 0.626 | 0.52-0.73 | 2.72E−02 |
| P53GS | 0.715 | 0.64-0.79 | 2.57E−06 | 0.551 | 0.44-0.66 | 3.72E−01 |
| PAM50-Basal | 0.801 | 0.74-0.86 | 4.83E−11 | 0.666 | 0.56-0.77 | 3.67E−03 |
| PAM50-Her2 | 0.694 | 0.61-0.78 | 2.30E−05 | 0.583 | 0.47-0.70 | 1.46E−01 |
| PAM50-LumA | 0.798 | 0.74-0.86 | 7.58E−11 | 0.657 | 0.56-0.76 | 5.86E−03 |
| PAM50-LumB | 0.715 | 0.64-0.79 | 2.55E−06 | 0.598 | 0.49-0.71 | 8.49E−02 |
| PAM50-Normal | 0.600 | 0.51-0.69 | 2.97E−02 | 0.555 | 0.44-0.67 | 3.34E−01 |
| Proliferation | 0.675 | 0.60-0.75 | 1.29E−04 | 0.588 | 0.48-0.69 | 1.22E−01 |
| SDPP | 0.767 | 0.69-0.84 | 5.53E−09 | 0.622 | 0.51-0.73 | 3.30E−02 |
| Subtype-Basal | 0.775 | 0.71-0.84 | 1.82E−09 | 0.641 | 0.54-0.75 | 1.32E−02 |
| Subtype-Her2 | 0.780 | 0.72-0.84 | 9.66E−10 | 0.640 | 0.54-0.74 | 1.40E−02 |
| Subtype-LumA | 0.795 | 0.73-0.86 | 1.11E−10 | 0.666 | 0.56-0.77 | 3.56E−03 |
| Subtype-LumB | 0.675 | 0.60-0.75 | 1.31E−04 | 0.630 | 0.52-0.74 | 2.27E−02 |
| Subtype-Normal | 0.530 | 0.44-0.62 | 5.09E−01 | 0.554 | 0.34-0.55 | 3.47E−01 |
| TGFβIIR | 0.548 | 0.46-0.64 | 2.90E−01 | 0.506 | 0.40-0.62 | 9.22E−01 |
| WS | 0.659 | 0.58-0.74 | 5.19E−04 | 0.580 | 0.47-0.69 | 1.62E−01 |
| CMTC1 | 0.790 | 0.72-0.86 | 2.52E−10 | 0.675 | 0.57-0.78 | 2.21E−03 |
| CMTC2 | 0.756 | 0.68-0.83 | 2.20E−08 | 0.632 | 0.52-0.74 | 2.06E−02 |
| CMTC3 | 0.811 | 0.75-0.88 | 1.08E−11 | 0.718 | 0.62-0.81 | 1.29E−04 |

AUC, Area Under the Curve.
See Supplemental methods and Table S3 for detailed information on the gene signatures.

TABLE 8
The prediction of pathological complete responses (pCR) in 248
breast cancer patients with neoadjuvant chemotherapy by CMTC
and 14 independent prognostic gene expression signatures

| Signatures | Sensitivity | Specificity | PPV | NPV | Acc |
|---|---|---|---|---|---|
| 37GS | 58.0 | 63.6 | 28.7 | 85.7 | 62.5 |
| 70GS | 98.0 | 20.2 | 23.7 | 97.6 | 35.9 |
| 76GS | 46.0 | 68.2 | 26.7 | 83.3 | 63.7 |
| 97GS | 78.0 | 57.1 | 31.5 | 91.1 | 61.3 |
| ERGS | 88.0 | 45.5 | 28.9 | 93.8 | 54.0 |
| ESGS | 74.0 | 62.6 | 33.3 | 90.5 | 64.9 |
| IGS | 96.0 | 30.8 | 25.9 | 96.8 | 44.0 |
| P53GS | 94.0 | 28.8 | 25.0 | 95.0 | 41.9 |
| PAM50 | 82.0 | 68.7 | 39.8 | 93.8 | 71.4 |
| Proliferation | 56.0 | 67.2 | 30.1 | 85.8 | 64.9 |
| SDPP | 74.0 | 70.7 | 38.9 | 91.5 | 71.4 |
| Subtype | 76.0 | 73.2 | 41.8 | 92.4 | 73.8 |
| TGFβIIR | 48.0 | 58.6 | 22.6 | 81.7 | 56.5 |
| WS | 94.0 | 15.7 | 22.0 | 91.2 | 31.5 |
| CMTC | 78.0 | 72.7 | 41.9 | 92.9 | 73.8 |

The percentages in sensitivity, specificity, positive predictive value (PPV), negative predictive value (NPV) and diagnostic accuracy (Acc). Tumors were dichotomized into good and poor prognosis groups on all two class prognostic gene signatures, and the poor groups were used to predict pCR. For Subtype and PAM50 signatures, basal-like and Her2 status were grouped to predict pCR and compared to normal-like, luminal A and luminal B subtypes; CMTC-3 was used to predict pCR and to compare with CMTC-1 and CMTC-2 groups. See Supplemental methods and Table S3 for detailed information on the gene signatures.

TABLE 9
The 828 probes and CMTC centroids

| | | | | CMTC1 | CMTC2 | CMTC3 |
|---|---|---|---|---|---|---|
| Illumina Probe | | Entrez | | centroid | centroid | centroid |
| ID | RefSeq ID | Gene ID | Gene symbol | value | value | value |
| ILMN_1755321 | NM_015665 | 8086 | AAAS | −0.079778 | 0.139538 | −0.625128 |
| ILMN_1700461 | NM_025267 | 80755 | AARSD1 | 0.085556 | 0.033231 | −0.547179 |
| ILMN_1742051 | NM_001089 | 21 | ABCA3 | −0.088444 | 0.088000 | −1.204103 |
| ILMN_1743371 | NM_032604 | 84696 | ABHD1 | 0.120000 | −0.300462 | −1.597179 |
| ILMN_1794213 | NM_015407 | 25864 | ABHD14A | 0.212000 | −0.099231 | −0.598205 |
| ILMN_1656940 | NM_014945 | 22885 | ABLIM3 | −0.181556 | −0.172000 | −2.626154 |
| ILMN_1738921 | NM_001607 | 30 | ACAA1 | 0.264222 | −0.120769 | −0.484872 |
| ILMN_1795104 | NM_000017 | 35 | ACADS | 0.081111 | −0.348769 | −1.104615 |
| ILMN_1708672 | NM_005891 | 39 | ACAT2 | −0.722667 | −0.203692 | 0.487179 |
| ILMN_1667018 | NM_021804 | 59272 | ACE2 | −1.548667 | −1.504615 | 0.468974 |
| ILMN_1764321 | NM_152331 | 122970 | ACOT4 | 0.167556 | 0.034308 | −1.352821 |
| ILMN_1740265 | NM_181864 | 11332 | ACOT7 | −0.750444 | −0.022462 | 0.241282 |
| ILMN_1658995 | NM_001033583 | 23597 | ACOT9 | −0.455778 | −0.769077 | 0.578718 |
| ILMN_1657153 | NM_005721 | 10096 | ACTR3 | −0.504889 | −0.262615 | 0.451538 |
| ILMN_1725043 | NM_001012969 | 161823 | ADAL | 0.124222 | −0.074615 | −1.013590 |
| ILMN_1742073 | NM_021116 | 107 | ADCY1 | −0.433111 | −0.572000 | −2.691026 |
| ILMN_1654287 | NM_001116 | 115 | ADCY9 | −0.094444 | 0.230923 | −1.376923 |
| ILMN_1759252 | NM_176801 | 118 | ADD1 | 0.126222 | 0.001077 | −0.392821 |
| ILMN_1660332 | NM_001122 | 123 | ADFP | −0.822889 | −0.910154 | 0.494103 |
| ILMN_1702696 | NM_201252 | 246181 | AFAR3 | −0.046222 | −0.066615 | −2.177692 |
| ILMN_1776153 | NM_018046 | 55109 | AGGF1 | −0.113556 | 0.258615 | −0.568205 |
| ILMN_1728787 | NM_176813 | 155465 | AGR3 | 0.051778 | 0.041077 | −6.263846 |
| ILMN_1673529 | NM_153373 | 85007 | AGXT2L2 | 0.228667 | −0.073538 | −0.670000 |
| ILMN_1726703 | NM_001620 | 79026 | AHNAK | 0.241333 | −0.016308 | −0.960769 |
| ILMN_1690676 | NM_012093 | 26289 | AK5 | 0.256000 | −1.987538 | −2.685897 |
| ILMN_1676592 | NM_012067 | 22977 | AKR7A3 | −0.502889 | −0.109077 | −3.782051 |
| ILMN_1747577 | NM_001003945 | 210 | ALAD | 0.205333 | 0.021231 | −0.695128 |
| ILMN_1785284 | NM_005589 | 4329 | ALDH6A1 | 0.134667 | −0.068154 | −0.806667 |
| ILMN_1711886 | NM_005787 | 10195 | ALG3 | −0.556889 | 0.030462 | 0.258974 |
| ILMN_1800958 | NM_001044385 | 65062 | ALS2CR4 | −0.396889 | −0.282615 | 0.410769 |
| ILMN_1665331 | NM_000481 | 275 | AMT | 0.344000 | −0.460154 | −0.854615 |
| ILMN_1766560 | NM_020690 | 404734 | ANKHD1-EIF4EBP3 | 0.253778 | −0.124000 | −0.716154 |
| ILMN_1716790 | NM_173075 | 323 | APBB2 | −0.074444 | 0.097846 | −1.810256 |
| ILMN_1740772 | NM_133172 | 10307 | APBB3 | 0.253111 | −0.050000 | −0.819231 |
| ILMN_1728471 | NM_006421 | 10565 | ARFGEF1 | −0.356222 | 0.241385 | −0.364615 |
| ILMN_1810712 | NM_015313 | 23365 | ARHGEF12 | 0.287778 | −0.254462 | −0.841026 |
| ILMN_1676626 | NM_002892 | 5926 | ARID4A | 0.139111 | −0.004923 | −0.584103 |
| ILMN_1813091 | NM_001177 | 400 | ARL1 | 0.089556 | 0.161385 | −0.733590 |
| ILMN_1800844 | NM_030978 | 81873 | ARPC5L | −0.282444 | −0.067538 | 0.277179 |
| ILMN_1720604 | NM_014960 | 22901 | ARSG | 0.249333 | 0.056923 | −1.868718 |
| ILMN_1654385 | NM_024701 | 79754 | ASB13 | 0.120222 | 0.065538 | −1.381538 |
| ILMN_1695454 | NM_212556 | 401036 | ASB18 | −0.892444 | 0.377385 | −1.161538 |
| ILMN_1783675 | NM_024095 | 140461 | ASB8 | −0.028222 | 0.209846 | −0.579231 |
| ILMN_1745772 | NM_006828 | 10973 | ASCC3 | −0.490667 | −0.230615 | 0.420513 |
| ILMN_1695414 | NM_018154 | 55723 | ASF1B | −1.304222 | 0.063538 | 0.432051 |
| ILMN_1708778 | NM_000050 | 445 | ASS1 | −1.654444 | −1.729538 | 0.903590 |
| ILMN_1716384 | NM_022374 | 64225 | ATL2 | −0.672889 | −0.511077 | 0.585641 |
| ILMN_1661428 | NM_173694 | 286410 | ATP11C | −0.865333 | −0.826923 | 0.838462 |
| ILMN_1804137 | NM_173694 | 286410 | ATP11C | −0.679556 | −0.521231 | 0.606923 |
| ILMN_1815666 | NM_170665 | 488 | ATP2A2 | −0.409333 | 0.023692 | 0.141795 |
| ILMN_1703046 | NM_005176 | 517 | ATP5G2 | 0.068000 | 0.140154 | −0.432308 |
| ILMN_1721741 | NM_018066 | 54707 | ATPBD1B | 0.132889 | −0.100462 | −0.519744 |
| ILMN_1743829 | NM_002973 | 6311 | ATXN2 | 0.041333 | 0.073231 | −0.385897 |
| ILMN_1782918 | NM_006876 | 11041 | B3GNT1 | 0.035556 | −0.105231 | −0.663333 |
| ILMN_1653749 | NM_004776 | 9334 | B4GALT5 | −0.597333 | −0.314615 | 0.447179 |
| ILMN_1773109 | NM_001703 | 576 | BAI2 | 0.140889 | −0.080308 | −1.280513 |
| ILMN_1734929 | NM_003986 | 8424 | BBOX1 | −1.775111 | −2.964154 | 0.490256 |
| ILMN_1702888 | NM_152618 | 166379 | BBS12 | −0.013333 | 0.130154 | −1.026923 |
| ILMN_1762466 | NM_033028 | 585 | BBS4 | 0.122222 | −0.000308 | −1.136410 |
| ILMN_1695110 | NM_001190 | 587 | BCAT2 | −0.018222 | 0.156462 | −0.833846 |
| ILMN_1698224 | NM_022893 | 53335 | BCL11A | −1.712889 | −2.214615 | 0.642564 |
| ILMN_1744822 | NM_003766 | 8678 | BECN1 | 0.054222 | 0.179077 | −0.732308 |
| ILMN_1692773 | NM_016526 | 51272 | BET1L | 0.133111 | 0.018769 | −0.480513 |
| ILMN_1767549 | NM_001487 | 2647 | BLOC1S1 | −0.068444 | 0.174615 | −0.552308 |
| ILMN_1699989 | NM_138278 | 149428 | BNIPL | 0.130889 | −0.402769 | −1.927179 |
| ILMN_1701711 | NM_183359 | 10902 | BRD8 | 0.076000 | 0.107692 | −0.536410 |
| ILMN_1699728 | NM_000060 | 686 | BTD | 0.356667 | −0.194154 | −0.683077 |
| ILMN_1676221 | NM_001207 | 689 | BTF3 | 0.022000 | 0.125692 | −0.756667 |
| ILMN_1815718 | NM_033637 | 8945 | BTRC | 0.249333 | −0.034462 | −0.904615 |
| ILMN_1772706 | NM_144591 | 119032 | C10orf32 | 0.359556 | −0.071846 | −1.208205 |
| ILMN_1652602 | NM_173573 | 256329 | C11orf35 | 0.213111 | 0.153077 | −1.809487 |
| ILMN_1798270 | NM_020179 | 56935 | C11orf75 | −0.726222 | −1.156154 | 0.761282 |
| ILMN_1777765 | NM_021640 | 60314 | C12orf10 | 0.160667 | 0.090462 | −0.767179 |
| ILMN_1736995 | NM_152440 | 144577 | C12orf66 | −0.469333 | 0.307385 | −0.582564 |
| ILMN_1736816 | NM_145061 | 221150 | C13orf3 | −1.269556 | −0.115077 | 0.631026 |
| ILMN_1777487 | NM_018335 | 55778 | C14orf131 | 0.269556 | −0.090000 | −0.735641 |
| ILMN_1756877 | NM_052873 | 112752 | C14orf179 | 0.181333 | 0.022308 | −0.459487 |
| ILMN_1763091 | NM_194278 | 91748 | C14orf43 | 0.209556 | −0.010000 | −0.440769 |
| ILMN_1806456 | NM_025057 | 80127 | C14orf45 | 0.449778 | −0.179538 | −1.412564 |
| ILMN_1750229 | NM_172365 | 145376 | C14orf50 | 0.160667 | −0.329692 | −1.302821 |
| ILMN_1674662 | NM_152259 | 90381 | C15orf42 | −1.922667 | −0.338000 | 0.745897 |
| ILMN_1765880 | NM_024598 | 79650 | C16orf57 | −0.616889 | −0.388769 | 0.604615 |
| ILMN_1656452 | NM_025108 | 80178 | C16orf59 | −1.168000 | 0.074000 | 0.384359 |
| ILMN_1806149 | NM_206967 | 404550 | C16orf74 | −0.078222 | −0.004615 | −1.744359 |
| ILMN_1790537 | NM_152308 | 116028 | C16orf75 | −1.404889 | −0.083077 | 0.431795 |
| ILMN_1681252 | NM_173621 | 284029 | C17orf44 | 0.567333 | −0.538462 | −1.122051 |
| ILMN_1789643 | NM_017622 | 54785 | C17orf59 | 0.194889 | −0.082923 | −0.495897 |
| ILMN_1713803 | NM_001013672 | 400566 | C17orf97 | 0.211778 | −0.044000 | −1.157692 |
| ILMN_1727540 | NM_018186 | 55732 | C1orf112 | −0.490889 | −0.296615 | 0.514872 |
| ILMN_1787280 | NM_024037 | 79000 | C1orf135 | −1.420889 | −0.171385 | 0.438205 |
| ILMN_1761999 | NM_001004303 | 199920 | C1orf168 | −0.163333 | −0.572615 | −2.124872 |
| ILMN_1682428 | NM_144584 | 113802 | C1orf59 | −0.880444 | −0.826154 | 0.559744 |
| ILMN_1768195 | NM_178840 | 149563 | C1orf64 | −0.073111 | −0.774308 | −4.974615 |
| ILMN_1758806 | NM_004928 | 755 | C21orf2 | 0.238222 | −0.164154 | −0.662821 |
| ILMN_1793572 | NM_153750 | 114035 | C21orf81 | −0.282444 | −0.846308 | −2.618462 |
| ILMN_1684726 | NM_013310 | 29798 | C2orf27 | −0.343778 | −0.070308 | −2.398718 |
| ILMN_1720833 | NM_182626 | 348738 | C2orf48 | −0.910000 | −0.106308 | 0.342308 |
| ILMN_1728581 | NM_016210 | 51161 | C3orf18 | 0.294222 | −0.080462 | −2.089487 |
| ILMN_1795514 | NM_207307 | 90288 | C3orf25 | 0.159333 | −0.091385 | −1.159487 |
| ILMN_1672969 | NM_024616 | 79669 | C3orf52 | −0.382000 | 0.055077 | −1.554615 |
| ILMN_1691557 | NM_199417 | 25915 | C3orf60 | 0.122222 | 0.022923 | −0.649231 |
| ILMN_1758427 | NM_018569 | 55435 | C4orf16 | −0.010000 | 0.118462 | −0.633590 |
| ILMN_1695917 | NM_020199 | 56951 | C5orf15 | 0.237111 | 0.040308 | −0.636154 |
| ILMN_1654609 | NM_053000 | 114915 | C5orf26 | 0.230222 | −0.104923 | −0.721026 |
| ILMN_1677292 | NM_033211 | 90355 | C5orf30 | 0.045778 | 0.192923 | −1.625641 |
| ILMN_1662184 | NM_198566 | 375444 | C5orf34 | −1.089556 | −0.038462 | 0.415641 |
| ILMN_1791650 | NM_173665 | 285600 | C5orf36 | 0.105556 | 0.089385 | −0.723077 |
| ILMN_1756673 | NM_152408 | 134359 | C5orf37 | −0.073111 | 0.217692 | −0.554103 |
| ILMN_1699170 | NM_001017987 | 51149 | C5orf45 | 0.007556 | 0.095077 | −0.912308 |
| ILMN_1673478 | NM_016603 | 51306 | C5orf5 | 0.202667 | −0.023231 | −0.681538 |
| ILMN_1651987 | NM_138493 | 154467 | C6orf129 | −0.728444 | 0.018154 | 0.267436 |
| ILMN_1815039 | NM_033112 | 88745 | C6orf153 | −0.279111 | −0.143231 | 0.300769 |
| ILMN_1666617 | NM_024882 | 79940 | C6orf155 | 0.263333 | −0.405846 | −0.986923 |
| ILMN_1783075 | NM_198468 | 253714 | C6orf167 | −1.164667 | −0.435692 | 0.630000 |
| ILMN_1715096 | NM_032511 | 84553 | C6orf168 | −0.815111 | −0.490308 | 0.617692 |
| ILMN_1772588 | NM_025059 | 80129 | C6orf97 | 0.442222 | −0.119846 | −3.412821 |
| ILMN_1660270 | NM_015622 | 51622 | C7orf28A | −0.546444 | −0.020000 | 0.210256 |
| ILMN_1790315 | NM_001039706 | 79846 | C7orf63 | 0.630444 | −0.423538 | −2.031282 |
| ILMN_1688772 | NM_024035 | 78998 | C8orf51 | −0.668889 | 0.072769 | 0.051282 |
| ILMN_1742074 | NM_032847 | 84933 | C8orf76 | −0.551333 | 0.060154 | 0.164103 |
| ILMN_1681221 | NM_032818 | 84904 | C9orf100 | −1.025556 | −0.108923 | 0.319487 |
| ILMN_1717403 | NM_032818 | 84904 | C9orf100 | −1.098667 | −0.082923 | 0.413333 |
| ILMN_1723709 | NM_144654 | 138162 | C9orf116 | 0.236000 | 0.147077 | −1.594872 |
| ILMN_1686841 | NM_001012502 | 286207 | C9orf117 | 0.399778 | −0.614769 | −3.136923 |
| ILMN_1702197 | NM_178448 | 89958 | C9orf140 | −1.517778 | −0.265385 | 0.586154 |
| ILMN_1673863 | NM_031426 | 83543 | C9orf58 | −1.390889 | −1.149538 | 0.852821 |
| ILMN_1659189 | NM_032310 | 84270 | C9orf89 | 0.007333 | 0.163692 | −0.751026 |
| ILMN_1720998 | NM_001218 | 771 | CA12 | 0.234667 | 0.218000 | −3.956410 |
| ILMN_1762407 | NM_031215 | 81928 | CABLES2 | −0.777556 | 0.032615 | 0.310256 |
| ILMN_1688864 | NM_145200 | 57010 | CABP4 | 0.158222 | −0.776615 | −1.749487 |
| ILMN_1711049 | NM_006030 | 9254 | CACNA2D2 | −0.324667 | −0.047077 | −2.485128 |
| ILMN_1696317 | NM_172364 | 93589 | CACNA2D4 | 0.285556 | −0.347231 | −0.892564 |
| ILMN_1810992 | NM_004341 | 790 | CAD | −0.367111 | −0.270769 | 0.471795 |
| ILMN_1749118 | NM_017422 | 51806 | CALML5 | −3.894000 | −3.697846 | −0.048718 |
| ILMN_1743714 | NM_014550 | 29775 | CARD10 | 0.149556 | −0.060615 | −1.092821 |
| ILMN_1712532 | NM_052813 | 64170 | CARD9 | −0.987111 | −1.250462 | 0.653846 |
| ILMN_1708983 | NM_001082972 | 55259 | CASC1 | 0.430889 | −0.098462 | −2.161795 |
| ILMN_1775935 | NM_177974 | 113201 | CASC4 | 0.010222 | 0.107385 | −0.719744 |
| ILMN_1715437 | NM_144508 | 57082 | CASC5 | −0.607556 | −0.148000 | 0.431282 |
| ILMN_1736568 | NM_032983 | 835 | CASP2 | −0.438222 | −0.050000 | 0.400513 |
| ILMN_1718070 | NM_032996 | 842 | CASP9 | 0.241556 | −0.065385 | −0.457692 |
| ILMN_1813400 | NM_032783 | 84869 | CBR4 | 0.212667 | −0.060154 | −0.727179 |
| ILMN_1770678 | NM_005189 | 84733 | CBX2 | −2.702667 | −1.257077 | 1.211282 |
| ILMN_1657361 | NM_175709 | 23492 | CBX7 | 0.385778 | −0.359077 | −0.871026 |
| ILMN_1682567 | NM_013301 | 29903 | CCDC106 | 0.189333 | 0.148769 | −1.183846 |
| ILMN_1751264 | NM_138771 | 90693 | CCDC126 | −0.133333 | 0.039385 | −1.101795 |
| ILMN_1755707 | NM_206886 | 343099 | CCDC18 | −0.774889 | −0.252923 | 0.452564 |
| ILMN_1718771 | NM_152499 | 149473 | CCDC24 | 0.188000 | 0.094154 | −1.126154 |
| ILMN_1789266 | NM_018246 | 55246 | CCDC25 | 0.248000 | −0.191231 | −0.748974 |
| ILMN_1724487 | NM_052849 | 90416 | CCDC32 | 0.258444 | −0.040308 | −0.649487 |
| ILMN_1683533 | NM_001012506 | 285331 | CCDC66 | 0.193111 | −0.042769 | −0.528718 |
| ILMN_1678086 | NM_138770 | 90557 | CCDC74A | 0.092444 | −0.425385 | −3.494359 |
| ILMN_1728979 | NM_207310 | 91409 | CCDC74B | 0.129333 | −0.426769 | −3.620000 |
| ILMN_1761961 | NM_001031713 | 63933 | CCDC90A | −0.463111 | −0.091077 | 0.270513 |
| ILMN_1799710 | NM_153376 | 257236 | CCDC96 | 0.105556 | 0.144615 | −1.174103 |
| ILMN_1695357 | NM_017785 | 54908 | CCDC99 | −0.646667 | 0.008615 | 0.421026 |
| ILMN_1702247 | NM_037370 | 23582 | CCNDBP1 | 0.309333 | −0.063231 | −0.618974 |
| ILMN_1765717 | NM_001761 | 899 | CCNF | −0.918000 | 0.126308 | 0.223590 |
| ILMN_1813431 | NM_019084 | 54619 | CCNJ | −0.766000 | −0.365385 | 0.554359 |
| ILMN_1722502 | NM_001009186 | 908 | CCT6A | −0.431333 | 0.032769 | 0.184359 |
| ILMN_1659727 | XM_001129302 | 146059 | CDAN1 | 0.122444 | 0.076615 | −0.603077 |
| ILMN_1651942 | NM_212530 | 994 | CDC25B | −1.112667 | −0.321692 | 0.500000 |
| ILMN_1764927 | NM_152243 | 11135 | CDC42EP1 | −0.331556 | −0.736769 | 0.549231 |
| ILMN_1660654 | NM_152562 | 157313 | CDCA2 | −1.603556 | −0.614615 | 0.702821 |
| ILMN_1812557 | NM_176096 | 80279 | CDK5RAP3 | 0.140889 | 0.007692 | −0.692821 |
| ILMN_1751411 | NM_016952 | 50937 | CDON | −0.003111 | −0.234923 | −2.276154 |
| ILMN_1693014 | NM_005194 | 1051 | CEBPB | −0.534889 | −0.475538 | 0.604103 |
| ILMN_1711208 | NM_001408 | 1952 | CELSR2 | 0.137111 | −0.153385 | −1.557692 |
| ILMN_1693221 | NM_022909 | 64946 | CENPH | −1.326222 | 0.134462 | 0.137436 |
| ILMN_1737195 | NM_022145 | 64105 | CENPK | −1.414222 | 0.216462 | 0.098205 |
| ILMN_1742779 | NM_033319 | 91687 | CENPL | −0.936222 | −0.019692 | 0.450000 |
| ILMN_1681008 | NM_006568 | 10668 | CGRRF1 | 0.120444 | 0.056308 | −0.658462 |
| ILMN_1674231 | NM_005441 | 8208 | CHAF1B | −0.914889 | −0.145231 | 0.370513 |
| ILMN_1815124 | NM_016139 | 51142 | CHCHD2 | −0.215333 | 0.099846 | 0.062564 |
| ILMN_1673026 | NM_017812 | 54927 | CHCHD3 | −0.548667 | −0.003692 | 0.231026 |
| ILMN_1797530 | NM_032309 | 84269 | CHCHD5 | 0.194667 | 0.130615 | −1.025897 |
| ILMN_1654583 | NM_001270 | 1105 | CHD1 | −0.154000 | 0.185077 | −0.702821 |
| ILMN_1671893 | NM_014453 | 27243 | CHMP2A | −0.004444 | 0.150154 | −0.485897 |
| ILMN_1771233 | NM_176812 | 128866 | CHMP4B | −0.009556 | 0.158308 | −0.484103 |
| ILMN_1770044 | NM_000745 | 1138 | CHRNA5 | −1.700667 | −0.805846 | 0.516667 |
| ILMN_1735199 | NM_020313 | 57019 | CIAPIN1 | −0.689556 | 0.005538 | 0.300513 |
| ILMN_1674411 | NM_018204 | 26586 | CKAP2 | −0.741333 | −0.023538 | 0.286667 |
| ILMN_1751776 | NM_152515 | 150468 | CKAP2L | −1.513333 | −0.018308 | 0.634872 |
| ILMN_1719256 | NM_001826 | 1163 | CKS1B | −0.796889 | −0.073077 | 0.469487 |
| ILMN_1709634 | NM_138809 | 134147 | CMBL | −0.166000 | 0.223692 | −1.810256 |
| ILMN_1805765 | NM_153610 | 202333 | CMYA5 | 0.069111 | −0.112154 | −1.616154 |
| ILMN_1753498 | NM_001042532 | 80347 | COASY | 0.162444 | 0.084923 | −0.703333 |
| ILMN_1666364 | NM_144576 | 93058 | COQ10A | 0.131556 | −0.080462 | −0.663590 |
| ILMN_1783985 | NM_182476 | 51004 | COQ6 | 0.215111 | −0.074923 | −0.598462 |
| ILMN_1689070 | NM_016138 | 10229 | COQ7 | −0.110444 | 0.280615 | −0.990000 |
| ILMN_1784294 | NM_016352 | 51200 | CPA4 | −2.286222 | −2.281538 | 0.292564 |
| ILMN_1755954 | NM_014912 | 22849 | CPEB3 | 0.335556 | −0.046000 | −1.080256 |
| ILMN_1801703 | NM_006651 | 10815 | CPLX1 | 0.333556 | −0.729538 | −2.334359 |
| ILMN_1795454 | NM_007007 | 11052 | CPSF6 | −0.674889 | 0.247231 | −0.120000 |
| ILMN_1660223 | NM_001310 | 1389 | CREBL2 | 0.131556 | −0.021846 | −0.617949 |
| ILMN_1742350 | NM_001311 | 1396 | CRIP1 | 0.190444 | 0.028462 | −1.633077 |
| ILMN_1794033 | NM_175918 | 285464 | CRIPAK | 0.243556 | −0.180923 | −0.859231 |
| ILMN_1693090 | NM_021151 | 54677 | CROT | 0.149333 | −0.208462 | −1.412051 |
| ILMN_1796180 | NM_021117 | 1408 | CRY2 | 0.305778 | −0.144308 | −0.744103 |
| ILMN_1779515 | NM_015989 | 51380 | CSAD | 0.051778 | 0.055385 | −1.752821 |
| ILMN_1652024 | NM_001031812 | 1456 | CSNK1G3 | 0.027778 | 0.172923 | −0.646410 |
| ILMN_1660806 | NM_001321 | 1466 | CSRP2 | −1.347778 | −1.873692 | 0.758974 |
| ILMN_1683444 | NM_005808 | 10217 | CTDSPL | 0.192222 | −0.144615 | −0.474615 |
| ILMN_1738718 | NM_007022 | 11068 | CYB561D2 | 0.191556 | 0.028462 | −0.779487 |
| ILMN_1670925 | NM_144607 | 124637 | CYB5D1 | 0.401333 | −0.162923 | −0.912821 |
| ILMN_1696254 | NM_144611 | 124936 | CYB5D2 | 0.395556 | −0.148769 | −1.372564 |
| ILMN_1729237 | NM_016243 | 51706 | CYB5R1 | 0.080000 | 0.041538 | −0.656667 |
| ILMN_1718988 | NM_014764 | 9802 | DAZAP2 | 0.078667 | 0.111231 | −0.434359 |
| ILMN_1730612 | NM_018478 | 55861 | DBNDD2 | 0.192000 | 0.138923 | −1.494872 |
| ILMN_1715555 | NM_001352 | 1628 | DBP | 0.094444 | 0.054154 | −1.080513 |
| ILMN_1803485 | NM_001919 | 1632 | DCI | −0.038222 | 0.157385 | −0.543077 |
| ILMN_1741564 | NM_016221 | 51164 | DCTN4 | 0.029111 | 0.125538 | −0.657179 |
| ILMN_1727001 | NM_014829 | 9879 | DDX46 | −0.048444 | 0.177077 | −0.625641 |
| ILMN_1768772 | NM_206918 | 123099 | DEGS2 | 0.178667 | −0.096615 | −3.264872 |
| ILMN_1728073 | NM_020946 | 57706 | DENND1A | −1.158222 | −0.545231 | 0.487949 |
| ILMN_1791593 | NM_144973 | 160518 | DENND5B | 0.149111 | −0.309385 | −1.367436 |
| ILMN_1814600 | NM_018369 | 55789 | DEPDC1B | −0.909333 | −0.031385 | 0.562564 |
| ILMN_1654028 | NM_001360 | 1717 | DHCR7 | −0.962889 | −0.428769 | 0.401026 |
| ILMN_1795822 | NM_133375 | 115752 | DIS3L | 0.109333 | 0.075231 | −0.642821 |
| ILMN_1736704 | NM_001037954 | 85458 | DIXDC1 | 0.476444 | −0.485692 | −0.973333 |
| ILMN_1671257 | NM_001363 | 1736 | DKC1 | −0.433556 | −0.072154 | 0.349744 |
| ILMN_1768595 | NM_001365 | 1742 | DLG4 | 0.294444 | −0.302308 | −1.040256 |
| ILMN_1688505 | NM_201262 | 56521 | DNAJC12 | −0.268667 | −0.235692 | −3.504103 |
| ILMN_1725773 | NM_201262 | 56521 | DNAJC12 | −0.102667 | −0.139846 | −2.454359 |
| ILMN_1803073 | NM_021800 | 56521 | DNAJC12 | −0.275333 | −0.251846 | −3.545128 |
| ILMN_1785177 | NM_032364 | 85406 | DNAJC14 | −0.032222 | 0.182769 | −0.486154 |
| ILMN_1687683 | NM_005528 | 3338 | DNAJC4 | 0.236889 | −0.189846 | −0.660769 |
| ILMN_1799516 | NM_015190 | 23234 | DNAJC9 | −0.433778 | −0.063538 | 0.359487 |
| ILMN_1719616 | NM_005223 | 1773 | DNASE1 | −1.156222 | 0.027385 | −0.383077 |
| ILMN_1679912 | NM_206831 | 285381 | DPH3 | −0.539778 | −0.120154 | 0.344103 |
| ILMN_1658992 | NM_003859 | 8813 | DPM1 | −0.440889 | 0.116769 | 0.053333 |
| ILMN_1715905 | NM_024918 | 79980 | DSN1 | −0.708222 | 0.168154 | 0.172564 |
| ILMN_1680544 | NM_080611 | 128853 | DUSP15 | −0.061556 | −0.431231 | −1.904872 |
| ILMN_1697317 | NM_130897 | 83657 | DYNLRB2 | 0.135556 | −0.116769 | −2.408462 |
| ILMN_1812523 | NM_001033560 | 161582 | DYX1C1 | 0.136222 | −0.133538 | −1.070513 |
| ILMN_1777233 | NM_004091 | 1870 | E2F2 | −1.440889 | −0.259385 | 0.617179 |
| ILMN_1652143 | NM_001949 | 1871 | E2F3 | −0.560667 | −0.383692 | 0.531282 |
| ILMN_1782551 | NM_001951 | 1875 | E2F5 | −0.832444 | −0.147538 | 0.342308 |
| ILMN_1798210 | NM_203394 | 144455 | E2F7 | −1.830222 | −0.090615 | 0.017692 |
| ILMN_1762883 | NM_032331 | 9718 | ECE2 | −1.121778 | −0.169385 | 0.522308 |
| ILMN_1662741 | NM_004720 | 9170 | EDG4 | −0.337556 | −0.317231 | 0.378718 |
| ILMN_1738383 | NM_001961 | 1938 | EEF2 | 0.257556 | −0.174000 | −0.334103 |
| ILMN_1669465 | NM_022785 | 64800 | EFCAB6 | 0.323556 | −0.294462 | −1.293846 |
| ILMN_1655497 | NM_001417 | 1975 | EIF4B | 0.224889 | −0.035385 | −0.500769 |
| ILMN_1772486 | NM_006874 | 1998 | ELF2 | 0.152667 | 0.030154 | −0.511282 |
| ILMN_1716843 | NM_017770 | 54898 | ELOVL2 | −0.614889 | −0.486462 | −3.687436 |
| ILMN_1709132 | NM_018255 | 55250 | ELP2 | −0.024000 | −0.035231 | −1.492308 |
| ILMN_1744068 | NM_018091 | 55140 | ELP3 | 0.167333 | −0.099231 | −0.593590 |
| ILMN_1750102 | NM_152463 | 146956 | EME1 | −1.262889 | −0.035231 | 0.211026 |
| ILMN_1791990 | NM_012155 | 24139 | EML2 | 0.160444 | 0.033538 | −0.956923 |
| ILMN_1718297 | NM_019063 | 27436 | EML4 | −0.257778 | −0.426000 | 0.511282 |
| ILMN_1655536 | NM_020189 | 56943 | ENY2 | −0.586222 | −0.055846 | 0.315128 |
| ILMN_1802646 | NM_004445 | 2051 | EPHB6 | −1.573778 | −2.101692 | 0.765641 |
| ILMN_1707267 | NM_001005915 | 2065 | ERBB3 | 0.011556 | 0.208615 | −1.148205 |
| ILMN_1730622 | NM_016337 | 51466 | EVL | 0.474444 | −0.084615 | −1.476667 |
| ILMN_1651628 | NM_019053 | 54536 | EXOC6 | 0.062222 | 0.130462 | −0.798974 |
| ILMN_1697736 | NM_014285 | 23404 | EXOSC2 | −0.378889 | −0.005692 | 0.233590 |
| ILMN_1745271 | NM_019037 | 54512 | EXOSC4 | −0.552667 | 0.095846 | 0.025128 |
| ILMN_1699018 | NM_198947 | 374393 | FAM111B | −0.934000 | −0.154923 | 0.287179 |
| ILMN_1721089 | NM_014612 | 23196 | FAM120A | 0.227778 | 0.079846 | −0.621282 |
| ILMN_1669203 | NM_198841 | 158293 | FAM120AOS | 0.175556 | 0.060000 | −0.652051 |
| ILMN_1743846 | NM_152424 | 139285 | FAM123B | −0.712667 | −0.440769 | 0.607692 |
| ILMN_1717184 | NM_025029 | 80097 | FAM128B | −0.300222 | 0.047385 | −1.564615 |
| ILMN_1811330 | NM_001034850 | 54463 | FAM134B | −0.350889 | 0.204308 | −2.674103 |
| ILMN_1666449 | NM_178126 | 162427 | FAM134C | 0.048889 | 0.047231 | −0.586667 |
| ILMN_1712577 | NM_198507 | 345757 | FAM174A | 0.037333 | 0.275846 | −1.195641 |
| ILMN_1652797 | NM_207446 | 400451 | FAM174B | 0.305556 | −0.042462 | −1.333846 |
| ILMN_1769092 | NM_018166 | 55194 | FAM176B | 0.169111 | −0.207846 | −1.945385 |
| ILMN_1778876 | NM_015091 | 23116 | FAM179B | 0.110667 | 0.084462 | −0.818974 |
| ILMN_1809400 | NM_016623 | 51571 | FAM49B | −0.877333 | −0.154923 | 0.369744 |
| ILMN_1814924 | NM_145037 | 91775 | FAM55C | −0.319333 | 0.021692 | −1.603333 |
| ILMN_1655498 | NM_031478 | 83723 | FAM57B | −0.785111 | −0.384615 | −2.627179 |
| ILMN_1777322 | NM_144963 | 157769 | FAM91A1 | −1.349556 | −0.104308 | 0.331026 |
| ILMN_1698252 | NM_152633 | 2187 | FANCB | −1.021111 | −0.293692 | 0.594872 |
| ILMN_1683112 | NM_000136 | 2176 | FANCC | −0.623556 | −0.227231 | 0.353590 |
| ILMN_1712122 | NM_033084 | 2177 | FANCD2 | −1.086222 | −0.157231 | 0.585897 |
| ILMN_1810703 | NM_001018115 | 2177 | FANCD2 | −0.955556 | −0.139692 | 0.507949 |
| ILMN_1768717 | NM_021922 | 2178 | FANCE | −0.968444 | −0.317077 | 0.640769 |
| ILMN_1729948 | NM_032228 | 84188 | FAR1 | −0.081333 | 0.079538 | −0.746410 |
| ILMN_1754795 | NM_005245 | 2195 | FAT | −0.574000 | −1.680923 | 0.582308 |
| ILMN_1719452 | NM_024326 | 79176 | FBXL15 | 0.155111 | −0.030462 | −0.406923 |
| ILMN_1673370 | NM_012161 | 26234 | FBXL5 | 0.065778 | 0.069231 | −0.748205 |
| ILMN_1733164 | NM_018693 | 80204 | FBXO11 | −0.916444 | −0.254923 | 0.511538 |
| ILMN_1755281 | NM_152676 | 201456 | FBXO15 | −0.035556 | −0.083077 | −1.645128 |
| ILMN_1754811 | NM_030793 | 81545 | FBXO38 | 0.028000 | 0.132615 | −0.528718 |
| ILMN_1710676 | NM_012177 | 26271 | FBXO5 | −0.901111 | −0.231692 | 0.568718 |
| ILMN_1671427 | NM_022039 | 6468 | FBXW4 | 0.206667 | −0.014769 | −0.486923 |
| ILMN_1772686 | NM_033086 | 89846 | FGD3 | 0.610667 | −0.553692 | −2.347436 |
| ILMN_1654194 | NM_024666 | 79719 | FLJ11506 | −0.027111 | 0.145231 | −0.613590 |
| ILMN_1726930 | NM_024941 | 80006 | FLJ13611 | −0.061333 | 0.216308 | −0.747436 |
| ILMN_1815114 | NM_207477 | 400931 | FLJ27365 | 0.241111 | −0.345385 | −1.132051 |
| ILMN_1717265 | NM_001039212 | 222183 | FLJ37078 | −0.147111 | −0.049846 | −1.114615 |
| ILMN_1666633 | NM_152684 | 202020 | FLJ39653 | 0.020444 | 0.110615 | −1.159744 |
| ILMN_1732143 | NM_207436 | 400077 | FLJ42957 | −0.413778 | −0.455385 | −2.231538 |
| ILMN_1766363 | NM_004119 | 2322 | FLT3 | 0.235111 | −0.679538 | −1.626410 |
| ILMN_1730491 | NM_052905 | 114793 | FMNL2 | −0.910444 | −0.955692 | 0.792051 |
| ILMN_1737343 | NM_001008738 | 96459 | FNIP1 | 0.132889 | 0.127538 | −0.618462 |
| ILMN_1716925 | NM_152597 | 161835 | FSIP1 | −0.878222 | 0.106000 | −5.154872 |
| ILMN_1752728 | NM_000147 | 2517 | FUCA1 | 0.159778 | −0.157692 | −0.951538 |
| ILMN_1748836 | NM_025129 | 80199 | FUZ | 0.211333 | −0.005692 | −1.137949 |
| ILMN_1806962 | NM_138387 | 92579 | G6PC3 | 0.089556 | 0.171385 | −0.940256 |
| ILMN_1756469 | NM_000156 | 2593 | GAMT | 0.228444 | 0.113846 | −2.056410 |
| ILMN_1794595 | NM_000156 | 2593 | GAMT | 0.111778 | 0.126308 | −2.209231 |
| ILMN_1741391 | NM_194301 | 253959 | GARNL1 | 0.137778 | 0.096000 | −0.684872 |
| ILMN_1744567 | NM_174942 | 283431 | GAS2L3 | −0.686222 | −0.091538 | 0.227949 |
| ILMN_1710863 | NM_021167 | 57798 | GATAD1 | −0.010222 | 0.164923 | −0.646154 |
| ILMN_1719870 | NM_207418 | 653573 | GCUD2 | −0.962222 | −0.112462 | 0.401538 |
| ILMN_1748116 | NM_001042479 | 54960 | GEMIN8 | 0.156444 | 0.080000 | −0.790000 |
| ILMN_1725678 | NM_005264 | 2674 | GFRA1 | −0.288889 | −0.143385 | −3.569487 |
| ILMN_1746378 | NM_032484 | 84514 | GHDC | 0.268667 | −0.029231 | −1.186154 |
| ILMN_1694279 | NM_024506 | 79411 | GLB1L | 0.320444 | −0.238923 | −0.670769 |
| ILMN_1685871 | NM_000168 | 2737 | GLI3 | 0.267111 | −0.095231 | −1.564359 |
| ILMN_1709771 | NM_013267 | 27165 | GLS2 | 0.025556 | 0.048462 | −1.455385 |
| ILMN_1713290 | NM_018446 | 55830 | GLT8D1 | 0.215556 | 0.001538 | −0.528205 |
| ILMN_1734452 | NM_145016 | 219970 | GLYATL2 | −3.961111 | −4.357538 | −0.061026 |
| ILMN_1677919 | NM_001002002 | 51292 | GMPR2 | 0.109556 | 0.120308 | −0.518718 |
| ILMN_1691567 | NM_138335 | 132789 | GNPDA2 | 0.078444 | 0.035538 | −0.665641 |
| ILMN_1651642 | NM_152742 | 221914 | GPC2 | −1.414222 | −1.101692 | 0.796667 |
| ILMN_1694106 | NM_015141 | 23171 | GPD1L | 0.253333 | −0.073231 | −0.928462 |
| ILMN_1664723 | NM_024531 | 79581 | GPR172A | −0.565333 | −0.137231 | 0.327179 |
| ILMN_1669317 | NM_018485 | 27202 | GPR77 | 0.144889 | 0.126923 | −0.745897 |
| ILMN_1653263 | NM_052899 | 114787 | GPRIN1 | −1.243778 | −0.409385 | 0.401538 |
| ILMN_1661443 | NM_001012642 | 196996 | GRAMD2 | −0.842000 | −2.171692 | 0.139744 |
| ILMN_1721732 | NM_031415 | 56169 | GSDMC | −2.640000 | −2.651692 | 0.698205 |
| ILMN_1709085 | NM_031965 | 83903 | GSG2 | −0.997111 | −0.070769 | 0.487436 |
| ILMN_1740234 | NM_183239 | 119391 | GSTO2 | 0.346667 | −0.280154 | −1.026923 |
| ILMN_1746171 | NM_004893 | 9555 | H2AFY | −0.308444 | 0.184308 | −0.088974 |
| ILMN_1772731 | NM_005326 | 3029 | HAGH | −0.142889 | 0.262154 | −0.617692 |
| ILMN_1737642 | NM_005333 | 3052 | HCCS | −0.422222 | 0.111077 | 0.085128 |
| ILMN_1724720 | NM_002111 | 3064 | HD | 0.055556 | −0.013692 | −0.728974 |
| ILMN_1684690 | NM_024827 | 79885 | HDAC11 | 0.123556 | 0.109231 | −1.133333 |
| ILMN_1767747 | NM_001527 | 3066 | HDAC2 | −0.571778 | −0.393692 | 0.620769 |
| ILMN_1765621 | NM_004494 | 3068 | HDGF | −0.550444 | −0.120769 | 0.474103 |
| ILMN_1702265 | NM_032124 | 84064 | HDHD2 | 0.181111 | −0.008615 | −0.592308 |
| ILMN_1808219 | NM_182922 | 55027 | HEATR3 | −1.033556 | 0.068769 | 0.152308 |
| ILMN_1694268 | NM_018645 | 55502 | HES6 | −1.210000 | −0.031538 | −0.091026 |
| ILMN_1701006 | NM_144608 | 124790 | HEXIM2 | 0.102667 | 0.148154 | −1.198462 |
| ILMN_1735548 | NM_002114 | 3096 | HIVEP1 | −0.347333 | −0.186769 | 0.407179 |
| ILMN_1654268 | NM_002129 | 3148 | HMGB2 | −0.774444 | 0.072769 | 0.223077 |
| ILMN_1688095 | NM_000191 | 3155 | HMGCL | 0.030667 | −0.086154 | −0.903590 |
| ILMN_1651262 | NM_004499 | 3182 | HNRNPAB | −0.350667 | 0.090769 | 0.151282 |
| ILMN_1696485 | NM_031266 | 3182 | HNRNPAB | −0.516889 | 0.131692 | 0.146923 |
| ILMN_1811579 | NM_004838 | 9454 | HOMER3 | −0.748222 | −0.596000 | 0.574103 |
| ILMN_1730442 | NM_020834 | 57594 | HOMEZ | 0.059111 | 0.107385 | −0.785641 |
| ILMN_1697703 | NM_032756 | 84842 | HPDL | −1.175111 | −0.621846 | 0.722564 |
| ILMN_1808713 | NM_002153 | 3294 | HSD17B2 | −2.085556 | −2.870308 | 0.152821 |
| ILMN_1715324 | NM_014234 | 7923 | HSD17B8 | 0.189556 | 0.024000 | −1.391282 |
| ILMN_1797318 | NM_016299 | 51182 | HSPA14 | −0.473778 | −0.072462 | 0.243590 |
| ILMN_1674236 | NM_001540 | 3315 | HSPB1 | −0.277333 | 0.328462 | −0.724615 |
| ILMN_1662070 | NM_000869 | 3359 | HTR3A | −0.349778 | −0.412923 | 0.478974 |
| ILMN_1703041 | NM_000203 | 3425 | IDUA | 0.252889 | −0.108154 | −1.060000 |
| ILMN_1811636 | NM_018010 | 55081 | IFT57 | −0.078444 | 0.034769 | −0.766667 |
| ILMN_1673488 | NM_004970 | 3483 | IGFALS | 0.060667 | −0.234923 | −1.396154 |
| ILMN_1727142 | NM_001556 | 3551 | IKBKB | 0.215333 | −0.070000 | −0.753846 |
| ILMN_1659960 | NM_172374 | 259307 | IL4I1 | −1.946667 | −1.368308 | 0.470513 |
| ILMN_1745172 | NM_004515 | 3608 | ILF2 | −0.317556 | −0.175846 | 0.431795 |
| ILMN_1696311 | NM_017813 | 54928 | IMPAD1 | −0.615333 | −0.141538 | 0.335641 |
| ILMN_1724666 | NM_176878 | 10207 | INADL | 0.434222 | −0.354000 | −1.066410 |
| ILMN_1652647 | NM_004027 | 3631 | INPP4A | −0.547556 | −0.236000 | 0.473333 |
| ILMN_1705699 | NM_001031715 | 64799 | IQCH | 0.101778 | 0.024154 | −0.510513 |
| ILMN_1736223 | NM_022784 | 64799 | IQCH | 0.181556 | 0.033231 | −0.838974 |
| ILMN_1682616 | NM_178827 | 154865 | IQUB | 0.592667 | 0.077846 | −0.564359 |
| ILMN_1734956 | NM_015649 | 26145 | IRF2BP1 | 0.034222 | 0.070769 | −0.458205 |
| ILMN_1713733 | NM_021999 | 9445 | ITM2B | 0.220667 | −0.151692 | −0.442564 |
| ILMN_1789505 | NM_002222 | 3708 | ITPR1 | 0.202222 | −0.275692 | −1.269487 |
| ILMN_1724207 | NM_002225 | 3712 | IVD | 0.149556 | 0.141846 | −1.341026 |
| ILMN_1769382 | NM_198439 | 143879 | KBTBD3 | 0.496667 | −0.353231 | −0.974359 |
| ILMN_1703110 | NM_080671 | 23704 | KCNE4 | −0.544667 | −1.364462 | −3.960769 |
| ILMN_1673769 | NM_002237 | 3755 | KCNG1 | −1.196444 | −0.850462 | 0.557949 |
| ILMN_1726679 | NM_000525 | 3767 | KCNJ11 | 0.056889 | 0.131385 | −1.311026 |
| ILMN_1701173 | NM_004823 | 9424 | KCNK6 | 0.002889 | 0.105231 | −1.341538 |
| ILMN_1800942 | NM_153331 | 200845 | KCTD6 | 0.000667 | 0.083538 | −1.351026 |
| ILMN_1797191 | NM_014656 | 9674 | KIAA0040 | 0.080889 | −0.067231 | −0.983077 |
| ILMN_1762990 | NM_014773 | 9812 | KIAA0141 | 0.258889 | −0.002923 | −0.723333 |
| ILMN_1795704 | NM_014743 | 9778 | KIAA0232 | 0.097556 | 0.083385 | −0.914872 |
| ILMN_1697597 | NM_014774 | 9813 | KIAA0494 | 0.229556 | −0.025692 | −0.499231 |
| ILMN_1797822 | NM_015187 | 23231 | KIAA0746 | −1.079111 | −1.440308 | 0.975897 |
| ILMN_1732343 | NM_025164 | 23387 | KIAA0999 | 0.616889 | −0.527385 | −0.730256 |
| ILMN_1668619 | NM_020853 | 57613 | KIAA1467 | −0.313778 | 0.185077 | −1.943333 |
| ILMN_1728225 | NM_020890 | 57650 | KIAA1524 | −1.564889 | −0.212769 | 0.650000 |
| ILMN_1788347 | NM_033426 | 85457 | KIAA1737 | 0.270000 | −0.057077 | −0.557692 |
| ILMN_1686562 | NM_015254 | 23303 | KIF13B | 0.598889 | −0.602308 | −1.383590 |
| ILMN_1734476 | NM_004520 | 3796 | KIF2A | −0.513556 | −0.077692 | 0.344359 |
| ILMN_1673207 | XM_001129527 | 8462 | KLF11 | −0.519333 | −0.430462 | 0.446923 |
| ILMN_1775875 | NM_172193 | 122773 | KLHDC1 | 0.414889 | −0.011538 | −1.624103 |
| ILMN_1741204 | NM_014315 | 23588 | KLHDC2 | 0.212667 | 0.001385 | −0.736923 |
| ILMN_1801090 | NM_152349 | 125113 | KRT222P | −0.154444 | −0.654462 | −1.756154 |
| ILMN_1801661 | NM_005556 | 3855 | KRT7 | −0.462000 | −1.689538 | 0.414872 |
| ILMN_1719734 | NM_014398 | 27074 | LAMP3 | −1.487778 | −1.411385 | 0.676410 |
| ILMN_1726108 | NM_181746 | 29956 | LASS2 | −0.045111 | 0.167846 | −0.653590 |
| ILMN_1787376 | NM_147190 | 91012 | LASS5 | 0.194889 | −0.053077 | −0.512564 |
| ILMN_1724240 | NM_002296 | 3930 | LBR | −0.571111 | −0.666308 | 0.586410 |
| ILMN_1667577 | NM_014793 | 9836 | LCMT2 | 0.051556 | 0.101692 | −0.734615 |
| ILMN_1679185 | NM_016269 | 51176 | LEF1 | −0.308000 | −0.805846 | −2.268974 |
| ILMN_1782743 | NM_001024668 | 25875 | LETMD1 | 0.145111 | 0.042615 | −0.624359 |
| ILMN_1736077 | NM_006859 | 11019 | LIAS | 0.045333 | 0.145538 | −0.779744 |
| ILMN_1781504 | NM_006859 | 11019 | LIAS | 0.063556 | 0.118923 | −0.750513 |
| ILMN_1664138 | NM_014988 | 22998 | LIMCH1 | 0.393556 | −1.363846 | −0.597949 |
| ILMN_1699471 | NM_173083 | 286826 | LIN9 | −0.642667 | −0.229692 | 0.441026 |
| ILMN_1703487 | NM_006769 | 8543 | LMO4 | −0.768444 | −1.398462 | 0.595897 |
| ILMN_1769449 | NM_203406 | 153364 | LOC153364 | −0.171111 | 0.218462 | −0.939744 |
| ILMN_1687921 | NM_001005920 | 339123 | LOC339123 | 0.157778 | 0.032923 | −0.568462 |
| ILMN_1690911 | NM_001001436 | 388272 | LOC388272 | −0.699556 | −0.019538 | 0.287949 |
| ILMN_1719826 | NM_001013729 | 441956 | LOC441956 | −0.534889 | −1.380000 | −3.187692 |
| ILMN_1755990 | NM_001017971 | 92270 | LOC92270 | 0.122889 | −0.020769 | −0.905897 |
| ILMN_1751016 | NM_198461 | 164832 | LONRF2 | 0.161556 | −0.261692 | −2.357179 |
| ILMN_1651254 | NM_005578 | 4026 | LPP | 0.265556 | −0.366000 | −0.287949 |
| ILMN_1699808 | NM_153377 | 121227 | LRIG3 | −0.586667 | −1.304154 | 0.657949 |
| ILMN_1670272 | NM_014045 | 26020 | LRP10 | 0.187778 | 0.006615 | −0.540256 |
| ILMN_1652826 | NM_005824 | 10234 | LRRC17 | 0.043556 | −0.725538 | −2.621026 |
| ILMN_1727704 | NM_001004055 | 26231 | LRRC29 | 0.126000 | −0.030462 | −0.848462 |
| ILMN_1768818 | NM_033413 | 90506 | LRRC46 | −0.065556 | 0.132769 | −1.685897 |
| ILMN_1667019 | NM_031294 | 83450 | LRRC48 | 0.426444 | −0.440000 | −1.438462 |
| ILMN_1693762 | NM_031294 | 83450 | LRRC48 | 0.593333 | −0.516462 | −1.982564 |
| ILMN_1776967 | NM_178452 | 123872 | LRRC50 | −0.074444 | −0.401692 | −2.457692 |
| ILMN_1685836 | NM_145309 | 220074 | LRRC51 | −0.022222 | 0.084769 | −1.031282 |
| ILMN_1759772 | NM_198075 | 115399 | LRRC56 | 0.235778 | 0.172769 | −1.722821 |
| ILMN_1705746 | NM_018385 | 55341 | LSG1 | −0.534667 | 0.013538 | 0.225128 |
| ILMN_1733960 | NM_032356 | 84316 | LSMD1 | 0.179556 | −0.059385 | −0.410513 |
| ILMN_1776724 | NM_194317 | 130574 | LYPD6 | 0.355333 | −0.846769 | −2.734615 |
| ILMN_1768510 | NM_015274 | 23324 | MAN2B2 | 0.238667 | −0.031692 | −0.650256 |
| ILMN_1673944 | NM_001003897 | 63905 | MANBAL | 0.079556 | 0.088615 | −0.503077 |
| ILMN_1797189 | NM_006301 | 7786 | MAP3K12 | 0.140000 | 0.091231 | −1.432564 |
| ILMN_1695276 | NM_014268 | 10982 | MAPRE2 | −0.652889 | −0.761692 | 0.522564 |
| ILMN_1807042 | NM_002356 | 4082 | MARCKS | −0.515111 | −0.446000 | 0.430769 |
| ILMN_1746012 | NM_052897 | 114785 | MBD6 | 0.064667 | 0.096615 | −0.618718 |
| ILMN_1760174 | NM_020166 | 56922 | MCCC1 | −0.455111 | −0.391077 | 0.407436 |
| ILMN_1659142 | NM_001012333 | 4192 | MDK | −1.186444 | −0.788923 | 0.420769 |
| ILMN_1662263 | NM_138476 | 145553 | MDP-1 | −0.024667 | 0.264923 | −0.939487 |
| ILMN_1793615 | NM_001014811 | 10873 | ME3 | 0.450222 | −1.251385 | −2.055385 |
| ILMN_1736847 | NM_001001654 | 112950 | MED8 | −0.442000 | −0.067231 | 0.300000 |
| ILMN_1779997 | NM_001009813 | 56917 | MEIS3 | 0.140667 | 0.043538 | −1.581282 |
| ILMN_1712583 | NM_024042 | 79006 | METRN | −0.537333 | 0.161077 | −1.968462 |
| ILMN_1738342 | NM_152636 | 196074 | METT5D1 | 0.009111 | 0.167385 | −0.618974 |
| ILMN_1658989 | NM_032246 | 84206 | MEX3B | −1.038667 | −0.958615 | 0.635128 |
| ILMN_1702065 | NM_032889 | 84975 | MFSD5 | −0.062444 | 0.214308 | −0.547949 |
| ILMN_1769601 | NM_033115 | 93627 | MGC16169 | 0.064000 | 0.060923 | −0.615385 |
| ILMN_1737283 | NM_194324 | 286527 | MGC39900 | −2.411556 | −1.662000 | 0.941026 |
| ILMN_1686750 | NM_012215 | 10724 | MGEA5 | 0.155333 | −0.021385 | −0.450000 |
| ILMN_1743232 | NM_032867 | 84953 | MICALCL | 0.042000 | −0.240769 | −1.555385 |
| ILMN_1710684 | NM_138731 | 145282 | MIPOL1 | 0.126889 | 0.008615 | −1.038974 |
| ILMN_1804988 | NM_022151 | 64112 | MOAP1 | 0.141333 | 0.030769 | −0.762821 |
| ILMN_1788878 | NM_178832 | 118812 | MORN4 | −0.070000 | 0.162462 | −0.759231 |
| ILMN_1679995 | NM_016447 | 51678 | MPP6 | −1.481111 | −1.413692 | 0.700256 |
| ILMN_1721774 | NM_173496 | 143098 | MPP7 | 0.025111 | 0.016615 | −1.176923 |
| ILMN_1776515 | NM_023075 | 65258 | MPPE1 | 0.111111 | −0.032308 | −0.462821 |
| ILMN_1697461 | NM_033296 | 93621 | MRFAP1 | 0.007333 | 0.160769 | −0.451282 |
| ILMN_1689774 | NM_203462 | 114932 | MRFAP1L1 | 0.180444 | 0.045692 | −0.501538 |
| ILMN_1671158 | NM_014078 | 28998 | MRPL13 | −0.530222 | 0.023692 | 0.166154 |
| ILMN_1804479 | NM_014161 | 29074 | MRPL18 | −0.513778 | 0.007692 | 0.262821 |
| ILMN_1713143 | NM_007208 | 11222 | MRPL3 | −0.305556 | −0.022923 | 0.224615 |
| ILMN_1681131 | NM_032112 | 84545 | MRPL43 | 0.087111 | 0.045538 | −0.495641 |
| ILMN_1804851 | NM_015969 | 51373 | MRPS17 | −0.417556 | −0.070923 | 0.275897 |
| ILMN_1807095 | NM_033281 | 92259 | MRPS36 | −0.024000 | 0.200615 | −1.075128 |
| ILMN_1760441 | NM_031902 | 64969 | MRPS5 | −0.247778 | −0.168923 | 0.340769 |
| ILMN_1726189 | NM_032597 | 84689 | MS4A14 | 0.317556 | −0.579385 | −1.125128 |
| ILMN_1719471 | NM_002439 | 4437 | MSH3 | −0.037111 | 0.120308 | −0.514103 |
| ILMN_1670723 | NM_078628 | 10943 | MSL3L1 | −0.579333 | −0.205538 | 0.465897 |
| ILMN_1713156 | NM_078629 | 10943 | MSL3L1 | −0.402222 | −0.642923 | 0.435128 |
| ILMN_1660222 | NM_022045 | 27085 | MTBP | −1.490000 | −0.230308 | 0.352308 |
| ILMN_1782504 | NM_015942 | 51001 | MTERFD1 | −0.737333 | 0.018615 | 0.266154 |
| ILMN_1774028 | NM_014637 | 9650 | MTFR1 | −0.843778 | −0.068615 | 0.241026 |
| ILMN_1772521 | NM_015440 | 25902 | MTHFD1L | −1.088889 | −1.037846 | 0.883333 |
| ILMN_1661778 | NM_004923 | 9633 | MTL5 | −0.272889 | 0.179385 | −1.292051 |
| ILMN_1652521 | NM_015458 | 66036 | MTMR9 | 0.176222 | −0.099231 | −0.649231 |
| ILMN_1679071 | NM_001010891 | 345778 | MTX3 | 0.109333 | 0.132923 | −0.619744 |
| ILMN_1756541 | NM_006454 | 10608 | MXD4 | 0.397111 | −0.289538 | −0.496923 |
| ILMN_1746948 | NM_002477 | 4636 | MYL5 | 0.224667 | −0.099846 | −1.087436 |
| ILMN_1774350 | NM_133371 | 91977 | MYOZ3 | −0.502444 | −0.713077 | −2.264103 |
| ILMN_1698441 | NM_012330 | 23522 | MYST4 | −0.044667 | −0.018154 | −1.377436 |
| ILMN_1749838 | NM_198055 | 7593 | MZF1 | 0.368444 | −0.057231 | −0.940769 |
| ILMN_1689665 | NM_001018160 | 8883 | NAE1 | −0.420222 | −0.054615 | 0.282564 |
| ILMN_1653871 | NM_005746 | 10135 | NAMPT | −0.608667 | −0.702462 | 0.379487 |
| ILMN_1705346 | NM_015678 | 26960 | NBEA | −0.006889 | −0.180923 | −1.826923 |
| ILMN_1724718 | NM_003581 | 8440 | NCK2 | −0.421333 | −0.510923 | 0.560769 |
| ILMN_1687768 | NM_181782 | 135112 | NCOA7 | −1.006889 | −1.256769 | 0.877949 |
| ILMN_1751452 | NM_030571 | 80762 | NDFIP1 | 0.108444 | 0.061692 | −0.735128 |
| ILMN_1809931 | NM_006096 | 10397 | NDRG1 | −1.302222 | −0.810923 | 0.471538 |
| ILMN_1767123 | NM_002488 | 4695 | NDUFA2 | −0.015556 | 0.183692 | −0.546667 |
| ILMN_1749738 | NM_031231 | 63941 | NECAB3 | −0.241111 | 0.269538 | −1.026667 |
| ILMN_1733627 | NM_015277 | 23327 | NEDD4L | 0.037778 | −0.078462 | −1.293590 |
| ILMN_1757697 | NM_018248 | 55247 | NEIL3 | −1.475111 | −0.002769 | 0.550256 |
| ILMN_1800445 | NM_001031741 | 152110 | NEK10 | 0.434222 | −0.866308 | −2.443077 |
| ILMN_1778991 | NM_005596 | 4781 | NFIB | −0.897778 | −1.500615 | 0.503077 |
| ILMN_1675130 | NM_005597 | 4782 | NFIC | 0.215778 | −0.217231 | −0.547179 |
| ILMN_1707312 | NM_005384 | 4783 | NFIL3 | −0.838889 | −1.149846 | 0.687949 |
| ILMN_1807211 | NM_032316 | 84276 | NICN1 | 0.244000 | −0.078308 | −0.734359 |
| ILMN_1815086 | NM_004148 | 4814 | NINJ1 | 0.288667 | 0.011538 | −0.781282 |
| ILMN_1735827 | NM_007184 | 11188 | NISCH | 0.261111 | −0.202615 | −0.497692 |
| ILMN_1723768 | NM_170722 | 79671 | NLRX1 | 0.336667 | −0.477385 | −0.221026 |
| ILMN_1784783 | NM_003551 | 8382 | NME5 | 0.335556 | −0.506000 | −3.051026 |
| ILMN_1710315 | NM_014697 | 9722 | NOS1AP | 0.044889 | −0.382769 | −1.746410 |
| ILMN_1783665 | NM_052946 | 115677 | NOSTRIN | 0.534667 | −0.549846 | −2.291282 |
| ILMN_1721968 | NM_002515 | 4857 | NOVA1 | −0.562000 | −1.081538 | −2.910769 |
| ILMN_1811363 | NM_006491 | 4857 | NOVA1 | −0.813111 | −1.360154 | −3.694103 |
| ILMN_1811593 | NM_207330 | 152519 | NPAL1 | −1.475333 | −1.382308 | 0.599231 |
| ILMN_1784917 | NM_015392 | 56654 | NPDC1 | 0.132222 | −0.000154 | −0.846923 |
| ILMN_1764127 | NM_207181 | 4867 | NPHP1 | −0.377778 | 0.317692 | −1.181795 |
| ILMN_1750412 | NM_025152 | 80224 | NUBPL | −0.038222 | 0.169077 | −0.840000 |
| ILMN_1781996 | NM_152395 | 131870 | NUDT16 | 0.031111 | 0.101846 | −0.691282 |
| ILMN_1712596 | NM_194289 | 152195 | NUDT16P | −0.156667 | −0.465538 | −1.938718 |
| ILMN_1787885 | NM_024815 | 79873 | NUDT18 | 0.218444 | −0.087231 | −0.900000 |
| ILMN_1714951 | NM_007083 | 11162 | NUDT6 | −0.003556 | 0.064154 | −1.194359 |
| ILMN_1780659 | NM_007083 | 11162 | NUDT6 | −0.015556 | 0.071846 | −1.023846 |
| ILMN_1673962 | NM_015135 | 23165 | NUP205 | −0.470889 | −0.088923 | 0.367179 |
| ILMN_1725612 | NM_007172 | 10762 | NUP50 | −0.397111 | −0.219231 | 0.290769 |
| ILMN_1714000 | NM_007225 | 11248 | NXPH3 | −0.171111 | −0.462615 | −2.233077 |
| ILMN_1741214 | XM_938935 | 11247 | NXPH4 | −1.536667 | −1.158923 | 0.346410 |
| ILMN_1768020 | NM_033417 | 93323 | NY-SAR-48 | −0.532222 | −0.171846 | 0.429231 |
| ILMN_1785852 | NM_001031716 | 64859 | OBFC2A | −0.207778 | −0.873538 | 0.434359 |
| ILMN_1757388 | NM_024578 | 79629 | OCEL1 | −0.067778 | 0.226000 | −0.902051 |
| ILMN_1748591 | NM_002539 | 4953 | ODC1 | −0.610444 | −0.879077 | 0.605641 |
| ILMN_1749846 | NM_005014 | 4958 | OMD | 0.118444 | −0.814308 | −2.276154 |
| ILMN_1813846 | NM_002560 | 5025 | P2RX4 | 0.202444 | −0.011846 | −0.658462 |
| ILMN_1771223 | NM_007365 | 11240 | PADI2 | −0.674444 | −0.508462 | 0.645897 |
| ILMN_1812031 | NM_002579 | 5064 | PALM | 0.182222 | −0.121385 | −1.920769 |
| ILMN_1658373 | NM_014871 | 9924 | PAN2 | 0.100667 | 0.071538 | −0.724872 |
| ILMN_1680782 | NM_152716 | 219988 | PATL1 | −0.332222 | −0.139077 | 0.291795 |
| ILMN_1651364 | NM_032151 | 84105 | PCBD2 | 0.187333 | 0.052154 | −1.282564 |
| ILMN_1724825 | NM_001098620 | 5094 | PCBP2 | 0.144889 | 0.054615 | −0.398974 |
| ILMN_1798602 | NM_015885 | 51585 | PCF11 | 0.210444 | −0.075077 | −0.357179 |
| ILMN_1690487 | NM_006197 | 5108 | PCM1 | 0.428667 | −0.282615 | −0.743590 |
| ILMN_1694177 | NM_182649 | 5111 | PCNA | −0.646222 | 0.004769 | 0.278205 |
| ILMN_1720093 | NM_174895 | 126006 | PCP2 | −0.090444 | 0.009231 | −2.193846 |
| ILMN_1769018 | NM_017573 | 54760 | PCSK4 | 0.281778 | −0.085538 | −1.584103 |
| ILMN_1693259 | NM_013374 | 10015 | PDCD6IP | 0.045556 | 0.148154 | −0.841538 |
| ILMN_1698261 | NM_000283 | 5158 | PDE6B | 0.283556 | −0.436154 | −1.570769 |
| ILMN_1705589 | NM_002603 | 5150 | PDE7A | −0.861778 | −0.306615 | 0.459744 |
| ILMN_1772369 | NM_000284 | 5160 | PDHA1 | −0.309556 | −0.143692 | 0.312051 |
| ILMN_1739274 | NM_000925 | 5162 | PDHB | 0.189111 | −0.023077 | −0.479231 |
| ILMN_1680626 | NM_005742 | 10130 | PDIA6 | −0.412444 | −0.258462 | 0.447179 |
| ILMN_1683916 | NM_002618 | 5194 | PEX13 | −0.484444 | −0.180462 | 0.418718 |
| ILMN_1755536 | NM_002624 | 5204 | PFDN5 | 0.133111 | 0.061231 | −0.485641 |
| ILMN_1672122 | NM_177938 | 54681 | PH-4 | 0.019556 | 0.214769 | −1.554615 |
| ILMN_1728380 | NM_001008489 | 493911 | PHOSPHO2 | 0.186000 | 0.027846 | −0.920513 |
| ILMN_1806924 | NM_174933 | 254295 | PHYHD1 | 0.715333 | −1.041538 | −3.558205 |
| ILMN_1738759 | NM_015937 | 51604 | PIGT | −0.043111 | 0.225846 | −0.747436 |
| ILMN_1666924 | NM_032409 | 65018 | PINK1 | 0.134667 | 0.005231 | −0.569231 |
| ILMN_1766658 | NM_182687 | 9088 | PKMYT1 | −2.098667 | 0.036308 | 0.480256 |
| ILMN_1722798 | NM_133373 | 113026 | PLCD3 | 0.162444 | −0.256615 | −1.351026 |
| ILMN_1808379 | NM_032726 | 84812 | PLCD4 | −0.709111 | 0.146462 | −2.352821 |
| ILMN_1668409 | NM_014996 | 23007 | PLCH1 | −1.968889 | −1.222769 | 0.925897 |
| ILMN_1804652 | NM_024927 | 79990 | PLEKHH3 | 0.218444 | 0.054923 | −0.833333 |
| ILMN_1787923 | NM_020376 | 57104 | PNPLA2 | 0.243111 | −0.275385 | −0.787436 |
| ILMN_1664348 | NM_004650 | 8228 | PNPLA4 | −0.070444 | 0.106462 | −2.034359 |
| ILMN_1727439 | NM_138814 | 150379 | PNPLA5 | −0.507778 | 0.094308 | 0.114872 |
| ILMN_1737704 | NM_000937 | 5430 | POLR2A | 0.268667 | −0.105077 | −0.367949 |
| ILMN_1670037 | NM_021128 | 5441 | POLR2L | 0.172444 | −0.025231 | −0.877949 |
| ILMN_1756793 | NM_006999 | 11044 | POLS | −0.444667 | −0.186615 | 0.382308 |
| ILMN_1768273 | NM_015029 | 10940 | POP1 | −0.848000 | −0.382000 | 0.611795 |
| ILMN_1674302 | NM_002703 | 5471 | PPAT | −0.512889 | 0.142923 | 0.052051 |
| ILMN_1715616 | NM_203467 | 122769 | PPIL5 | −0.736444 | 0.104923 | 0.202821 |
| ILMN_1778890 | NM_152329 | 122769 | PPIL5 | −0.708000 | 0.062615 | 0.110256 |
| ILMN_1771637 | NM_002707 | 5496 | PPM1G | −0.342889 | −0.019077 | 0.260256 |
| ILMN_1799150 | NM_005167 | 333926 | PPM1J | −0.004444 | 0.154462 | −1.867179 |
| ILMN_1651406 | NM_138689 | 26472 | PPP1R14B | −0.261333 | −0.425538 | 0.466410 |
| ILMN_1664855 | NM_030949 | 81706 | PPP1R14C | −3.222000 | −3.580000 | 0.890256 |
| ILMN_1736670 | NM_005398 | 5507 | PPP1R3C | −0.008667 | −0.868615 | −3.407179 |
| ILMN_1796962 | NM_000945 | 5534 | PPP3R1 | −0.296444 | −0.122923 | 0.290000 |
| ILMN_1777342 | NM_020820 | 57580 | PREX1 | 0.030444 | −0.072154 | −2.212308 |
| ILMN_1783388 | NM_024888 | 79948 | PRG2 | −1.018889 | −0.283538 | −3.266410 |
| ILMN_1793522 | NM_006253 | 5564 | PRKAB1 | 0.059111 | 0.091846 | −0.576923 |
| ILMN_1782403 | NM_018304 | 55771 | PRR11 | −1.737333 | −0.297538 | 0.737949 |
| ILMN_1692938 | NM_021154 | 29968 | PSAT1 | −3.569556 | −3.411077 | 0.765128 |
| ILMN_1717477 | NM_015310 | 23362 | PSD3 | 0.346000 | −1.107231 | −2.525385 |
| ILMN_1744649 | NM_002797 | 5693 | PSMB5 | −0.373778 | 0.101077 | 0.039744 |
| ILMN_1691086 | NM_016556 | 29893 | PSMC3IP | −0.419333 | 0.219846 | −0.082308 |
| ILMN_1659285 | NM_203433 | 8624 | PSMG1 | −0.911111 | −0.107385 | 0.524872 |
| ILMN_1779264 | NM_003720 | 8624 | PSMG1 | −0.932667 | −0.117231 | 0.477692 |
| ILMN_1671843 | NM_001032290 | 84722 | PSRC1 | −0.711111 | −0.137692 | 0.395385 |
| ILMN_1688753 | NM_014754 | 9791 | PTDSS1 | −0.623111 | −0.173231 | 0.416667 |
| ILMN_1681031 | NM_005859 | 5813 | PURA | 0.104889 | 0.028769 | −0.744615 |
| ILMN_1718303 | NM_002856 | 5819 | PVRL2 | 0.208889 | 0.032769 | −0.898205 |
| ILMN_1712312 | NM_004663 | 8766 | RAB11A | 0.103111 | 0.088615 | −0.477179 |
| ILMN_1701913 | NM_022449 | 64284 | RAB17 | 0.097333 | 0.033538 | −1.175128 |
| ILMN_1691143 | NM_021252 | 22931 | RAB18 | −0.293778 | 0.281692 | −0.723590 |
| ILMN_1652394 | NM_002865 | 5862 | RAB2A | −0.562889 | 0.016154 | 0.214615 |
| ILMN_1790994 | NM_014488 | 27314 | RAB30 | −0.189111 | −0.371231 | −1.618718 |
| ILMN_1750202 | NM_003929 | 8934 | RAB7L1 | −0.557333 | −0.650308 | 0.427949 |
| ILMN_1719622 | NM_001083585 | 9135 | RABEP1 | 0.346667 | −0.103231 | −1.614615 |
| ILMN_1687782 | NM_002873 | 5884 | RAD17 | 0.100444 | 0.114154 | −0.581538 |
| ILMN_1755023 | NM_133482 | 10111 | RAD50 | 0.136000 | 0.150462 | −0.762308 |
| ILMN_1659864 | NM_002875 | 5888 | RAD51 | −1.433333 | −0.005846 | 0.320256 |
| ILMN_1814464 | NM_005854 | 10266 | RAMP2 | −0.124000 | −0.241538 | −1.904359 |
| ILMN_1761782 | NM_005856 | 10268 | RAMP3 | −0.999556 | −1.467692 | −3.088974 |
| ILMN_1738913 | NM_002888 | 5918 | RARRES1 | −2.230444 | −3.300923 | 0.627692 |
| ILMN_1800091 | NM_206963 | 5918 | RARRES1 | −1.880444 | −3.168923 | 0.640513 |
| ILMN_1793517 | NM_004658 | 8437 | RASAL1 | −1.266889 | −1.336000 | 0.728205 |
| ILMN_1732127 | NM_022128 | 64080 | RBKS | 0.153778 | −0.000462 | −0.846154 |
| ILMN_1793033 | NM_018077 | 55131 | RBM28 | −0.403333 | −0.221385 | 0.383590 |
| ILMN_1688087 | NM_173587 | 283248 | RCOR2 | −0.822667 | −0.901846 | 0.740000 |
| ILMN_1682095 | NM_018254 | 55758 | RCOR3 | 0.193111 | −0.021846 | −0.706410 |
| ILMN_1810000 | NM_003708 | 8608 | RDH16 | −2.374667 | −0.151846 | −1.566410 |
| ILMN_1802380 | NM_001042682 | 473 | RERE | 0.278000 | −0.177692 | −0.462821 |
| ILMN_1732336 | NM_002914 | 5982 | RFC2 | −0.697778 | −0.190615 | 0.438205 |
| ILMN_1741005 | NM_152292 | 93587 | RG9MTD2 | 0.008222 | 0.158154 | −0.813333 |
| ILMN_1763704 | NM_183337 | 8786 | RGS11 | −0.211111 | −0.198000 | −2.167692 |
| ILMN_1669983 | NM_015668 | 26166 | RGS22 | −0.646444 | −0.436462 | −3.193846 |
| ILMN_1657949 | NM_005614 | 6009 | RHEB | −0.396222 | −0.062000 | 0.287949 |
| ILMN_1663532 | NM_018157 | 55188 | RIC8B | −0.057778 | 0.139077 | −0.489744 |
| ILMN_1758939 | NM_003821 | 8767 | RIPK2 | −0.683333 | −0.295692 | 0.475897 |
| ILMN_1656335 | NM_006912 | 6016 | RIT1 | −0.507111 | −0.233692 | 0.415897 |
| ILMN_1696974 | NM_194430 | 6038 | RNASE4 | 0.147333 | −0.195231 | −1.124615 |
| ILMN_1776602 | NM_194431 | 6038 | RNASE4 | 0.246667 | −0.290462 | −1.407179 |
| ILMN_1714461 | NM_183399 | 9604 | RNF14 | −0.147333 | 0.208462 | −0.495641 |
| ILMN_1719951 | NM_144726 | 153830 | RNF145 | −0.782667 | −1.348615 | 0.539231 |
| ILMN_1805614 | NM_134261 | 6095 | RORA | 0.298667 | −0.230615 | −1.233077 |
| ILMN_1693717 | NM_006987 | 9501 | RPH3AL | −0.098667 | 0.071538 | −1.661795 |
| ILMN_1709039 | NM_033251 | 6137 | RPL13 | −1.161333 | −0.175231 | 0.351795 |
| ILMN_1713369 | NM_012423 | 23521 | RPL13A | 0.280889 | −0.160462 | −0.656154 |
| ILMN_1762747 | NM_002948 | 6138 | RPL15 | 0.105333 | 0.013846 | −0.530513 |
| ILMN_1710001 | NM_001035267 | 6171 | RPL41 | −0.973556 | 0.146769 | −0.209744 |
| ILMN_1725656 | NM_000969 | 6125 | RPL5 | 0.159778 | −0.220615 | −0.004615 |
| ILMN_1712678 | NM_015920 | 51065 | RPS27L | 0.174000 | 0.031846 | −0.647436 |
| ILMN_1699772 | NM_021244 | 58528 | RRAGD | −0.903333 | −1.100154 | 0.554103 |
| ILMN_1791097 | NM_018364 | 54665 | RSBN1 | 0.194889 | −0.118462 | −0.358462 |
| ILMN_1682494 | NM_016625 | 51319 | RSRC1 | −0.538889 | −0.056615 | 0.286154 |
| ILMN_1687326 | NM_206852 | 6252 | RTN1 | −0.272222 | −2.305846 | −4.979487 |
| ILMN_1756928 | NM_021136 | 6252 | RTN1 | 0.383333 | −1.423385 | −2.393590 |
| ILMN_1749115 | NM_206901 | 6253 | RTN2 | 0.090000 | −0.254923 | −1.383846 |
| ILMN_1748983 | NM_007008 | 57142 | RTN4 | −0.052444 | −0.606769 | −2.748462 |
| ILMN_1798465 | NM_001005861 | 6259 | RYK | 0.130889 | −0.291231 | 0.046410 |
| ILMN_1752793 | NM_005870 | 10284 | SAP18 | −0.078667 | 0.152462 | −0.948974 |
| ILMN_1728907 | NM_004866 | 9522 | SCAMP1 | 0.105556 | 0.043231 | −0.413846 |
| ILMN_1795839 | NM_016002 | 51097 | SCCPDH | 0.045778 | 0.070308 | −1.858205 |
| ILMN_1767470 | NM_021626 | 59342 | SCPEP1 | −0.245778 | −0.944769 | 0.476410 |
| ILMN_1662016 | NM_138355 | 90507 | SCRN2 | 0.128444 | −0.066769 | −0.725128 |
| ILMN_1726496 | NM_005065 | 6400 | SEL1L | 0.079111 | 0.010923 | −0.695385 |
| ILMN_1746368 | NM_016275 | 51714 | SELT | 0.048444 | 0.207846 | −0.890000 |
| ILMN_1750092 | NM_153825 | 51091 | SEPSECS | 0.005556 | 0.187538 | −0.864872 |
| ILMN_1659953 | NM_019106 | 55964 | SEPT3 | −1.989778 | −0.697231 | 0.660769 |
| ILMN_1746673 | NM_019106 | 55964 | SEPT3 | −2.756667 | −1.037846 | 0.701026 |
| ILMN_1801934 | NM_013368 | 29946 | SERTAD3 | −0.005111 | 0.269538 | −0.824872 |
| ILMN_1724504 | NM_032233 | 84193 | SETD3 | 0.206222 | −0.121385 | −0.304615 |
| ILMN_1761996 | NM_006925 | 6430 | SFRS5 | 0.433111 | −0.229077 | −0.541795 |
| ILMN_1795976 | NM_178858 | 118980 | SFXN2 | −0.022889 | 0.083538 | −1.484103 |
| ILMN_1746699 | NM_152524 | 151246 | SGOL2 | −0.833111 | 0.008154 | 0.404872 |
| ILMN_1779171 | NM_014853 | 9905 | SGSM2 | 0.380222 | −0.278923 | −0.666154 |
| ILMN_1762540 | NM_018130 | 55164 | SHQ1 | 0.085556 | 0.028154 | −0.423077 |
| ILMN_1763442 | NM_020717 | 57477 | SHROOM4 | 0.469778 | −0.784615 | −0.643077 |
| ILMN_1736965 | NM_021805 | 59307 | SIGIRR | −0.046000 | −0.017077 | −1.406410 |
| ILMN_1807981 | XM_001129013 | 59307 | SIGIRR | 0.061778 | 0.095385 | −0.842051 |
| ILMN_1678729 | NM_001037633 | 64374 | SIL1 | 0.160222 | −0.072154 | −0.584103 |
| ILMN_1711766 | NM_006930 | 6500 | SKP1A | −0.074889 | 0.284923 | −0.732051 |
| ILMN_1665538 | NM_032637 | 6502 | SKP2 | −0.796000 | −0.351077 | 0.627692 |
| ILMN_1791002 | NM_005983 | 6502 | SKP2 | −1.428222 | −0.543077 | 0.758462 |
| ILMN_1782938 | NM_018593 | 117247 | SLC16A10 | −1.519778 | −1.678000 | 0.460513 |
| ILMN_1698996 | NM_194255 | 6573 | SLC19A1 | −0.619778 | 0.010923 | 0.259487 |
| ILMN_1815581 | NM_183233 | 5002 | SLC22A18 | 0.129111 | 0.001385 | −1.011026 |
| ILMN_1699357 | NM_003060 | 6584 | SLC22A5 | 0.122000 | 0.130000 | −1.044615 |
| ILMN_1747395 | NM_004727 | 9187 | SLC24A1 | 0.152889 | 0.017077 | −0.681795 |
| ILMN_1668012 | NM_014251 | 10165 | SLC25A13 | −0.604000 | −0.204462 | 0.367949 |
| ILMN_1768251 | NM_173471 | 115286 | SLC25A26 | 0.081111 | 0.028923 | −0.519231 |
| ILMN_1724612 | NM_201520 | 399512 | SLC25A35 | 0.153556 | −0.101538 | −1.847179 |
| ILMN_1781231 | NM_017875 | 54977 | SLC25A38 | −0.066444 | 0.124615 | −0.675128 |
| ILMN_1802348 | NM_152313 | 120103 | SLC36A4 | −0.912667 | −0.822769 | 0.539744 |
| ILMN_1745770 | NM_007231 | 11254 | SLC6A14 | −2.646000 | −4.189692 | −0.035897 |
| ILMN_1723287 | NM_014037 | 28968 | SLC6A16 | −2.154222 | −1.563538 | 0.476410 |
| ILMN_1781400 | NM_001008539 | 6542 | SLC7A2 | −0.304667 | −0.373231 | −4.946154 |
| ILMN_1774229 | NM_004173 | 6545 | SLC7A4 | 0.200889 | −1.182154 | −2.225128 |
| ILMN_1807894 | NM_182728 | 23428 | SLC7A8 | 0.256444 | −0.029231 | −1.992051 |
| ILMN_1783120 | NM_007159 | 7871 | SLMAP | 0.200000 | −0.096462 | −0.403077 |
| ILMN_1803522 | NM_016045 | 51012 | SLMO2 | −0.556222 | 0.240615 | −0.185897 |
| ILMN_1742224 | NM_024755 | 79811 | SLTM | 0.078667 | 0.006769 | −0.520769 |
| ILMN_1705080 | NM_020427 | 57152 | SLURP1 | −1.780222 | −1.373077 | 0.265128 |
| ILMN_1674551 | NM_005903 | 4090 | SMAD5 | 0.016000 | 0.056154 | −0.520769 |
| ILMN_1719641 | NM_022138 | 64094 | SMOC2 | −0.002222 | −0.601385 | −2.924103 |
| ILMN_1804642 | NM_014311 | 23583 | SMUG1 | −0.034000 | 0.169538 | −0.618974 |
| ILMN_1721605 | NM_020197 | 56950 | SMYD2 | −0.486222 | −0.260462 | 0.436923 |
| ILMN_1698478 | NM_003083 | 6618 | SNAPC2 | 0.154667 | 0.072462 | −0.675897 |
| ILMN_1771060 | NM_177542 | 6633 | SNRPD2 | −0.596444 | 0.035231 | 0.172821 |
| ILMN_1683562 | NM_003096 | 6637 | SNRPG | −0.208000 | −0.146000 | 0.276667 |
| ILMN_1804051 | NM_013321 | 29886 | SNX8 | −0.539111 | −0.171385 | 0.350513 |
| ILMN_1773459 | NM_003108 | 6664 | SOX11 | −3.042222 | −2.898000 | 1.078974 |
| ILMN_1687247 | NM_022827 | 64847 | SPATA20 | −0.038667 | −0.069692 | −1.209231 |
| ILMN_1665280 | NM_014041 | 28972 | SPCS1 | 0.095333 | 0.086462 | −0.459744 |
| ILMN_1678391 | NM_144722 | 79925 | SPEF2 | −0.035556 | 0.135692 | −1.046923 |
| ILMN_1729281 | NM_020126 | 56848 | SPHK2 | 0.185778 | −0.009385 | −0.516923 |
| ILMN_1735250 | NM_032840 | 84926 | SPRYD3 | 0.056444 | 0.164154 | −0.700513 |
| ILMN_1793241 | NM_001047 | 6715 | SRD5A1 | −1.378889 | −0.859231 | 0.575641 |
| ILMN_1657451 | NM_182691 | 6733 | SRPK2 | 0.159556 | −0.202615 | −1.312051 |
| ILMN_1755234 | NM_017857 | 54961 | SSH3 | 0.238667 | −0.002308 | −0.817179 |
| ILMN_1681245 | NM_021978 | 6768 | ST14 | −0.421333 | −0.755385 | 0.614615 |
| ILMN_1717052 | NM_006645 | 10809 | STARD10 | −0.190889 | 0.161692 | −1.665385 |
| ILMN_1665311 | NM_001007532 | 246744 | STH | 0.193333 | −0.201385 | −1.676154 |
| ILMN_1807232 | NM_003035 | 6491 | STIL | −0.984444 | −0.114000 | 0.574359 |
| ILMN_1657796 | NM_203401 | 3925 | STMN1 | −1.067778 | −0.257538 | 0.603846 |
| ILMN_1745593 | NM_005563 | 3925 | STMN1 | −1.086667 | −0.383385 | 0.546410 |
| ILMN_1736054 | NM_006713 | 10923 | SUB1 | −0.189556 | 0.008769 | −1.107179 |
| ILMN_1652379 | NM_003848 | 8801 | SUCLG2 | 0.132000 | −0.017231 | −0.532308 |
| ILMN_1803745 | NM_000456 | 6821 | SUOX | 0.176444 | 0.012615 | −0.915128 |
| ILMN_1781479 | NM_003173 | 6839 | SUV39H1 | −0.563556 | −0.008154 | 0.178718 |
| ILMN_1771261 | NM_030786 | 81493 | SYNC1 | 0.292444 | −0.146462 | −1.331795 |
| ILMN_1727740 | NM_006372 | 10492 | SYNCRIP | −0.454444 | −0.181231 | 0.354103 |
| ILMN_1728496 | NM_175733 | 143425 | SYT9 | −0.180222 | −0.439692 | −2.099231 |
| ILMN_1750785 | NM_032872 | 84958 | SYTL1 | 0.172889 | −0.035846 | −0.976667 |
| ILMN_1651428 | NM_032379 | 54843 | SYTL2 | −0.292222 | −0.047692 | −1.752564 |
| ILMN_1682929 | NM_206930 | 54843 | SYTL2 | 0.119111 | −0.086462 | −1.660000 |
| ILMN_1720623 | NM_001009991 | 94120 | SYTL3 | −0.442667 | −0.678000 | 0.505897 |
| ILMN_1719599 | NM_080737 | 94121 | SYTL4 | 0.203333 | −0.208154 | −2.054615 |
| ILMN_1694888 | NM_003184 | 6873 | TAF2 | −0.455111 | 0.025692 | 0.143590 |
| ILMN_1683948 | NM_001025247 | 27097 | TAF5L | −0.484000 | −0.106462 | 0.271026 |
| ILMN_1693882 | NM_153365 | 202018 | TAPT1 | −0.082444 | 0.124154 | −0.885641 |
| ILMN_1666498 | NM_152295 | 6897 | TARS | −0.480444 | −0.078769 | 0.264615 |
| ILMN_1692844 | NM_018317 | 55296 | TBC1D19 | 0.092667 | 0.056308 | −0.681282 |
| ILMN_1703891 | NM_015130 | 23158 | TBC1D9 | 0.164000 | 0.181692 | −2.462821 |
| ILMN_1665526 | NM_198723 | 6919 | TCEA2 | 0.096000 | 0.141077 | −0.857692 |
| ILMN_1768815 | NM_003195 | 6919 | TCEA2 | −0.047778 | 0.151385 | −1.077436 |
| ILMN_1749478 | NM_032926 | 85012 | TCEAL3 | 0.049556 | 0.172769 | −1.398974 |
| ILMN_1748625 | NM_001006937 | 79921 | TCEAL4 | 0.076222 | 0.146154 | −1.159231 |
| ILMN_1799099 | NM_031898 | 64518 | TEKT3 | 0.308889 | −0.428615 | −0.882821 |
| ILMN_1685042 | NM_015319 | 23371 | TENC1 | 0.478667 | −0.269231 | −1.370000 |
| ILMN_1765246 | NM_152829 | 26136 | TES | −0.419111 | −0.722923 | 0.511282 |
| ILMN_1653529 | NM_017746 | 54881 | TEX10 | −0.477111 | −0.376000 | 0.566410 |
| ILMN_1781623 | NM_015926 | 51368 | TEX264 | 0.034667 | 0.062769 | −0.558974 |
| ILMN_1709044 | NM_021809 | 60436 | TGIF2 | −0.694444 | −0.074000 | 0.271282 |
| ILMN_1746737 | NM_024817 | 79875 | THSD4 | −0.180000 | 0.108154 | −1.237179 |
| ILMN_1737168 | NM_024328 | 79178 | THTPA | 0.022889 | 0.103385 | −0.842821 |
| ILMN_1781408 | NM_199298 | 29087 | THYN1 | 0.236444 | −0.451231 | −0.032051 |
| ILMN_1690066 | NM_145715 | 166815 | TIGD2 | −0.546667 | −0.512462 | 0.665128 |
| ILMN_1703984 | NM_030953 | 81789 | TIGD6 | 0.120444 | 0.057385 | −0.810256 |
| ILMN_1722239 | NM_004085 | 1678 | TIMM8A | −0.537556 | −0.100615 | 0.263333 |
| ILMN_1761939 | NM_017858 | 54962 | TIPIN | −0.616444 | −0.098308 | 0.350769 |
| ILMN_1751572 | NM_005077 | 7088 | TLE1 | −0.420889 | −1.029538 | 0.551282 |
| ILMN_1679798 | NM_017442 | 54106 | TLR9 | −0.739778 | −0.142462 | 0.330513 |
| ILMN_1789970 | NM_006405 | 10548 | TM9SF1 | 0.050444 | 0.140000 | −0.567949 |
| ILMN_1664750 | NM_016056 | 51643 | TMBIM4 | 0.214222 | 0.100615 | −0.872821 |
| ILMN_1693311 | NM_003217 | 7009 | TMBIM6 | 0.020222 | 0.190923 | −0.619744 |
| ILMN_1724139 | NM_052932 | 114908 | TMEM123 | −0.282444 | −0.793692 | 0.592051 |
| ILMN_1663033 | NM_138385 | 92305 | TMEM129 | 0.061333 | 0.040308 | −0.627692 |
| ILMN_1708110 | NM_018342 | 55314 | TMEM144 | 0.208889 | −0.025846 | −1.020769 |
| ILMN_1789112 | NM_173633 | 284339 | TMEM145 | −0.816222 | −0.680923 | −2.887436 |
| ILMN_1807580 | NM_153354 | 153396 | TMEM161B | −0.080444 | 0.138769 | −0.423333 |
| ILMN_1654629 | NM_032326 | 84286 | TMEM175 | 0.120444 | −0.004462 | −0.595641 |
| ILMN_1789732 | NM_199129 | 387521 | TMEM189 | −0.610444 | −0.088923 | 0.213590 |
| ILMN_1725880 | NM_015257 | 23306 | TMEM194 | −0.585111 | 0.162769 | 0.082821 |
| ILMN_1809639 | NM_178505 | 219623 | TMEM26 | 0.359778 | −0.998923 | −2.254615 |
| ILMN_1678004 | NM_015012 | 440026 | TMEM41B | 0.182222 | 0.009692 | −0.553333 |
| ILMN_1674985 | NM_018022 | 55092 | TMEM51 | −0.434444 | −0.318000 | 0.429744 |
| ILMN_1780141 | NM_016127 | 51669 | TMEM66 | 0.206889 | −0.071077 | −0.483333 |
| ILMN_1665876 | NM_173610 | 283673 | TMEM84 | −0.012667 | −0.850615 | −2.490256 |
| ILMN_1710962 | NM_014573 | 27346 | TMEM97 | −0.814444 | 0.018000 | 0.220256 |
| ILMN_1689979 | NM_020644 | 56674 | TMEM9B | 0.184222 | 0.068615 | −0.635385 |
| ILMN_1697409 | NM_003820 | 8764 | TNFRSF14 | 0.348889 | −0.294154 | −0.550256 |
| ILMN_1664071 | NM_000364 | 7139 | TNNT2 | −1.065778 | −0.960308 | 0.623333 |
| ILMN_1765523 | NM_019009 | 54472 | TOLLIP | 0.130000 | −0.148462 | −0.822051 |
| ILMN_1743131 | NM_014828 | 9878 | TOX4 | −0.065111 | 0.144308 | −0.509744 |
| ILMN_1790350 | NM_198485 | 285386 | TPRG1 | −0.279778 | −1.404462 | −4.264103 |
| ILMN_1754629 | NM_014965 | 22906 | TRAK1 | 0.135556 | −0.001231 | −0.893590 |
| ILMN_1745079 | NM_015271 | 23321 | TRIM2 | −1.125778 | −1.867692 | 0.527436 |
| ILMN_1687703 | NM_015294 | 4591 | TRIM37 | −0.488444 | 0.117538 | −0.036154 |
| ILMN_1674533 | NM_018646 | 55503 | TRPV6 | −2.174222 | −2.951385 | 0.378205 |
| ILMN_1747546 | NM_005727 | 10103 | TSPAN1 | −0.551778 | −0.151077 | −2.943846 |
| ILMN_1669881 | NM_014399 | 27075 | TSPAN13 | −0.200667 | 0.235385 | −1.129231 |
| ILMN_1725079 | NM_005981 | 6302 | TSPAN31 | 0.078000 | 0.047538 | −0.942051 |
| ILMN_1696757 | NM_001042601 | 151613 | TTC14 | 0.095556 | 0.003692 | −0.711026 |
| ILMN_1784516 | NM_145170 | 118491 | TTC18 | 0.176222 | −0.341538 | −1.684103 |
| ILMN_1715505 | NM_001007795 | 115669 | TTC6 | −0.073333 | −0.231538 | −1.988974 |
| ILMN_1652309 | NM_198310 | 123016 | TTC8 | 0.191333 | −0.050000 | −0.736154 |
| ILMN_1746846 | NM_014640 | 9654 | TTLL4 | −0.927111 | −1.058000 | 0.945897 |
| ILMN_1786212 | NM_177987 | 347688 | TUBB8 | −0.414444 | 0.020000 | 0.181026 |
| ILMN_1701052 | NM_016437 | 27175 | TUBG2 | 0.049333 | 0.052000 | −0.978462 |
| ILMN_1804329 | NM_007275 | 11334 | TUSC2 | 0.008444 | 0.173231 | −0.651795 |
| ILMN_1691272 | NM_006545 | 10641 | TUSC4 | 0.143333 | 0.085077 | −0.501538 |
| ILMN_1343293 | NM_003329 | | TXN | −0.402222 | −0.041385 | 0.275897 |
| ILMN_1680314 | NM_003329 | 7295 | TXN | −0.483778 | −0.041692 | 0.283846 |
| ILMN_1662848 | NM_024715 | 79770 | TXNDC15 | 0.017111 | 0.118308 | −0.581026 |
| ILMN_1663099 | NM_003337 | 7320 | UBE2B | −0.060889 | 0.196154 | −1.063333 |
| ILMN_1712525 | NM_006357 | 10477 | UBE2E3 | −0.466667 | −1.478615 | 0.599487 |
| ILMN_1726107 | NM_001032288 | 7335 | UBE2V1 | −0.651333 | 0.123077 | −0.059744 |
| ILMN_1770515 | NM_003350 | 7336 | UBE2V2 | −0.620222 | 0.072000 | 0.188205 |
| ILMN_1764549 | NM_000462 | 7337 | UBE3A | 0.269333 | −0.098462 | −0.527436 |
| ILMN_1752027 | NM_130466 | 89910 | UBE3B | −0.055333 | 0.215231 | −0.583846 |
| ILMN_1726798 | NM_152376 | 127733 | UBXN10 | 0.237111 | −0.058923 | −1.099744 |
| ILMN_1665737 | NM_001035247 | 7353 | UFD1L | −0.722000 | −0.011692 | 0.213077 |
| ILMN_1736939 | NM_003358 | 7357 | UGCG | −0.005111 | 0.046154 | −1.746667 |
| ILMN_1729563 | NM_003359 | 7358 | UGDH | 0.027778 | −0.136308 | −1.445385 |
| ILMN_1786065 | NM_013282 | 29128 | UHRF1 | −1.289333 | 0.087846 | 0.282308 |
| ILMN_1771396 | NM_025217 | 80328 | ULBP2 | −1.659556 | −1.420462 | 0.637436 |
| ILMN_1759453 | NM_006294 | 7381 | UQCRB | −0.942444 | 0.065538 | 0.190513 |
| ILMN_1659523 | NM_006590 | 10713 | USP39 | −0.278222 | −0.023846 | 0.212564 |
| ILMN_1722953 | NM_017944 | 55031 | USP47 | 0.014000 | 0.039846 | −0.769744 |
| ILMN_1745499 | NM_153477 | 8409 | UXT | −0.413778 | −0.092154 | 0.329487 |
| ILMN_1705310 | NM_007146 | 7716 | VEZF1 | 0.145333 | 0.020462 | −0.550769 |
| ILMN_1757497 | NM_003378 | 7425 | VGF | −3.194667 | −1.829077 | −0.627949 |
| ILMN_1767691 | NM_032353 | 84313 | VPS25 | −0.224444 | 0.259692 | −0.597179 |
| ILMN_1673555 | NM_015289 | 23339 | VPS39 | 0.130444 | 0.050154 | −0.521026 |
| ILMN_1805828 | NM_003384 | 7443 | VRK1 | −0.611333 | −0.120154 | 0.379231 |
| ILMN_1707502 | NM_015426 | 25886 | WDR51A | −1.249556 | 0.080615 | 0.247692 |
| ILMN_1655203 | NM_182627 | 348793 | WDR53 | −0.400444 | 0.010462 | 0.233077 |
| ILMN_1744240 | NM_145647 | 93594 | WDR67 | −0.828222 | −0.033385 | 0.277949 |
| ILMN_1744611 | NM_015420 | 25879 | WDSOF1 | −1.192000 | −0.172615 | 0.297179 |
| ILMN_1669114 | NM_032387 | 65266 | WNK4 | −0.313333 | −0.464154 | −3.748718 |
| ILMN_1771057 | NM_020196 | 56949 | XAB2 | 0.015111 | 0.077231 | −0.667436 |
| ILMN_1759495 | NM_020750 | 57510 | XPO5 | −0.523111 | −0.098000 | 0.396410 |
| ILMN_1676899 | NM_018023 | 55689 | YEATS2 | −0.511778 | −0.408462 | 0.665897 |
| ILMN_1782444 | NM_032312 | 84272 | YIPF4 | −0.325778 | −0.206462 | 0.383590 |
| ILMN_1750145 | NM_012479 | 7532 | YWHAG | −0.738000 | 0.082615 | 0.125641 |
| ILMN_1674385 | NM_006826 | 10971 | YWHAQ | −0.220667 | −0.174308 | 0.342308 |
| ILMN_1782129 | NM_014838 | 9889 | ZBED4 | −0.319778 | −0.348923 | 0.469231 |
| ILMN_1795905 | NM_020899 | 57659 | ZBTB4 | 0.216444 | −0.023846 | −0.714872 |
| ILMN_1699440 | NM_145166 | 92999 | ZBTB47 | 0.203556 | −0.122308 | −0.607179 |
| ILMN_1785292 | NM_024824 | 79882 | ZC3H14 | 0.268667 | 0.011231 | −0.682051 |
| ILMN_1679984 | NM_173798 | 170261 | ZCCHC12 | −0.707556 | 0.023077 | 0.107436 |
| ILMN_1659082 | NM_033114 | 85437 | ZCRB1 | −0.034889 | 0.273846 | −1.105385 |
| ILMN_1686099 | NM_021260 | 53349 | ZFYVE1 | 0.281778 | −0.154462 | −0.456667 |
| ILMN_1661010 | NM_001011656 | 84460 | ZMAT1 | 0.252444 | −0.123077 | −0.941538 |
| ILMN_1790574 | NM_015896 | 51364 | ZMYND10 | 0.396667 | −0.176154 | −2.518205 |
| ILMN_1757627 | NM_138462 | 116225 | ZMYND19 | −0.323778 | −0.080154 | 0.305641 |
| ILMN_1758643 | NM_144680 | 7566 | ZNF18 | 0.300889 | −0.298769 | −0.539487 |
| ILMN_1670377 | NM_021143 | 7568 | ZNF20 | 0.276222 | −0.055692 | −0.601026 |
| ILMN_1728230 | NM_001099437 | 90075 | ZNF30 | 0.116889 | 0.074615 | −0.808718 |
| ILMN_1772876 | NM_018660 | 55893 | ZNF395 | 0.425556 | −0.352462 | −0.720769 |
| ILMN_1799529 | NM_152355 | 126068 | ZNF441 | 0.230000 | −0.032154 | −0.860000 |
| ILMN_1663754 | NM_030824 | 79973 | ZNF442 | −0.031111 | 0.087385 | −1.324872 |
| ILMN_1743767 | NM_017908 | 55663 | ZNF446 | 0.128667 | 0.122000 | −0.651538 |
| ILMN_1683854 | NM_001007101 | 83744 | ZNF484 | 0.244667 | 0.022615 | −0.603846 |
| ILMN_1681846 | NM_152606 | 163255 | ZNF540 | 0.424444 | −0.495231 | −1.199744 |
| ILMN_1709661 | NM_145276 | 147837 | ZNF563 | −0.207556 | −0.032000 | −1.233846 |
| ILMN_1712798 | NM_020747 | 57507 | ZNF608 | 0.218667 | −1.313846 | −0.682821 |
| ILMN_1738046 | NM_014789 | 9831 | ZNF623 | −0.791556 | 0.225231 | −0.152821 |
| ILMN_1713454 | NM_024833 | 79891 | ZNF671 | 0.280444 | −0.379538 | −1.239487 |
| ILMN_1736577 | NM_001024683 | 146542 | ZNF688 | 0.033111 | 0.115385 | −0.647179 |
| ILMN_1747943 | NM_020394 | 57116 | ZNF695 | −1.583333 | −0.955231 | 0.974872 |
| ILMN_1805271 | NM_133474 | 170960 | ZNF721 | 0.031333 | 0.095077 | −0.476154 |
| ILMN_1740193 | NM_001004304 | 283337 | ZNF740 | 0.232889 | −0.027231 | −0.780513 |
| ILMN_1669696 | NM_175872 | 126375 | ZNF792 | 0.158444 | −0.011077 | −0.520256 |
| ILMN_1653163 | NM_001007072 | 54993 | ZSCAN2 | −0.658667 | −0.289077 | 0.336154 |

### Example 3

A 803-gene signature called ClinicoMolecular Triad Classification (CMTC) was designed that is applies to all BCs regardless of receptor status and has flexible tissue requirement, allows for simple clinical integration, is personalized, prognostic, and predictive of treatment response. CMTC can use fine needle aspirates at the time of initial diagnostic biopsy and Illumina whole-genome DNA to classify all BCs into 3 groups that align well with how oncologists would classify BCs (simple clinical integration). CMTC outperformed all other gene signatures in predicting prognosis and treatment response.

The genome-wide approach enables highly personalized “portfolios” that incorporate prognostic patterns of other gene signatures and oncogenic pathways, and has multiplatform compatibility. Having CMTC available at initial diagnosis allows early treatment planning, a feature that is useful especially with increasing use of pre-operative chemotherapy to improve breast conservation in selected patients.

CMTC was designed to reproduce the way oncologists currently classify BCs when making treatment decisions to simplify clinical integration of the molecular classification. An advantage is the ability to use fine needle aspiration which can be done at the time of diagnostic biopsy. Unlike Oncotype DX, CMTC can apply to all BCs and does not require pre-determination of pathologic parameters like estrogen receptor and nodal status. With CMTC, oncologists can lay out a treatment plan at diagnosis, which can be important in deciding an increasingly common treatment strategy that uses pre-operative chemotherapy to shrink larger tumours to facilitate breast conservation. CMTC was able to identify individual responders of endocrine therapy and pre-operative chemotherapy.

The versatility of a genome-wide approach allows us to combine the predictive pattern of multiple gene signatures and oncogenic pathways into highly personalized “portfolios” that predict treatments based on the biological processes involved rather than individual biomarkers. It also enables multiplatform compatibility and the potential to integrate future knowledge of the disease and treatment.

In the most recent Cancer Trends Progress Report in USA (2009/2010 update)⁷, BC was ranked the first among all cancers in national expenditures for cancer care, totalling US$13.9B in 2006, 14.8% (US$2B) of which was spent on chemotherapy alone, with an additional US$12.1 B in lost productivity (indirect costs). The lack of a test to accurately discriminate responders from non-responders of a cancer treatment often leads to over-prescription to give patients “the benefit of the doubt”, and not to take away any chance that we may be able to help them. Based on standard clinicopathological prognostic systems, only a dismal 2% absolute survival benefit can be attributed to the chemotherapy prescribed for early BCs between ages 50-59⁸ and a 5.6% absolute survival benefit for tamoxifen prescribed for node-negative, estrogen receptor-positive BC⁹.

### Example 4

Reproducibility of the Classification is Demonstrated with a Prospective Cohort of Patients.

### Background:

Numerous gene signatures have claimed prognostic significance in BCs. Each of these gene signatures was designed to answer a specific clinical or biological question, often by dichotomizing the targeted populations into a good and a bad risk group. None of these gene signatures on its own has sufficient degree of complexity to fully characterize this very heterogenous group of diseases, and hence lacks the flexibility to personalize treatments. To exploit the full potential of the genomic approach, an 803-gene molecular classification was developed, termed ClinicoMolecular Triad Classification (CMTC) that categorized BCs into 3 clinical treatment groups (triad) that can serve as a basic framework to guide management. CMTC also provide a detailed “portfolio” of 14 other gene signatures and 19 oncogenic pathways to allow further customization of the treatments. The ability to get CMTC portfolio results at the time of initial diagnosis offers the unique advantage of early treatment planning, including the use of pre-operative chemotherapy to improve breast conservation in selected patients. This study aimed to validate the CMTC classification using an independent BC cohort.

### Study Design/Results:

RNA from fine needle aspirates were collected in a prospective BC cohort (n=340) between 2008 and 2010 at Princess Margaret Hospital and Mount Sinai Hospital, Toronto. All newly diagnosed BC patients going for surgery who consented to join the study were included. DNA microarray analyses were carried out using genome-wide Illumina Human Ref-8 version 3 Beadarrays, which contained >24K oligonucleotide probes. After excluding tumors with low RNA yield (n=8, success rate 97%), non-invasive cancers (n=27), insufficient follow-up data (n=21), CMTC divided the remaining 284 BCs into 3 similar sized groups (triad). At a median follow-up of 32 months (range 6.3-52 months), the short-term recurrence was significantly worse (P=0.0048) in the poor prognostic groups. This result was similar to using an independent external validation cohort (n=2100) with long-term follow-up reported before, CMTC outperformed all other gene signatures in predicting prognosis and treatment response.

### Conclusion:

This prospective validation cohort study demonstrated reproducibility of CMTC in classifying BCs into the three major treatment groups and its prognostic significance. CMTC can be used as a platform to personalize treatments: CMTC-1 BCs (ER+, low proliferation) in general can be treated with surgery and tamoxifen alone. CMTC-2 tumours (ER+, high proliferation) will require additional treatments, including chemotherapy, in addition to tamoxifen; other biologics can be prescribed based on the activities of additional oncogenic pathways. Neo-adjuvant chemotherapy should be considered for CMTC-3 tumours (triple negative and HER2+) with addition of trastuzumab in those that show activation of the HER2 pathway.

### Example 5

TABLE 10
CMTC classification is reproducible by using different genome-
wide microarray platforms and various subsets of the 803-genes.

| | CMTC (original) | | CMTC 2012 | |
|---|---|---|---|---|
| Microarray Platform | Probe | Genes | Probe | Genes |
| Illumina HumanRef-8 V2 | 828 | 803 | 828 | 804 |
| Agilent Human 25K | 893 | 636 | 791 | 624 |
| Agilent Human 1A UNC | 909 | 656 | 909 | 668 |
| custom | | | | |
| Affymetrix Human U133 A | 945 | 529 | 949 | 534 |
| Affymetrix Human U133 A | 1606 | 741 | 1634 | 747 |
| and B | | | | |
| Affymetrix Human U133 | 1805 | 756 | 1832 | 762 |
| Plus2 | | | | |

Over time, probes get verified and gene names can be assigned/re-assigned to different probes in any of the genome wide platforms. For Illumina, the original chip (V2) used in the analysis had a slight change in the number of named genes for the 828 probes used in the original analyses (see table 10).

The updated gene sets in the other platforms were re-examined to confirm that they like the original genes in these platforms could reproduce CMTC classification. In the reanalysis, different subsets of genes were found to overlap with the genes representing the 804 genes in the 2012 version of the Illumina V2 chip. Accordingly, it is demonstrated herein that 10 different subsets of different numbers of the genes listed in Table 9 can reproduce the CMTC classification.

Accordingly it is clear that any genome-wide platform can be used to reproduce the CMTC classification, irregardless of how many genes overlapped with CMTC as long as the genes selected divide BCs into 3 treatment groups (triad) by pooling TN and Her2+ tumors together as the starting point.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

### CITATIONS FOR REFERENCES REFERRED TO IN THE SPECIFICATION

1. Polyak K: Breast cancer: origins and evolution. J Clin Invest 2007, 117:3155-3163.
2. Van Belle V, Van Calster B, Brouckaert O, Vanden Bempt I, Pintens S, Harvey V, Murray P, Naume B, Wiedswang G, Paridaens R, Moerman P, Amant F, Leunen K, Smeets A, Drijkoningen M, Wildiers H, Christiaens M R, Vergote I, Van Huffel S, Neven P: Qualitative assessment of the progesterone receptor and HER2 improves the Nottingham Prognostic Index up to 5 years after breast cancer diagnosis. J Clin Oncol 2010, 28:4129-4134.
3. Cleator S, Heller W, Coombes R C: Triple-negative breast cancer: therapeutic options. Lancet Oncol 2007, 8:235-244.
4. Gusterson B: Do ‘basal-like’ breast cancers really exist? Nat Rev Cancer 2009, 9:128-134.
5. Rakha E A, Reis-Filho J S, Ellis I O: Basal-like breast cancer: a critical review. J Clin Oncol 2008, 26:2568-2581.
6. Sørlie T, Perou C M, Tibshirani R, Aas T, Geisler S, Johnsen H, Hastie T, Eisen M B, van de Rijn M, Jeffrey S S, Thorsen T, Quist H, Matese J C, Brown P O, Botstein D, Eystein Lçnning P, Bçrresen-Dale A L: Gene expression patterns of breast carcinomas distinguish tumor subclasses with clinical implications. Proc Natl Acad Sci USA 2001, 98:10869-10874.
7. Sçrlie T, Tibshirani R, Parker J, Hastie T, Marron J S, Nobel A, Deng S, Johnsen H, Pesich R, Geisler S, Demeter J, Perou CM, Lçnning P E, Brown P O, Bçrresen-Dale A L, Botstein D: Repeated observation of breast tumor subtypes in independent gene expression data sets. Proc Natl Acad Sci USA 2003, 100:8418-8423.
8. Foulkes W D, Smith I E, Reis-Filho J S: Triple-negative breast cancer. N Engl J Med 2010, 363:1938-1948.
9. Esteva F J, Yu D, Hung M C, Hortobagyi G N: Molecular predictors of response to trastuzumab and lapatinib in breast cancer. Nat Rev Clin Oncol 2010, 7:98-107.
10. Carey L A, Dees E C, Sawyer L, Gatti L, Moore D T, Collichio F, Ollila D W, Sartor C I, Graham M L, Perou C M: The triple negative paradox: primary tumor chemosensitivity of breast cancer subtypes. Clin Cancer Res 2007, 13:2329-2334.
11. Rouzier R, Perou C M, Symmans W F, Ibrahim N, Cristofanilli M, Anderson K, Hess K R, Stec J, Ayers M, Wagner P, Morandi P, Fan C, Rabiul I, Ross J S, Hortobagyi G N, Pusztai L: Breast cancer molecular subtypes respond differently to preoperative chemotherapy. Clin Cancer Res 2005, 11:5678-5685.
12. von Minckwitz G, Untch M, Nüesch E, Loibl S, Kaufmann M, Kümmel S, Fasching P A, Eiermann W, Blohmer J U, Costa S D, Mehta K, Hilfrich J, Jackisch C, Gerber B, du Bois A, Huober J, Hanusch C, Konecny G, Fett W, Stickeler E, Harbeck N, Müller V, Jüni P: Impact of treatment characteristics on response of different breast cancer phenotypes: pooled analysis of the German neo-adjuvant chemotherapy trials. Breast Cancer Res Treat 2011, 125:145-156.
13. Kaufman P A, Broadwater G, Lezon-Geyda K, Dressler L G, Berry D, Friedman P, Winer E P, Hudis C, Ellis M J, Seidman A D, Harris L N: CALGB 150002: Correlation of HER2 and chromosome 17 (ch17) copy number with trastuzumab (T) efficacy in CALGB 9840, paclitaxel (P) with or without T in HER2+ and HER2− metastatic breast cancer (MBC) [abstract]. J Clin Oncol 2007, 25:s1009.
14. Paik S, Kim C, Jeong J, Geyer C E, Romond E H, Mejia-Mejia O, Mamounas E P, Wickerham D, Costantino J P, Wolmark N: Benefit from adjuvant trastuzumab may not be confined to patients with IHC 3+ and/or FISH-positive tumors: Central testing results from NSABP B-31 [abstract]. J Clin Oncol 2007, 25:s511.
15. Paik S, Kim C, Wolmark N: HER2 status and benefit from adjuvant trastuzumab in breast cancer. N Engl J Med 2008, 358:1409-1411.
16. Russnes H G, Vollan H K, Lingjaerde O C, Krasnitz A, Lundin P, Naume B, Sçrlie T, Borgen E, Rye I H, Langerçd A, Chin S F, Teschendorff A E, Stephens P J, M\dot{a}nér S, Schlichting E, Baumbusch L O, K\dot{a}resen R, Stratton M P, Wigler M, Caldas C, Zetterberg A, Hicks J, Bçrresen-Dale A L: Genomic architecture characterizes tumor progression paths and fate in breast cancer patients. Sci Transl Med 2010, 2:38ra47.
17. Perou C M, Sçrlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lçnning P E, Bçrresen-Dale A L, Brown P O, Botstein D: Molecular portraits of human breast tumours. Nature 2000, 406:747-752.
18. Gatza M L, Lucas J E, Barry W T, Kim J W, Wang Q, Crawford M D, Datto M B, Kelley M, Mathey-Prevot B, Potti A, Nevins J R: A pathway-based classification of human breast cancer. Proc Natl Acad Sci USA 2010, 107:6994-6999.
19. Kim C, Paik S: Gene-expression-based prognostic assays for breast cancer. Nat Rev Clin Oncol 2010, 7:340-347.
20. Sotiriou C, Pusztai L: Gene-expression signatures in breast cancer. N Engl J Med 2009, 360:790-800.
21. Shi L, Reid L H, Jones W D, Shippy R, Warrington J A, Baker S C, Collins P J, de Longueville F, Kawasaki E S, Lee K Y, Luo Y, Sun Y A, Willey J C, Setterquist R A, Fischer G M, Tong W, Dragan Y P, Dix D J, Frueh F W, Goodsaid F M, Herman D, Jensen R V, Johnson C D, Lobenhofer E K, Puri R K, Scherf U, Thierry-Mieg J, Wang C, Wilson M, Wolber P K, Zhang L, Amur S, Bao W, Barbacioru C C, Bergstrom Lucas A, Bertholet V, Boysen C, Bromley B, Brown D, Brunner A, Canales R, Cao X M, Cebula T A, Chen J J, Cheng J, Chu T M, Chudin E, Corson J, Corton J C, Croner L J, Davies C, Davison T S, Delenstarr G, Deng X, Dorris D, Eklund A C, Fan X, Fang H, Fulmer-Smentek S, Fuscoe J C, Gallagher K, Ge W, Guo L, Guo X, Hager J, Haje P K, Han J, Han T, Harbottle H C, Harris S C, Hatchwell E, Hauser C A, Hester S, Hong H, Hurban P, Jackson S A, Ji H, Knight C R, Kuo W P, LeClerc J E, Levy S, Li Q Z, Liu C, Liu Y, Lombardi M J, Ma Y, Magnuson S R, Maqsodi B, McDaniel T, Mei N, Myklebost O, Baitang N, Novoradovskaya N, Orr M S, Osborn T W, Papallo A, Patterson T A, Perkins R G, Peters E H, Peterson R, Philips K L, Pine S P, Pusztai L, Qian F, Ren H, Rosen M, Rosenzweig B A, Samaha R R, Schena M, Schroth G P, Shchegrova S, Smith D D, Staedtler F, Su Z, Sun H, Szallasi Z, Tezak Z, Thierry-Mieg D, Thompson K L, Tikhonova I, Turpaz Y, Vallanat B, Van C, Walker S J, Wang S J, Wang Y, Wolfinger R, Wong, Wu J, Xiao C, Xie Q, Xu J, Yang W, Zhang L, Zhong S, Zong Y, Slikker W Jr; for the MAQC Consortium: The MicroArray Quality Control (MAQC) project shows inter- and intraplatform reproducibility of gene expression measurements. Nat Biotechnol 2006, 24:1151-1161.
22. Gene Expression Omnibus (GEO) [http://www.ncbi.nlm.nih.gov/geo/]
23. Parker J S, Mullins M, Cheang M C, Leung S, Voduc D, Vickery T, Davies S, Fauron C, He X, Hu Z, Quackenbush J F, Stijleman I J, Palazzo J, Marron J S, Nobel A B, Mardis E, Nielsen T O, Ellis M J, Perou C M, Bernard P S: Supervised risk predictor of breast cancer based on intrinsic subtypes. J Clin Oncol 2009, 27:1160-1167.
24. van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R: A gene-expression signature as a predictor of survival in breast cancer. N Engl J Med 2002, 347:1999-2009.
25. Wang Y, Klijn J G, Zhang Y, Sieuwerts A M, Look M P, Yang F, Talantov D, Timmermans M, Meijer-van Gelder M E, Yu J, Jatkoe T, Berns E M, Atkins D, Foekens J A: Gene-expression profiles to predict distant metastasis of lymph-node-negative primary breast cancer. Lancet 2005, 365:671-679.
26. Sotiriou C, Wirapati P, Loi S, Harris A, Fox S, Smeds J, Nordgren H, Farmer P, Praz V, Haibe-Kains B, Desmedt C, Larsimont D, Cardoso F, Peterse H, Nuyten D, Buyse M, Van de Vijver M J, Bergh J, Piccart M, Delorenzi M: Gene expression profiling in breast cancer: understanding the molecular basis of histologic grade to improve prognosis. J Natl Cancer Inst 2006, 98:262-272.
27. Loi S, Haibe-Kains B, Desmedt C, Lallemand F, Tutt A M, Gillet C, Ellis P, Harris A, Bergh J, Foekens J A, Klijn J G, Larsimont D, Buyse M, Bontempi G, Delorenzi M, Piccart M J, Sotiriou C: Definition of clinically distinct molecular subtypes in estrogen receptor-positive breast carcinomas through genomic grade. J Clin Oncol 2007, 25:1239-1246.
28. Loi S, Haibe-Kains B, Desmedt C, Wirapati P, Lallemand F, Tutt A M, Gillet C, Ellis P, Ryder K, Reid J F, Daidone M G, Pierotti M A, Berns E M, Jansen M P, Foekens J A, Delorenzi M, Bontempi G, Piccart M J, Sotiriou C: Predicting prognosis using molecular profiling in estrogen receptor-positive breast cancer treated with tamoxifen. BMC Genomics 2008, 9:239.
29. Miller L D, Smeds J, George J, Vega V B, Vergara L, Ploner A, Pawitan Y, Hall P, Klaar S, Liu E T, Bergh J: An expression signature for p53 status in human breast cancer predicts mutation status, transcriptional effects, and patient survival. Proc Natl Acad Sci USA 2005, 102:13550-13555.
30. Ivshina A V, George J, Senko O, Mow B, Putti T C, Smeds J, Lindahl T, Pawitan Y, Hall P, Nordgren H, Wong J E, Liu E T, Bergh J, Kuznetsov V A, Miller L D: Genetic reclassification of histologic grade delineates new clinical subtypes of breast cancer. Cancer Res 2006, 66:10292-10301
31. Schmidt M, Böhm D, von Törne C, Steiner E, Puhl A, Pilch H, Lehr H A, Hengstler J G, Kölbl H, Gehrmann M: The humoral immune system has a key prognostic impact in node-negative breast cancer. Cancer Res 2008, 68:5405-5413.
32. Sabatier R, Finetti P, Cervera N, Lambaudie E, Esterni B, Mamessier E, Tallet A, Chabannon C, Extra J M, Jacquemier J, Viens P, Birnbaum D, Bertucci F: A gene expression signature identifies two prognostic subgroups of basal breast cancer. Breast Cancer Res Treat 2011, 126:407-420.
33. Loi S, Haibe-Kains B, Majjaj S, Lallemand F, Durbecq V, Larsimont D, Gonzalez-Angulo A M, Pusztai L, Symmans W F, Bardelli A, Ellis P, Tutt A N, Gillett C E, Hennessy B T, Mills G B, Phillips W A, Piccart M J, Speed T P, McArthur G A, Sotiriou C: PIK3CA mutations associated with gene signature of low mTORC1 signaling and better outcomes in estrogen receptor-positive breast cancer. Proc Natl Acad Sci USA 2010, 107:10208-10213.
34. Desmedt C, Piette F, Loi S, Wang Y, Lallemand F, Haibe-Kains B, Viale G, Delorenzi M, Zhang Y, d'Assignies M S, Bergh J, Lidereau R, Ellis P, Harris A L, Klijn J G, Foekens J A, Cardoso F, Piccart M J, Buyse M, Sotiriou C; TRANSBIG Consortium: Strong time dependence of the 76-gene prognostic signature for node-negative breast cancer patients in the TRANSBIG multicenter independent validation series. Clin Cancer Res 2007, 13:3207-3214.
35. Pawitan Y, Bjohle J, Amler L, Borg A L, Egyhazi S, Hall P, Han X, Holmberg L, Huang F, Klaar S, Liu E T, Miller L, Nordgren H, Ploner A, Sandelin K, Shaw P M, Smeds J, Skoog L, Wedrén S, Bergh J: Gene expression profiling spares early breast cancer patients from adjuvant therapy: derived and validated in two population-based cohorts. Breast Cancer Res 2005, 7:R953-R964.
36. Hoadley K A, Weigman V J, Fan C, Sawyer L R, He X, Troester M A, Sartor C I, Rieger-House T, Bernard P S, Carey L A, Perou C M: EGFR associated expression profiles vary with breast tumor subtype. BMC Genomics 2007, 8:258.
37. Juul N, Szallasi Z, Eklund A C, Li Q, Burrell R A, Gerlinger M, Valero V, Andreopoulou E, Esteva F J, Symmans W F, Desmedt C, Haibe-Kains B, Sotiriou C, Pusztai L, Swanton C: Assessment of an RNA interference screen-derived mitotic and ceramide pathway metagene as a predictor of response to neoadjuvant paclitaxel for primary triple-negative breast cancer: a retrospective analysis of five clinical trials. Lancet Oncol 2010, 11:358-365.
38. Oh D S, Troester M A, Usary J, Hu Z, He X, Fan C, Wu J, Carey L A, Perou C M: Estrogen-regulated genes predict survival in hormone receptor-positive breast cancers. J Clin Oncol 2006, 24:1656-1664.
39. Ben-Porath I, Thomson M W, Carey V J, Ge R, Bell G W, Regev A, Weinberg R A: An embryonic stem cell-like gene expression signature in poorly differentiated aggressive human tumors. Nat Genet 2008, 40:499-507.
40. Liu R, Wang X, Chen G Y, Dalerba P, Gurney A, Hoey T, Sherlock G, Lewicki J, Shedden K, Clarke M F: The prognostic role of a gene signature from tumorigenic breast-cancer cells. N Engl J Med 2007, 356:217-226.
41. Finak G, Bertos N, Pepin F, Sadekova S, Souleimanova M, Zhao H, Chen H, Omeroglu G, Meterissian S, Omeroglu A, Hallett M, Park M: Stromal gene expression predicts clinical outcome in breast cancer. Nat Med 2008, 14:518-527.
42. Shipitsin M, Campbell L L, Argani P, Weremowicz S, Bloushtain-Qimron N, Yao J, Nikolskaya T, Serebryiskaya T, Beroukhim R, Hu M, Halushka M K, Sukumar S, Parker L M, Anderson K S, Harris L N, Garber J E, Richardson A L, Schnitt S J, Nikolsky Y, Gelman R S, Polyak K: Molecular definition of breast tumor heterogeneity. Cancer Cell 2007, 11:259-273.
43. Chang H Y, Sneddon J B, Alizadeh A A, Sood R, West R B, Montgomery K, Chi J T, van de Rijn M, Botstein D, Brown P O: Gene expression signature of fibroblast serum response predicts human cancer progression: similarities between tumors and wounds. PLoS Biol 2004, 2:E7.
44. Loberg R D, Bradley D A, Tomlins S A, Chinnaiyan A M, Pienta K J: The lethal phenotype of cancer: the molecular basis of death due to malignancy. CA Cancer J Clin 2007, 57:225-241.
45. Paik S, Shak S, Tang G, Kim C, Baker J, Cronin M, Baehner F L, Walker M G, Watson D, Park T, Hiller W, Fisher E R, Wickerham D L, Bryant J, Wolmark N: A multigene assay to predict recurrence of tamoxifen-treated, node-negative breast cancer. N Engl J Med 2004, 351:2817-2826.
46. Bild A H, Yao G, Chang J T, Wang Q, Potti A, Chasse D, Joshi M B, Harpole D, Lancaster J M, Berchuck A, Olson JA Jr, Marks J R, Dressman H K, West M, Nevins J R: Oncogenic pathway signatures in human cancers as a guide to targeted therapies. Nature 2006, 439:353-357.
47. Hayes D F: Contribution of biomarkers to personalized medicine. Breast Cancer Res 2010, 12 Suppl 4:S3.
48. Albain K S, Barlow W E, Shak S, Hortobagyi G N, Livingston R B, Yeh I T, Ravdin P, Bugarini R, Baehner F L, Davidson N E, Sledge G W, Winer E P, Hudis C, Ingle J N, Perez E A, Pritchard K I, Shepherd L, Gralow J R, Yoshizawa C, Allred D C, Osborne C K, Hayes D F: Prognostic and predictive value of the 21-gene recurrence score assay in postmenopausal women with node-positive, oestrogen-receptor-positive breast cancer on chemotherapy: a retrospective analysis of a randomised trial. Lancet Oncol 2010, 11:55-65.
49. Bonnefoi H, Underhill C, Iggo R, Cameron D: Predictive signatures for chemotherapy sensitivity in breast cancer: are they ready for use in the clinic? Eur J Cancer 2009, 45:1733-1743.
50. Dobbe E, Gurney K, Kiekow S, Lafferty J S, Kolesar J M: Gene-expression assays: new tools to individualize treatment of early-stage breast cancer. Am J Health Syst Pharm 2008, 65:23-28.
51. Chang H Y, Nuyten D S, Sneddon J B, Hastie T, Tibshirani R, Sçrlie T, Dai H, He Y D, van't Veer L J, Bartelink H, van de Rijn M, Brown P O, van de Vijver M J: Robustness, scalability, and integration of a wound-response gene expression signature in predicting breast cancer survival. Proc Natl Acad Sci USA 2005, 102:3738-3743.
52. MAQC Consortium: The MicroArray Quality Control (MAQC) project shows inter- and intraplatform reproducibility of gene expression measurements. Nat Biotechnol 2006, 24:1151-1161.
53. Sorlie T, Tibshirani R, Parker J, Hastie T, Marron J S, Nobel A, Deng S, Johnsen H, Pesich R, Geisler S, Demeter J, Perou C M, Lçnning P E, Brown P O, Bçrresen-Dale A L, Botstein D: Repeated observation of breast tumor subtypes in independent gene expression data sets. Proc Natl Acad Sci USA 2003, 100:8418-8423.
54. Bierie B, Chung C H, Parker J S, Stover D G, Cheng N, Chytil A, Aakre M, Shyr Y, Moses H L: Abrogation of TGF-beta signaling enhances chemokine production and correlates with prognosis in human breast cancer. J Clin Invest 2009, 119:1571-1582.
55. Gatza M L, Lucas J E, Barry W T, Kim J W, Wang Q, Crawford M D, Datto M B, Kelley M, Mathey-Prevot B, Potti A, Nevins J R: A pathway-based classification of human breast cancer. Proc Natl Acad Sci USA 2010, 107:6994-6999.
56. van't Veer, He Y D, van de Vijver M J, et al: Gene expression profiling predicts clinical outcome of breast cancer. Nature 2001, 415:530-536

1. A method for classifying a subject afflicted with breast cancer according to a ClinicoMolecular Triad Classification (CMTC)-1, CMTC-2 or CMTC-3 class, comprising:
(i) determining a subject expression profile, said subject expression profile comprising the mRNA expression levels of a plurality of genes that classify breast cancer into three groups by hierarchal clustering TN and Her2+ breast cancers into one class (CMTC genes), in a breast cancer cell sample taken from said subject;
(ii) calculating a measure of similarity between said subject expression profile, and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients; and
(iii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

2. The method of claim 1, wherein the plurality of genes comprises genes selected from Table 9.

3. The method of claim 1, the method comprising:
(i) determining a subject expression profile said subject expression profile comprising the mRNA expression levels of a plurality of genes, the plurality comprising at least 200, at least 300, at least 400, at least 500, at least 600, at least 700, or at least 800 genes, optionally at least 200, at least 300, at least 400, at least 500, at least 600, at least 700, or at least 800 or 803 of the genes listed in Table 9 in a breast cancer cell sample taken from said subject;
(ii) calculating a measure of similarity between said subject expression profile, and one or more of: a) a CMTC-1 reference profile, said CMTC-1 reference profile comprising expression levels of said plurality of genes that are average mRNA expression levels of said respective genes in breast cancer cells of a plurality of breast cancer patients having ER+ low proliferating breast cancer; b) a CMTC-2 reference profile, said CMTC-2 reference profile comprising expression levels of said plurality of genes that are average mRNA expression levels of the respective genes in breast cancer cells of a plurality of breast cancer patients having ER+ high proliferating breast cancer; and c) a CMTC-3 reference profile, said CMTC-3 reference profile comprising expression levels of said plurality of genes that are average mRNA expression levels of said respective genes in breast cancer cells of a plurality of triple negative and HER2+ breast cancer patients; and
(iii) classifying said subject as falling in said CMTC-1 class if said subject expression profile is most similar to said CMTC-1 reference profile, classifying said subject as falling in said CMTC-2 class if said subject expression profile is most similar to said CMTC-2 reference profile or classifying said subject as falling in said CMTC-3 class if said subject expression profile is most similar to said CMTC-3 reference profile.

4. The method of claim 1, wherein said similarity is assessed by calculating a correlation coefficient between the subject expression profiles and the one or more of CMTC-1, CMTC-2 and CMTC-3 reference profiles, wherein the subject is classified as falling in the class that has the highest correlation coefficient with the subject expression profile.

5. The method of claim 1, wherein step (iii) additionally or alternatively comprises classifying said subject as having a poor prognosis if said subject expression profile has a high similarity and/or is most similar to said CMTC-3 reference profile or said CMTC-2 reference profile, or classifying said subject as having a good prognosis if said subject expression profile as a high similarity and/or is most similar to said CMTC-1 reference profile; and providing said prognosis classification to the subject.

6. The method of claim 1, wherein said plurality of genes comprises at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes and optionally at least 97%, 98%, 99% or 100% of the genes listed in Table 9.

7. The method of claim 1, further comprising (iii) displaying; or outputting to a user interface device, a computer-readable storage medium, or a local or remote computer system, the classification produced by said classifying step (ii).

8. The method of claim 1, the method comprising:
a. obtaining a breast cancer cell sample from the subject;
b. assaying the sample and determining a subject expression profile, said subject expression profile comprising the mRNA expression levels of a plurality of genes, the plurality comprising optionally at least 200, at least 300, at least 400, at least 500, at least 600, at least 700, or at least 800, genes, optionally at least 200, at least 300, at least 400, at least 500, at least 600, at least 700, at least 800, or 803 of the genes listed in Table 9 in a breast cancer cell sample taken from said subject
c. comparing the subject expression profile to one or more of a CMTC-1, CMTC-2 and/or CMTC-3 reference profile, said CMTC-1 reference profile comprising expression levels of said plurality of genes that are average mRNA expression levels of said respective genes in breast cancer cells of a plurality of ER+ low proliferating breast cancer patients, said CMTC-2 reference profile comprising expression levels of said plurality of genes that are average mRNA expression levels of said respective genes in breast cancer cells of a plurality of breast cancer patients having ER+ high proliferating breast cancer; and said CMTC-3 reference profile comprising expression levels of said plurality of genes that are average mRNA expression levels of said respective genes in breast cancer cells of a plurality of triple negative and HER2+ breast cancer patients;
d. classifying said subject as falling within a CMTC-1 class if said subject expression profile has a higher similarity to the CMTC-1 reference profile than the CMTC-2 or CMTC-3 reference profiles; classifying said subject as falling within a CMTC-2 class if said subject profile has a higher similarity to the CMTC-2 reference profile than the CMTC-1 or CMTC-3 reference profiles; and classifying said subject as falling within a CMTC-3 class if said subject profile has a higher similarity to the CMTC-3 reference profile than the CMTC-1 or CMTC-2 reference profiles.

9. The method of claim 1, wherein said CMTC reference profile comprises for at least 200, at least 300, at least 400, at least 500, at least 600, at least 700, at least 800, or all 803 genes in Table 9 or for at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the genes in Table 9, respective centroid values listed in Table 9.

10. The method of claim 1, wherein said expression level of each gene in said subject expression profile is a relative expression level of said gene in said breast cancer cell sample versus expression level of said gene in a reference pool, optionally represented as a log ratio and/or, wherein said reference profile comprising expression levels of the plurality of genes is an error-weighted average.

11. The method of claim 1, further comprising the step of determining oncogenic or cellular pathway activation.

12. The method of claim 1, wherein the method is used to select a suitable treatment.

13. A method for monitoring a response to a cancer treatment in a subject afflicted with breast cancer, comprising:
a. collecting a first breast cancer cell sample from the subject before the subject has received the cancer treatment or during treatment and collecting a subsequent breast cancer cell sample from the subject after the subject has received at least one cancer treatment dose;
b. assaying said first sample and determining a first subject expression profile, said first subject expression profile comprising the mRNA expression levels of a plurality of genes of said first breast cancer cell sample and assaying and determining a second subject expression profile, said second subject expression profile comprising the mRNA expression levels of said plurality of genes of said subsequent breast cancer cell sample, said plurality of genes comprising at least 200 genes listed in Table 9;
c. classifying said subject as having a good prognosis, intermediate-poor prognosis or a poor prognosis or CMTC class based on said first subject expression profile and classifying said subject as having a good prognosis, intermediate-poor prognosis or a poor prognosis or CMTC class based on said second subject expression profile according to the method of claim 1;
d. and/or calculating a first sample subject expression profile score and a subsequent sample subject expression profile score;
wherein a lower subsequent sample expression profile score or better prognosis class compared to the first sample expression profile score is indicative of a positive response, and a higher subsequent sample expression profile score or worse class compared to said first sample subject expression profile score is indicative of a negative response.

14. The method of claim 1, wherein each of said mRNA expression levels is determined using one or more probes and/or one or more probe sets, optionally wherein the one or more polynucleotide probes and/or the one or more polynucleotide probe sets are selected from the probes identified by number in Table 9.

15. The method of claim 1, wherein the mRNA expression level is determined using an array and/or PCR method, optionally multiplex PCR, optionally, wherein the array is selected from an Illumina™ Human Ref-8 expression microarray, an Agilent™ Hu25K microarray and an Affymetrix™ U133 or other genome wide microarray optionally comprising probes for detecting gene expression of at least 25%, at least 30%, at least 35%, at least 40%, at least 45%, at least 50% of the genes in Table 9.

16. The method of claim 5 comprising: (a) contacting first nucleic acids derived from mRNA of a breast cancer cell sample taken from said subject, and optionally a second nucleic acids derived from mRNA of two or more breast cancer cell samples from breast cancer patients who have recurrence within a predetermined period from initial diagnosis of breast cancer and/or known ER/PR/HER2 clinical status, with an array under conditions such that hybridization can occur, wherein the first nucleic acids are labeled with a first fluorescent label, and the optional second nucleic acids are labeled with e second fluorescent label, detecting at each of a plurality of discrete loci on said array a first fluorescent emission signal from said first nucleic acids and optionally a second fluorescent emission signal from said second nucleic acids that are bound to said array under said conditions, wherein said array comprises at least 200 of the genes listed in Table 9; (b) calculating a first measure of similarity between said first fluorescent emission signals and said second fluorescent emission signals across said at least 200 genes or calculating one or more measures of similarity between said first fluorescent emission signals and one or more reference profiles; (c) classifying said subject based on the similarity between said first fluorescent emission signals and said second fluorescent emission signals across said at least 200 genes or based on the similarity between said first fluorescent emission signals and said one or more reference profiles across said at least 200 genes (e.g. CMTC-1, CMTC-2, CMTC-3 reference profiles) wherein said individual is classified as having a good prognosis if said subject expression profile is most similar to a good prognosis reference profile an intermediate-poor prognosis if said subject expression profile is most similar to said intermediate-poor prognosis reference profile or a poor prognosis if said subject expression profile is most similar to said poor prognosis reference profile; and (d) displaying; or outputting to a user interface device, a computer readable storage medium, or a local or remote computer system; the classification produced by said classifying step (c).

17. A method of treating a subject afflicted with breast cancer, comprising classifying said subject according to the method of claim 1, and providing a suitable cancer treatment to the subject in need thereof according to the class determined.

18. A method for classifying a remotely obtained breast cancer sample according to CMTC and providing access to the CMTC classification of the breast cancer cell sample, the method comprising:
a) receiving a remotely obtained breast cancer cell sample and a breast cancer cell sample identifier associated to the breast cancer cell sample;
b) determining on-site the expression levels for a plurality of genes of the received cell sample;
c) classifying the breast cancer cell sample according to claim 1;
d) providing access to the CMTC classification for the breast cancer cell sample.

19. A kit for determining CMTC class in a subject afflicted with breast cancer according to the method of claim 18 comprising one or more of:
a) a needle or other breast cancer cell sample obtainer;
b) tissue RNA preservative solution;
c) breast cancer cell sample identifier;
d) vial such as a cryovial; and
e) instructions.

20. The method of claim 13, wherein each of said mRNA expression levels is determined using one or more probes and/or one or more probe sets, optionally wherein the one or more polynucleotide probes and/or the one or more polynucleotide probe sets are selected from the probes identified by number in Table 9; or wherein the mRNA expression level is determined using an array and/or PCR method, optionally multiplex PCR, optionally, wherein the array is selected from an Illumina™ Human Ref-8 expression microarray, an Agilent™ Hu25K microarray and an Affymetrix™ U133 or other genome wide microarray optionally comprising probes for detecting gene expression of at least 25%, at least 30%, at least 35%, at least 40%, at least 45%, at least 50% of the genes in Table 9.
